(19) United States
(12) Patent Application Publication
(10) Pub. No.: US 2018/0256644 A1
(43) Pub. Date: Sep. 13, 2018

(54) TUNABLE VARIANT IMMUNOGLOBULIN SUPERFAMILY DOMAINS AND ENGINEERED CELL THERAPY

(71) Applicant: ALPINE IMMUNE SCIENCES, INC., Seattle, WA (US)

(72) Inventors: Ryan SWANSON, Seattle, WA (US); Michael KORNACKER, Seattle, WA (US)

(73) Assignee: ALPINE IMMUNE SCIENCES, INC., Seattle, WA (US)

(21) Appl. No.: 15/759,812

(22) PCT Filed: Sep. 14, 2016
(86) PCT No.: PCT/US2016/051786

§ 371 Date: Mar. 13, 2018

Related U.S. Application Data
(60) Provisional application No. 62/218,531, filed on Sep. 14, 2015.
(60) Provisional application No. 62/323,608, filed on Apr. 15, 2016.
(60) Provisional application No. 62/323,595, filed on Apr. 15, 2016.
(60) Provisional application No. 62/367,822, filed on Jul. 28, 2016.
(60) Provisional application No. 62/367,819, filed on Jul. 28, 2016.

(51) Int. Cl. A61K 35/17; A61K 39/00; C12N 5/0783; C12N 5/0781; C07K 19/00; C07K 14/705; A61K 38/17

(52) U.S. Cl. CPC A61K 35/17; A61K 39/0011; C12N 5/0638; A61P 37/00; C07K 19/00; C07K 14/70517; A61K 38/1774; C12N 5/0635

(57) ABSTRACT

Provided are transmembrane immunomodulatory proteins, nucleic acids encoding such proteins and cells engineered with the proteins. The transmembrane immunomodulatory proteins and engineered cells provide therapeutic utility for a variety of immunological and oncological conditions. Compositions and methods for making and using such proteins are provided.

### CROSS-REFERENCE TO RELATED APPLICATIONS

This application claims priority from U.S. provisional application No. 62/218,531 filed Sep. 14, 2015, entitled “Tunable Variant Immunoglobulin Superfamily Domains and Engineered Cell Therapy,” U.S. provisional application No. 62/323,608 filed Apr. 15, 2016, entitled “ICOS Ligand Variant Immunomodulatory Proteins and Uses Thereof,” to U.S. provisional application No. 62/323,595 filed Apr. 15, 2016, entitled “CD80 Variant Immunomodulatory Proteins and Uses Thereof,” U.S. provisional application No. 62/367,822 filed Jul. 28, 2016, entitled “CD155 Variant Immunomodulatory Proteins and Uses Thereof,” and U.S. provisional application No. 62/367,819 filed Jul. 28, 2016, entitled “CD112 Variant Immunomodulatory Proteins and Uses Thereof,” the contents of each of which are incorporated by reference in their entirety.

### INCORPORATION BY REFERENCE OF SEQUENCE LISTING

The present application is being filed along with a Sequence Listing in electronic format. The Sequence Listing is provided as a file entitled 761612000240SeqList.TXT, created Sep. 10, 2016 which is 750,987 bytes in size. The information in the electronic format of the Sequence Listing is incorporated by reference in its entirety.

### FIELD

The present invention relates to transmembrane immunomodulatory proteins (TIPs) and immune cells engineered to express such immunomodulatory proteins for modulating immune response in the treatment of cancer and immunological diseases.

### BACKGROUND

Modulation of the immune response by intervening in the processes that occur in the immunological synapse (IS) formed by and between antigen-presenting cells (APCs) or target cells and lymphocytes is of increasing medical interest. Currently, biologics used to enhance or suppress immune responses have generally been limited to immunoglobulins (e.g., anti-PD-1 mAbs) or soluble receptors (e.g., Fc-CTLA4). Soluble receptors suffer from a number of deficiencies. While useful for antagonizing interactions between proteins, soluble receptors often lack the ability to agonize such interactions. Antibodies have proven less limited in this regard and examples of both agonistic and antagonistic antibodies are known in the art. Nevertheless, both soluble receptors and antibodies lack important attributes that are critical to function in the IS. Mechanistically, cell surface proteins in the IS can involve the coordinated and often simultaneous interaction of multiple protein targets with a single protein to which they bind. IS interactions occur in close association with the junction of two cells, and a single protein in this structure can interact with both a protein on the same cell (cis) as well as a protein on the associated cell (trans), likely at the same time. Thus, there is a need for improved molecules for modulation of immune responses. Provided are embodiments that meet such needs.

### SUMMARY

Provided herein are transmembrane immunomodulatory proteins (TIP) containing (1) an ectodomain comprising at least one non-immunoglobulin affinity-modified immunoglobulin superfamily (IgSF) domain containing one or more amino acid substitution(s) in a wild-type IgSF domain, wherein the at least one affinity-modified IgSF domain specifically binds at least one cell surface cognate binding partner of the wild-type IgSF domain and (2) a transmembrane domain.

In some of any such embodiments, the at least one cell surface cognate binding partner is expressed on a mammalian cell. In some of any such embodiments, the mammalian cell is an antigen presenting cell (APC), a tumor cell, or a lymphocyte. In some embodiments, the lymphocyte is a T-cell. In some of any such embodiments, the mammalian cell is a mouse, rat, cynomolgus monkey, or human cell.

In some of any such embodiments, the at least one affinity modified IgSF domain has increased binding affinity to the at least one cell surface cognate binding partner compared with the reference wild-type IgSF domain. In some of any such embodiments, specific binding of the transmembrane immunomodulatory protein containing the at least one affinity-modified IgSF domain modulates immunological activity of the mammalian cell compared with the reference transmembrane domain containing the wild-type IgSF domain. In some of any such embodiments, specific binding of the transmembrane immunomodulatory protein containing the at least one affinity-modified IgSF domain increases immunological activity of the mammalian cell compared with the reference transmembrane domain containing the wild-type IgSF domain. In some of any such embodiments, specific binding of the transmembrane immunomodulatory protein attenuates immunological activity of the mammalian cell compared with the reference transmembrane domain containing the wild-type IgSF domain.

In some of any such embodiments, the wild-type IgSF domain is from an IgSF family member of a family selected from Signal-Regulatory Protein (SIRP) Family, Triggering Receptor Expressed On Myeloid Cells Like (TREML) Family, Carcinoembryonic Antigen-related Cell Adhesion Molecule (CEACAM) Family, Sialic Acid Binding Ig-Like Lectin (SIGLEC) Family, Butyrophilin Family, B7 family, CD28 family, V-set and Immunoglobulin Domain Containing (VSIG) family, V-set transmembrane Domain (VSTM) family, Major Histocompatibility Complex (MHC) family, Signaling lymphocytic activation molecule (SLAM) family, Leukocyte immunoglobulin-like receptor (LIR), Nectin (Nec) family, Nectin-like (NECL) family, Poliovirus receptor related (PVR) family, Natural cytotoxicity triggering receptor (NCR) family, T cell immunoglobulin and mucin (TIM) family or Killer-cell immunoglobulin-like receptors (KIR) family. In some of any such embodiments, the wild-type IgSF domain is from an IgSF member selected from CD80, CD86, PD-L1, PD-L2, ICOS Ligand, B7-H3, B7-H4, CD28, CTLA4, PD-1, ICOS, BTLA, CD4, CD8-alpha, CD8-beta, LAG3, TIM-3, CEACAM1, TIGIT, PVR, PVRL2, CD226, CD2, CD160, CD200, CD200R or Nkp30. In some of any such embodiments, the wild-type IgSF domain is a human IgSF member.

In some of any such embodiments, the at least one affinity modified IgSF domain has at least 90% sequence identity to a wild-type IgSF domain or a specific binding fragment thereof contained in the sequence of amino acids set forth in any of SEQ ID NOS: 1-54. In some of any such embodiments, the transmembrane immunomodulatory protein has at least 90% sequence identity to the amino acid sequence selected from any of SEQ ID NOS: 393-419.

In some of any such embodiments, the at least one cell surface cognate binding partner is a stimulatory receptor expressed on a T-cell and the at least one affinity-modified IgSF domain has increased binding affinity to the stimulatory receptor compared to the affinity of the wild-type IgSF domain. In some embodiments, binding of the affinity-modified IgSF domain to the stimulatory receptor increases immunological activity of the T-cell.

In some of any such embodiments, provided is a transmembrane immunomodulatory protein (TIP) comprising: an ectodomain, wherein the ectodomain comprises at least one non-immunoglobulin affinity-modified immunoglobulin superfamily (IgSF) domain; and a transmembrane domain, wherein: the TIP is expressed on a first T-cell; the affinity-modified IgSF domain specifically binds at least one counter-structure expressed on a mammalian cell; the mammalian cell is an antigen presenting cell (APC), a tumor cell, or a second T-cell; and specific binding of the affinity-modified IgSF domain to a counter-structure modulates immunological activity of the mammalian cell. In some embodiments, the TIP comprises a first affinity-modified IgSF domain, wherein the counter-structure expressed on the mammalian cell is a stimulatory counter-structure expressed on the second T-cell; and the first affinity-modified IgSF domain specifically binds to the stimulatory counter-structure and increases immunomodulatory activity of the second T-cell.

In some cases, the stimulatory receptor is CD28, ICOS or CD226. In some of any such embodiments, the at least one affinity-modified IgSF domain is an affinity modified B7-1 IgSF domain and the stimulatory receptor is CD28. In some of any such embodiments, the at least one affinity-modified IgSF domain is an affinity modified ICOSL IgSF domain and the stimulatory receptor is ICOS. In some of any such embodiments, the affinity-modified IgSF domain is an affinity modified ICOSL IgSF domain and the stimulatory receptor is CD28. In some of any such embodiments, the at least one affinity-modified IgSF domain is an affinity-modified ICOSL IgSF domain that has increased binding affinity to at least one of: ICOS and CD28. In some of any such embodiments, the affinity modified IgSF domain is an affinity modified ICOSL IgV IgSF domain with increased binding affinity to both ICOS and CD28. In some of any such embodiments, the affinity-modified IgSF domain does not substantially specifically bind to CTLA-4 or exhibits decreased binding affinity to CTLA-4 compared to the wild-type IgSF domain.

In some of any such embodiments, the at least one affinity-modified IgSF domain specifically binds to no more than one cell surface cognate binding partner. In some of any such embodiments, the transmembrane immunomodulatory protein specifically binds to no more than one cell surface cognate binding partner. In some of any such embodiments, the at least one affinity-modified domain specifically binds to at least two cell surface cognate binding partners.

In some of any such embodiments, the first cell surface cognate binding partner is a stimulatory receptor expressed on a T cell; and the second cell surface cognate binding partner is an inhibitory ligand of an inhibitory receptor, wherein the inhibitory receptor is expressed on a T-cell.

In some of any such embodiments, binding of the affinity-modified domain to the inhibitory ligand competitively inhibits binding of the inhibitory ligand to the inhibitory receptor. In some embodiments, the inhibitory receptor is PD-1, CTLA-4, LAG-3, TIGIT, CD96, CD112R, BTLA, CD160 or TIM-3; or the ligand of the inhibitory receptor is PD-L1, PD-L2, B7-1, B7-2, HVEM, MHC class II, PVR, CEACAM-1 or GAL9. In some of any such embodiments, the affinity modified IgSF domain is an affinity modified B7-1 domain and the stimulatory receptor is CD28. In some of any such embodiments, the inhibitory ligand is PD-L1 and the inhibitory receptor is PD-1.

In some of any such embodiments, the affinity-modified IgSF domain exhibits decreased binding affinity to CTLA-4 compared to the wild-type IgSF domain for CTLA-4. In some of any such embodiments, the affinity-modified IgSF domain does not substantially specifically bind to CTLA-4. In some of any such embodiments, the affinity modified IgSF domain is an affinity modified CD155 IgSF domain or an affinity modified CD112 IgSF domain and the stimulatory receptor is CD226. In some of any such embodiments, the affinity-modified IgSF domain exhibits decreased binding affinity to TIGIT (T-cell immunoreceptor with Ig and ITIM domains) compared to the affinity of the wild-type IgSF domain.

In some of any such embodiments, the at least one affinity-modified IgSF domain specifically binds to a cell surface cognate binding partner that is a tumor specific antigen. In some embodiments, the tumor specific antigen is B7-H6.

In some of any such embodiments, the affinity-modified IgSF domain is an affinity modified Nkp30 IgSF domain. In some of any such embodiments, the at least one affinity-modified IgSF domain is a first affinity-modified IgSF domain and the ectodomain contains a second affinity-modified IgSF domain. In some embodiments, the first and second affinity-modified IgSF domain are different. In some of any such embodiments, the first affinity-modified IgSF domain and the second affinity-modified IgSF domain each contain one or more amino acid different substitutions in the same wild-type IgSF domain. In some of any such embodiments, the first affinity-modified IgSF domain and the second affinity-modified IgSF domain each contain one or more amino acid substitutions in a different wild-type IgSF domain.

In some of any such embodiments, the transmembrane immunomodulatory protein further contains an endodomain or cytoplasmic signaling domains. In some embodiments, the endodomain is the endodomain from the wild-type IgSF member containing the wild-type IgSF domain or is a functionally active portion thereof. In some embodiments, the transmembrane immunomodulatory protein is a chimeric receptor, wherein the endodomain is not the endodomain from the wild-type IgSF member containing the wild-type IgSF domain. In some of any such embodiments, the endodomain contains at least one ITAM (immunoreceptor tyrosine-based activation motif)-containing signaling domain. In some of any such embodiments, the endodomain contains a CD3-zeta signaling domain. In some of any such embodiments, the endodomain further contains at least one of: a CD28 costimulatory domain, an ICOS signaling domain, an OX40 signaling domain, and a 41BB signaling domain.

In some of any such embodiments, the wild-type IgSF domain is from an IgSF member that is an inhibitory receptor containing an ITIM signaling domain. In some embodiments, the inhibitory receptor is PD-1, CTLA-4, LAG3, TIGIT, TIM-3, or BTLA and the at least one affinity-modified IgSF domain is an affinity-modified IgSF domain of PD-1, CTLA-4, LAG3, TIGIT, TIM-3, or BTLA, respectively. In some of any such embodiments, the inhibitory receptor is PD-1 and the at least one affinity-modified IgSF domain is an affinity-modified IgSF of PD-1. In some of any such embodiments, the affinity-modified IgSF domain has increased binding affinity for a trans surface cognate binding partner compared to the wildtype IgSF domain, whereby the increased binding affinity competitively inhibits binding of the trans surface cognate binding partner to the inhibitory receptor.

In some of any such embodiments, the transmembrane immunomodulatory protein does not contain an endodomain, ITIM or cytoplasmic signaling domains.

In some of any such embodiments, the affinity modified IgSF domain differs by no more than ten amino acid substitutions from the wildtype IgSF domain. In some of any such embodiments, the affinity modified IgSF domain differs by no more than five amino acid substitutions from the wildtype IgSF domain.

In some of any such embodiments, the affinity-modified IgSF domain is or contains an affinity modified IgV domain, affinity modified IgC1 domain or an affinity modified IgC2 domain or is a specific binding fragment thereof containing the one or more amino acid substitutions. In some of any such embodiments, the ectodomain further contains one or more non-affinity modified IgSF domains.

In some of any such embodiments, the one or more non-affinity modified IgSF domains is from a wild-type IgSF member containing the wild-type IgSF domain. In some of any such embodiments, the transmembrane domain is the native transmembrane domain from the corresponding wild-type IgSF member. In some of any such embodiments, the transmembrane domain is not the native transmembrane domain from the corresponding wild-type IgSF member. In some embodiments, the transmembrane protein is a transmembrane protein derived from CD8.

In another aspect, the present invention relates to a recombinant nucleic acid encoding any of the transmembrane immunomodulatory proteins summarized above.

In another aspect, the present invention relates to a recombinant expression vector containing any of the nucleic acids summarized above.

In another aspect, the present invention relates to a recombinant expression vector containing a nucleic acid encoding any of the transmembrane immunomodulatory proteins summarized above.

In another aspect, the present invention relates to a recombinant host cell containing any of the expression vectors summarized above.

In another aspect, the present invention relates to a recombinant host cell containing a nucleic acid described above. In some of any such embodiments, the host cell is a mammalian host cell. In some of any such embodiments, the mammalian host cell is a human host cell.

In another aspect, the present invention relates to an engineered cell containing any of the transmembrane immunomodulatory proteins described above. In some of any such embodiments, the cell is an immune cell. In some of any such embodiments, the cell is a lymphocyte. In some embodiments, the lymphocyte is a T cell, a B cell or an NK cell. In some of any such embodiments, the cell is a T cell. In some of any such embodiments, the T cells is CD4+ or CD8+. In some of any such embodiments, the cell is an antigen presenting cell.

In some of any such embodiments, the engineered cell further contains a chimeric antigen receptor (CAR) or an engineered T-cell receptor (TCR).

In another aspect, the present invention relates to a pharmaceutical composition containing any of the cells described above and a pharmaceutically acceptable carrier. In some embodiments, the pharmaceutical composition is sterile.

In some embodiments, provided herein is a method of modulating an immune response in a mammalian subject, involving administering a cell according to any one of the embodiments described above or a pharmaceutical composition according to any one of the embodiments described above to the subject. In some of any such embodiments, modulating the immune response treats a disease or disorder in the subject. In some of any such embodiments, the modulated immune response is increased. In some embodiments, the disease or disorder is a tumor. In some of any such embodiments, the disease or disorder is a cancer. In some of any such embodiments, the disease or disorder is melanoma, lung cancer, bladder cancer, or a hematological malignancy.

In some of any such embodiments, the modulated immune response is decreased. In some of any such embodiments, the disease or disorder is an inflammatory disease or condition. In some of any such embodiments, the disease or condition is Crohn's disease, ulcerative colitis, multiple sclerosis, asthma, rheumatoid arthritis, or psoriasis.

In some of any such embodiments, the subject is human. In some of any such embodiments, the cell is autologous to the subject. In some of any such embodiments, the cell is allogenic to the subject.

### BRIEF DESCRIPTION OF THE DRAWINGS

FIG. 1A depicts results of a competition binding assay for binding of biotinylated recombinant CD28 Fc fusion protein (rCD28.Fc) to immobilized CD80 variant A91G ECD-Fc fusion molecule in the presence of unlabeled recombinant human PD-L1-his, human CTLA-4-his or human-PD-L2-Fc fusion protein.

FIG. 1B depicts results of a competition binding assay for binding of biotinylated recombinant human PD-L1-his monomeric protein to immobilized CD80 variant A91G ECD-Fc fusion molecule in the presence of unlabeled recombinant human rCD28.Fc, human CTLA-4.Fc or human PD-L2.Fc

FIG. 2 depicts impedance results reflecting cytotoxic killing activity of cells engineered with an anti-CD19 chimeric antigen receptor (CAR) alone or with an exemplary transmembrane immunomodulatory TIP (CD80-TIP or ICOSL-TIP) or the corresponding CD80 or ICOSL wild-type transmembrane protein following co-culture with target antigen-expressing cells. Impedance was assessed using the Acea Real-Time Cell Analyzer (RTCA), which measures the impedance variations in the culture media of a 96-well microelectronic plate (E-plate).

### DETAILED DESCRIPTION

Provided herein are transmembrane immunomodulatory proteins (TIPs) and cells, such as immune cells, engineered to express such TIPs. In some embodiments, the TIP contains an extracellular ligand binding domain that contains an affinity-modified IgSF domain and that is capable of binding to one or more protein ligands, and generally two or more protein ligands. In some embodiments, the protein ligands are cell surface proteins expressed by immune cells that engage with one or more other immune receptor, e.g. on lymphocytes, to induce inhibitory or activating signals. For example, the interaction of certain receptors on lymphocytes with their cognate cell surface ligands to form an immunological synapse (IS) between antigen-presenting cells (APCs) or target cells and lymphocytes can provide costimulatory or inhibitory signals that can regulate the immune system. In some aspects, TIP-engineered cells expressing a TIP provided herein can alter the interaction of cell surface protein ligands with their receptors to thereby modulate immune cells, such as T cell, activity. In some embodiments, the binding of the TIP to a ligand or ligands modulates, e.g. induces, enhances or suppresses, immunological immune responses of the immune cell in which it is expressed or a cell to which the TIP expressed on the cell specifically binds.

In some embodiments, under normal physiological conditions, the T cell-mediated immune response is initiated by antigen recognition by the T cell receptor (TCR) and is regulated by a balance of co-stimulatory and inhibitory signals (i.e., immune checkpoint proteins). The immune system relies on immune checkpoints to prevent autoimmunity (i.e., self-tolerance) and to protect tissues from excessive damage during an immune response, for example during an attack against a pathogenic infection. In some cases, however, these immunomodulatory proteins can be dysregulated in diseases and conditions, including tumors, as a mechanism for evading the immune system.

Thus, in some aspects, immunotherapy that alters immune cell activity, such as T cell activity, can treat certain diseases and conditions in which the immune response is dysregulated. Therapeutic approaches that seek to modulate interactions in the IS would benefit from the ability to bind multiple IS targets simultaneously and in a manner that is sensitive to temporal sequence and spatial orientation. Current therapeutic approaches fall short of this goal. Instead, soluble receptors and antibodies typically bind no more than a single target protein at a time. This may be due to the absence of more than a single target species. Additionally, wild-type receptors and ligands possess low affinities for cognate binding partners, which preclude their use as soluble therapeutics.

Less trivially, however, soluble receptors and antibodies generally bind competitively (e.g., to no more than one target species at a time) and therefore lack the ability to simultaneously bind multiple targets. And while bispecific antibodies, as well as modalities comprising dual antigen binding regions, can bind to more than one target molecule simultaneously, the three-dimensional configuration typical of these modalities often precludes them intervening in key processes occurring in the IS in a manner consistent with their temporal and spatial requirements.

What is needed is an entirely new class of therapeutic molecules that have the specificity and affinity of antibodies or soluble receptors but, in addition, maintain the size, volume, and spatial orientation constraints required in the IS. Further, such therapeutics would have the ability to bind to their targets non-competitively as well as competitively. A molecule with these properties would therefore have novel function in the ability to integrate into multi-protein complexes at IS and generate the desired binding configuration and resulting biological activity.

To this end, emerging immuno-oncology therapeutic regimes need to safely break tumor-induced T cell tolerance. Current state-of-the-art immuno-therapeutics block PD-1 or CTLA4, central inhibitory molecules of the B7/CD28 family that are known to limit T cell effector function. While antagonistic antibodies against such single targets function to disrupt immune synapse checkpoint signaling complexes, they fall short of simultaneously activating T cells. Conversely, bispecific antibody approaches activate T cells, but fall short of simultaneously blocking inhibitory ligands that regulate the induced signal.

To address these shortcomings, provided are immunotherapies, such as cell therapies, that can modulate immune cell activities. In some embodiments, the provided immunotherapies can enhance immune cells signaling, such as T-cell activation signaling, and/or can block inhibitory regulation, which, in some cases, can occur simultaneously. In some embodiments, the provided immunotherapies relate to immunoglobulin superfamily (IgSF) components of the immune synapse that are known to have a dual role in both T-cell activation and blocking of inhibitory ligands. In some aspects, IgSF based-cell therapies engineered from immune system ligands, such as human immune system ligands themselves are more likely to retain their ability to normally assemble into key pathways of the immune synapse and maintain normal interactions and regulatory functions in ways that antibodies or next-generation bi-specific reagents cannot. This is due to the relatively large size of antibodies as well as from the fact they are not natural components of the immune synapse. These unique features of human immune system ligands, and cells engineered to express affinity-modified variants of such ligands, promise to provide a new level of immunotherapeutic efficacy and safety. In particular aspects, the provided TIP-engineered cells provide an immunotherapy platform using affinity modified native immune ligands to generate immunotherapy biologics that bind with tunable affinities to one or more of their cognate immune receptors in the treatment of a variety of oncological and immunological indications.

All publications, including patents, patent applications scientific articles and databases, mentioned in this specification are herein incorporated by reference in their entirety for all purposes to the same extent as if each individual publication, including patent, patent application, scientific article or database, were specifically and individually indicated to be incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms used throughout this specification are defined as follows unless otherwise limited in specific instances. As used in the specification and the appended claims, the singular forms “a,” “an,” and “the” include plural referents unless the context clearly dictates otherwise. Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

The term “affinity modified” as used in the context of an immunoglobulin superfamily domain, means a mammalian immunoglobulin superfamily (IgSF) domain having an altered amino acid sequence (relative to the corresponding wild-type parental or unmodified IgSF domain) such that it has an increased or decreased binding affinity or avidity to at least one of its cognate binding partners (alternatively “counter-structures”) compared to the parental wild-type or unmodified (i.e., non-affinity modified) IgSF control domain. In some embodiments, the affinity-modified IgSF domain can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions, in a wildtype or unmodified IgSF domain. An increase or decrease in binding affinity or avidity can be determined using well known binding assays such as flow cytometry. Larsen et al., American Journal of Transplantation, Vol 5: 443-453 (2005). See also, Linsley et al., Immunity, 1: 7930801 (1994). An increase in a protein's binding affinity or avidity to its cognate binding partner(s) is to a value at least 10% greater than that of the wild-type IgSF domain control and in some embodiments, at least 20%, 30%, 40%, 50%, 100%, 200%, 300%, 500%, 1000%, 5000%, or 10000% greater than that of the wild-type IgSF domain control value. A decrease in a protein's binding affinity or avidity to at least one of its cognate binding partner is to a value no greater than 90% of the control but no less than 10% of the wild-type IgSF domain control value, and in some embodiments no greater than 80%, 70% 60%, 50%, 40%, 30%, or 20% but no less than 10% of the wild-type IgSF domain control value. An affinity-modified protein is altered in primary amino acid sequence by substitution, addition, or deletion of amino acid residues. The term “affinity modified IgSF domain” is not be construed as imposing any condition for any particular starting composition or method by which the affinity-modified IgSF domain was created. Thus, the affinity modified IgSF domains of the present invention are not limited to wild type IgSF domains that are then transformed to an affinity modified IgSF domain by any particular process of affinity modification. An affinity modified IgSF domain polypeptide can, for example, be generated starting from wild type mammalian IgSF domain sequence information, then modeled in silico for binding to its cognate binding partner, and finally recombinantly or chemically synthesized to yield the affinity modified IgSF domain composition of matter. In but one alternative example, an affinity modified IgSF domain can be created by site-directed mutagenesis of a wild-type IgSF domain. Thus, affinity modified IgSF domain denotes a product and not necessarily a product produced by any given process. A variety of techniques including recombinant methods, chemical synthesis, or combinations thereof, may be employed.

The term “allogeneic” as used herein means a cell or tissue that is removed from one organism and then infused or adoptively transferred into a genetically dissimilar organism of the same species.

The term “autologous” as used herein means a cell or tissue that is removed from the same organism to which it is later infused or adoptively transferred. An autologous cell or tissue can be altered by, for example, recombinant DNA methodologies, such that it is no longer genetically identical to the native cell or native tissue which is removed from the organism. For example, a native autologous T-cell can be genetically engineered by recombinant DNA techniques to become an autologous engineered cell expressing a transmembrane immunomodulatory protein and/or chimeric antigen receptor (CAR), which in some cases involves engineering a T-cell or TIL (tumor infiltrating lymphocyte). The engineered cell can then be infused into a patient from which the native T-cell was isolated. In some embodiments, the organism is human or murine.

The terms “binding affinity,” and “binding avidity” as used herein means the specific binding affinity and specific binding avidity, respectively, of a protein for its cognate binding partner (i.e., its counter-structure) under specific binding conditions. In biochemical kinetics avidity refers to the accumulated strength of multiple affinities of individual non-covalent binding interactions, such as between an IgSF domain and its cognate binding partner (i.e., its counter-structure). As such, avidity is distinct from affinity, which describes the strength of a single interaction. An increase or attenuation in binding affinity of an affinity modified IgSF domain to its counter-structure is determined relative to the binding affinity of the unmodified IgSF domain (e.g., the native or wild-type IgSF domain). Methods for determining binding affinity or avidity are known in art. See, for example, Larsen et al., American Journal of Transplantation, Vol 5: 443-453 (2005).

The term “cell surface counter-structure” (alternatively “cognate cell surface binding partner”) as used herein is a counter-structure (alternatively is a cognate binding partner) expressed on a mammalian cell. Typically, the cell surface counter-structure is a transmembrane protein. In some embodiments, the cell surface counter-structure is a receptor.

The term “chimeric antigen receptor” or “CAR” as used herein refers to an artificial (i.e., man-made) transmembrane protein expressed on a mammalian cell comprising at least an ectodomain, a transmembrane, and an endodomain. Optionally, the CAR protein includes a “spacer” which covalently links the ectodomain to the transmembrane domain. A spacer is often a polypeptide linking the ectodomain to the transmembrane domain via peptide bonds. The CAR is typically expressed on a mammalian lymphocyte. In some embodiments, the CAR is expressed on a mammalian cell such as a T-cell or a tumor infiltrating lymphocyte (TIL). A CAR expressed on a T-cell is referred to herein as a CAR T-cell or “CAR-T.” In some embodiments the CAR-T is a T helper cell, a cytotoxic T-cell, a natural killer T-cell, a memory T-cell, a regulatory T-cell, or a gamma delta T-cell. When used clinically in, e.g. adoptive cell transfer, a CAR with antigen binding specificity to the patient's tumor is typically engineered to be expressed on a native lymphocyte obtained from the patient. The engineered lymphocyte expressing the CAR is then infused back into the patient. The lymphocyte is thus often an autologous T-cell although allogeneic T-cells are included within the scope of the invention. The ectodomain of a CAR comprises an antigen binding region, such as an antibody or antigen binding fragment thereof (e.g. scFv), that specifically binds under physiological conditions with an antigen, such as a tumor specific antigen. Upon specific binding a biochemical chain of events (i.e., signal transduction) results in modulation of the immunological activity of the cell on which the CAR is expressed. Thus, for example, upon specific binding by the antigen binding region of the CAR-T to its antigen can lead to changes in the immunological activity of the T-cell activity as reflected by changes in cytotoxicity, proliferation or cytokine production. Signal transduction upon CAR activation is achieved in some embodiments by the CD3-zeta chain (“CD3-z”) which is involved in signal transduction in native mammalian T-cells. CARs can further comprise multiple signaling domains such as CD28, ICOS, 41BB or OX40, to further modulate immunomodulatory response of the T-cell. CD3-z comprises a conserved motif known as an immunoreceptor tyrosine-based activation motif (ITAM) which is involved in T-cell receptor signal transduction.

The terms “cognate binding partner” or “counter-structure” in reference to a protein, such as an IgSF domain or an affinity modified IgSF domain, refers to at least one molecule (typically a native mammalian protein) to which the referenced protein specifically binds under specific binding conditions. In some aspects, an affinity modified IgSF domain specifically binds to the counter-structure of the corresponding native or wildtype IgSF domain but with increased or attenuated affinity. A species of ligand recognized and specifically binding to its cognate receptor under specific binding conditions is an example of a counter-structure or cognate binding partner of that receptor. A receptor, to which a native ligand recognizes and specifically binds to under specific binding conditions, is an example of a counter-structure of that ligand. In turn, the native ligand is the counter-structure of the receptor. For example, ICOSL specifically binds to CD28 and ICOS and thus these proteins are counter-structures of ICOSL. In another example, a tumor specific antigen and an affinity modified IgSF domain to which it specifically binds, are each counter-structures of the other. In the present invention a “cell surface molecular species” is a cognate binding partner of ligands of the immunological synapse (IS), expressed on and by cells, such as mammalian cells, forming the immunological synapse, for example immune cells.

The term “competitive binding” as used herein means that a protein is capable of specifically binding to at least two cognate binding partners but that specific binding of one cognate binding partner inhibits, such as prevents or precludes, simultaneous binding of the second cognate binding partner. Thus, in some cases, it is not possible for a protein to bind the two cognate binding partners at the same time. Generally, competitive binders contain the same or overlapping binding site for binding but this is not a requirement. In some embodiments, competitive binding causes a measurable inhibition (partial or complete) of specific binding of a protein to one of its cognate binding partner due to specific binding of a second cognate binding partner. A variety of methods are known to quantify competitive binding such as ELISA (enzyme linked immunosorbent assay) or Forte-Bio Octet experimental systems.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. By aligning the sequences, one skilled in the art can identify corresponding residues, for example, using conserved and identical amino acid residues as guides.

The term “cytokine” includes, e.g., but is not limited to, interleukins, interferons (IFN), chemokines, hematopoietic growth factors, tumor necrosis factors (TNF), and transforming growth factors. In general, these are small molecular weight proteins that regulate maturation, activation, proliferation, and differentiation of cells of the immune system.

The terms “derivatives” or “derivatized” refer to modification of an immunomodulatory protein by covalently linking it, directly or indirectly, so as to alter such characteristics as half-life, bioavailability, immunogenicity, solubility, toxicity, potency, or efficacy while retaining or enhancing its therapeutic benefit. Derivatives can be made by glycosylation, pegylation, lipidation, or Fc-fusion.

As used herein, “domain” (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule, such as a cognate binding partner. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed appropriate software can be employed to identify domains. It is understood that reference to amino acids, including to a specific sequence set forth as a SEQ ID NO used to describe domain organization of an IgSF domain are for illustrative purposes and are not meant to limit the scope of the embodiments provided. It is understood that polypeptides and the description of domains thereof are theoretically derived based on homology analysis and alignments with similar molecules. Thus, the exact locus can vary, and is not necessarily the same for each protein. Hence, the specific IgSF domain, such as specific IgV domain or IgC domain, can be several amino acids (one, two, three or four) longer or shorter.

The term “ectodomain” as used herein refers to the region of a membrane protein, such as a transmembrane protein, that lies outside the vesicular membrane (e.g., the space outside of a cell). Ectodomains often interact with specific ligands or specific cell surface receptors, such as via a binding domain that specifically binds to the ligand or cell surface receptor. The ectodomain of a cellular transmembrane protein is alternately referred to as an extracellular domain.

The terms “effective amount” or “therapeutically effective amount” refer to a quantity and/or concentration of a therapeutic composition of the invention, such as composition containing engineered cells, that when administered ex vivo (by contact with a cell from a patient) or in vivo (by administration into a patient, such as by adoptive transfer) either alone (i.e., as a monotherapy) or in combination with additional therapeutic agents, yields a statistically significant inhibition of disease progression as, for example, by ameliorating or eliminating symptoms and/or the cause of the disease. An effective amount for treating an immune system disease or disorder may be an amount that relieves, lessens, or alleviates at least one symptom or biological response or effect associated with the disease or disorder, prevents progression of the disease or disorder, or improves physical functioning of the patient. In the case of cell therapy, the effective amount is an effective dose or number of cells administered to a patient. In some embodiments the patient is a human patient.

The term “endodomain” as used herein refers to the region found in some membrane proteins, such as transmembrane proteins, that extends into the interior space defined by the cell surface membrane. In mammalian cells, the endodomain is the cytoplasmic region of the membrane protein. In cells, the endodomain interacts with intracellular constituents and can be play a role in signal transduction and thus, in some cases, can be an intracellular signaling domain. The endodomain of a cellular transmembrane protein is alternately referred to as a cytoplasmic domain, which, in some cases, can be a cytoplasmic signaling domain.

The term “enhanced” or “increased” as used herein in the context of increasing immunological activity of a mammalian lymphocyte means to increase one or more activities of the lymphocyte. An increased activity can be one or more of an increase cell survival, cell proliferation, cytokine production, or T-cell cytotoxicity, such as by a statistically significant amount. In some embodiments, reference to increased immunological activity means to increase interferon gamma (IFN-gamma) production, such as by a statistically significant amount. Methods of assessing activities of lymphocytes are known in the art, including any assay as described herein. In some embodiments an enhancement can be an increase of at least 10%, 20%, 30%, 40%, 50%, 75%, 100%, 200%, 300%, 400%, or 500% greater than a non-zero control value.

The term “engineered cell” as used herein refers to a mammalian cell that has been genetically modified by human intervention such as by recombinant DNA methods or viral transduction. In some embodiments, the cell is an immune cells, such as a lymphocyte (e.g. T cell, B cell, NK cell) or an antigen presenting cell (e.g. dendritic cell). The cell can be a primary cell from a patient or can be a cell line. In the context of the present disclosure, an engineered cell comprises a transmembrane immunomodulatory protein (TIP) as described herein that is expressed on the cell and is engineered to modulate immunological activity of the engineered cell itself, or a mammalian cell to which an affinity modified IgSF domain of the TIP specifically binds. In some cases, the TIP is formatted as a chimeric receptor containing a heterologous cytoplasmic signaling domain or endodomain. Among provided TIP-engineered cells also are cells further containing an engineered T-cell receptor (TCR) or chimeric antigen receptor (CAR).

The term “engineered T-cell” as used herein refers to a T-cell such as a T helper cell, cytotoxic T-cell (alternatively, cytotoxic T lymphocyte or CTL), natural killer T-cell, regulatory T-cell, memory T-cell, or gamma delta T-cell, that has been genetically modified by human intervention such as by recombinant DNA methods. An engineered T-cell comprises a transmembrane immunomodulatory protein (TIP) of the present invention that is expressed on the T-cell and is engineered to modulate immunological activity of the engineered T-cell itself, or a mammalian cell to which an affinity modified IgSF domain of the TIP expressed on the T-cell specifically binds.

The term “engineered T-cell receptor” or “engineered TCR” refers to a T-cell receptor (TCR) engineered to specifically bind with a desired affinity to a major histocompatibility complex (MHC)/peptide target antigen that is selected, cloned, and/or subsequently introduced into a population of T-cells, often used for adoptive immunotherapy. In contrast to engineered TCRs, CARs are engineered to bind target antigens in a MHC independent manner.

The term “expressed on” as used herein is used in reference to a protein expressed on the surface of a cell, such as a mammalian cell. Thus, the protein is expressed as a membrane protein. In some embodiments, the expressed protein is a transmembrane protein. In some embodiments, the protein is conjugated to a small molecule moiety such as a drug or detectable label. Proteins expressed on the surface of a cell can include cell-surface proteins such as cell surface receptors that are expressed on mammalian cells.

The term “host cell” refers to a cell that can be used to express a protein encoded by a recombinant expression vector. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO and related cell lines which grow in serum-free media or CHO strain DX-B11, which is deficient in DHFR.

The term “immunological synapse” or “immune synapse” as used herein means the interface between a mammalian cell that expresses MHC I (major histocompatibility complex) or MHC II, such as an antigen-presenting cell or tumor cell, and a mammalian lymphocyte such as an effector T cell or Natural Killer (NK) cell.

The term “immunoglobulin” (abbreviated “Ig”) as used herein is synonymous with the term “antibody” (abbreviated “Ab”) and refers to a mammalian immunoglobulin protein including any of the five human classes: IgA (which includes subclasses IgA1 and IgA2), IgD, IgE, IgG (which includes subclasses IgG1, IgG2, IgG3, and IgG4), and IgM. The term is also inclusive of immunoglobulins that are less than full-length, whether wholly or partially synthetic (e.g., recombinant or chemical synthesis) or naturally produced, such as antigen binding fragment (Fab), variable fragment (Fv) containing VH and VL, the single chain variable fragment (scFv) containing VH and VL linked together in one chain, as well as other antibody V region fragments, such as Fab′, F(ab)2, F(ab′)2, dsFv diabody, Fc, and Fd polypeptide fragments. Bispecific antibodies, homobispecific and heterobispecific, are included within the meaning of the term.

An Fc (fragment crystallizable) region or domain of an immunoglobulin molecule (also termed an Fc polypeptide) corresponds largely to the constant region of the immunoglobulin heavy chain, and is responsible for various functions, including the antibody's effector function(s). An immunoglobulin Fc fusion (“Fc-fusion”) is a molecule comprising one or more polypeptides (or one or more small molecules) operably linked to an Fc region of an immunoglobulin. An Fc-fusion may comprise, for example, the Fc region of an antibody (which, in some cases, facilitates effector functions and pharmacokinetics) and the IgSF domain of a wild-type or affinity-modified immunoglobulin superfamily domain (“IgSF”), or other protein or fragment thereof. In some embodiments, the Fc is a variant Fc that exhibits reduced (e.g. reduced greater than 30%, 40%, 50%, 60%, 70%, 80%, 90% or more) activity to facilitate an effector function. The IgSF domain mediates recognition of the cognate binding partner (comparable to that of antibody variable region of an antibody for an antigen). An immunoglobulin Fc region may be linked indirectly or directly to one or more polypeptides or small molecules (fusion partners). Various linkers are known in the art and can be used to link an Fc to a fusion partner to generate an Fc-fusion. An Fc-fusion protein of the invention typically comprises an immunoglobulin Fc region covalently linked, directly or indirectly, to at least one affinity modified IgSF domain. Fc-fusions of identical species can be dimerized to form Fc-fusion homodimers, or using non-identical species to form Fc-fusion heterodimers.

The term “immunoglobulin superfamily” or “IgSF” as used herein means the group of cell surface and soluble proteins that are involved in the recognition, binding, or adhesion processes of cells. Molecules are categorized as members of this superfamily based on shared structural features with immunoglobulins (i.e., antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system. Proteins in the immunological synapse are often members of the IgSF. IgSF can also be classified into “subfamilies” based on shared properties such as function. Such subfamilies typically consist of from 4 to 30 IgSF members.

The terms “IgSF domain” or “immunoglobulin domain” or “Ig domain” as used herein refers a structural domain of IgSF proteins. Ig domains are named after the immunoglobulin molecules. They contain about 70-110 amino acids and are categorized according to their size and function. Ig-domains possess a characteristic Ig-fold, which has a sandwich-like structure formed by two sheets of antiparallel beta strands. Interactions between hydrophobic amino acids on the inner side of the sandwich and highly conserved disulfide bonds formed between cysteine residues in the B and F strands, stabilize the Ig-fold. One end of the Ig domain has a section called the complementarity determining region that is important for the specificity of antibodies for their ligands. The Ig like domains can be classified (into classes) as: IgV, IgC1, IgC2, or IgI. Most Ig domains are either variable (IgV) or constant (IgC). IgV domains with 9 beta strands are generally longer than IgC domains with 7 beta strands. Ig domains of some members of the IgSF resemble IgV domains in the amino acid sequence, yet are similar in size to IgC domains. These are called IgC2 domains, while standard IgC domains are called IgC1 domains. T-cell receptor (TCR) chains contain two Ig domains in the extracellular portion; one IgV domain at the N-terminus and one IgC1 domain adjacent to the cell membrane.

The term “IgSF species” as used herein means an ensemble of IgSF member proteins with identical or substantially identical primary amino acid sequence. Each mammalian immunoglobulin superfamily (IgSF) member defines a unique identity of all IgSF species that belong to that IgSF member. Thus, each IgSF family member is unique from other IgSF family members and, accordingly, each species of a particular IgSF family member is unique from the species of another IgSF family member. Nevertheless, variation between molecules that are of the same IgSF species may occur owing to differences in post-translational modification such as glycosylation, phosphorylation, ubiquitination, nitrosylation, methylation, acetylation, and lipidation. Additionally, minor sequence differences within a single IgSF species owing to gene polymorphisms constitute another form of variation within a single IgSF species as do wild type truncated forms of IgSF species owing to, for example, proteolytic cleavage. A “cell surface IgSF species” is an IgSF species expressed on the surface of a cell, generally a mammalian cell.

The term “immunological activity” as used herein in the context of mammalian lymphocytes refers to one or more cell survival, cell proliferation, cytokine production (e.g. interferon-gamma), or T-cell cytotoxicity activities. Methods to assay the immunological activity of engineered cells, including to evaluate the activity of a transmembrane immunomodulatory protein, are known in the art and include, but are not limited to, the ability to expand T cells following antigen stimulation, sustain T cell expansion in the absence of re-stimulation, and anti-cancer activities in appropriate animal models. Assays also include assays to assess cytotoxicity, including a standard ⁵¹Cr-release assay (see e.g. Milone et al., (2009) Molecular Therapy 17: 1453-1464) or flow based cytotoxicity assays, or an impedance based cytotoxicity assay (Peper et al. (2014) Journal of Immunological Methods, 405:192-198). Assays to assess immunological activity of engineered cells can be compared to control non-engineered cells or to cells containing one or more other engineered recombinant receptor (e.g. antigen receptor) with a known activity.

An “immunomodulatory protein” is a protein that modulates immunological activity. By “modulation” or “modulating” an immune response is meant that immunological activity is either enhanced or suppressed. An immunomodulatory protein can be a single polypeptide chain or a multimer (dimers or higher order multimers) of at least two polypeptide chains covalently bonded to each other by, for example, interchain disulfide bonds. Thus, monomeric, dimeric, and higher order multimeric proteins are within the scope of the defined term. Multimeric proteins can be homomultimeric (of identical polypeptide chains) or heteromultimeric (of different polypeptide chains). Transmembrane immunomodulatory proteins are a type of immunomodulatory protein.

The term “increase” as used herein means to increase by a statistically significant amount. An increase can be at least 5%, 10%, 20%, 30%, 40%, 50%, 75%, 100%, or greater than a non-zero control value.

The term “lymphocyte” as used herein means any of three subtypes of white blood cell in a mammalian immune system. They include natural killer cells (NK cells) (which function in cell-mediated, cytotoxic innate immunity), T cells (for cell-mediated, cytotoxic adaptive immunity), and B cells (for humoral, antibody-driven adaptive immunity). T cells include T helper cells, cytotoxic T-cells, natural killer T-cells, memory T-cells, regulatory T-cells, or gamma delta T-cells. Innate lymphoid cells (ILC) are also included within the definition of lymphocyte.

An “inhibitory counter-structure” is a cell membrane protein, often a receptor, which when proximally bound near a separate activating receptor leads to an attenuation in the frequency, duration, magnitude, or intensity of the activating signaling cascade and phenotype mediated by the activating receptor. Examples of inhibitory receptors include PD-1, CTLA-4, LAG-3, TIGIT, CD96, CD112R, BTLA, CD160 and TIM-3. The term “stimulatory counter-structure” is a cell membrane protein, often a receptor, which when activated and signal transduction is thereby induced, leads to an increase in the frequency, duration, or intensity of the phenotype mediated by that receptor. Examples of stimulatory receptors include CD28, ICOS, and CD226.

The term “lymphocyte” as used herein means any of three subtypes of white blood cell in a mammalian immune system. They include natural killer cells (NK cells) (which function in cell-mediated, cytotoxic innate immunity), T cells (for cell-mediated, cytotoxic adaptive immunity), and B cells (for humoral, antibody-driven adaptive immunity). T cells include: T helper cells, cytotoxic T-cells, natural killer T-cells, memory T-cells, regulatory T-cells, or gamma delta T-cells. Innate lymphoid cells (ILC) are also included within the definition of lymphocyte.

The terms “mammal,” “subject,” or “patient” specifically includes reference to at least one of a: human, chimpanzee, rhesus monkey, cynomolgus monkey, dog, cat, mouse, or rat.

The term “membrane protein” as used herein means a protein that, under physiological conditions, is attached directly or indirectly to a lipid bilayer. A lipid bilayer that forms a membrane can be a biological membrane such as a eukaryotic (e.g., mammalian) cell membrane or an artificial (i.e., man-made) membrane such as that found on a liposome. Attachment of a membrane protein to the lipid bilayer can be by way of covalent attachment, or by way of non-covalent interactions such as hydrophobic or electrostatic interactions. A membrane protein can be an integral membrane protein or a peripheral membrane protein. Membrane proteins that are peripheral membrane proteins are non-covalently attached to the lipid bilayer or non-covalently attached to an integral membrane protein. A peripheral membrane protein forms a temporary attachment to the lipid bilayer such that under the range of conditions that are physiological in a mammal, peripheral membrane protein can associate and/or disassociate from the lipid bilayer. In contrast to peripheral membrane proteins, integral membrane proteins form a substantially permanent attachment to the membrane's lipid bilayer such that under the range of conditions that are physiological in a mammal, integral membrane proteins do not disassociate from their attachment to the lipid bilayer. A membrane protein can form an attachment to the membrane by way of one layer of the lipid bilayer (monotopic), or attached by way of both layers of the membrane (polytopic). An integral membrane protein that interacts with only one lipid bilayer is an “integral monotopic protein”. An integral membrane protein that interacts with both lipid bilayers is an “integral polytopic protein” alternatively referred to herein as a “transmembrane protein”.

The terms “modulating” or “modulate” as used herein in the context of an immune response, such as a mammalian immune response, refer to any alteration, such as an increase or decrease, of an existing or potential immune responses that occurs as a result of administration of an immunomodulatory protein or as a result of administration of engineered cells expressing an immunomodulatory protein, such as atransmembrane immunomodulatory protein of the present invention. Such modulation includes any induction, or alteration in degree or extent, or suppression of immunological activity of an immune cell. Immune cells include B cells, T cells, NK (natural killer) cells, NK T cells, professional antigen-presenting cells (APCs), and non-professional antigen-presenting cells, and inflammatory cells (neutrophils, macrophages, monocytes, eosinophils, and basophils). Modulation includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response. Modulation of an immune response or modulation of immunological activity includes, for example, the following: elimination, deletion, or sequestration of immune cells; proliferation, induction, survival or generation of immune cells that can modulate the functional capacity of other cells such as autoreactive lymphocytes, antigen presenting cells, or inflammatory cells; induction of an unresponsive state in immune cells (i.e., anergy); enhancing or suppressing the activity or function of immune cells, including but not limited to altering the pattern of proteins expressed by these cells. Examples include altered production and/or secretion of certain classes of molecules such as cytokines, chemokines, perforins, granzymes, growth factors, transcription factors, kinases, costimulatory molecules, or other cell surface receptors or any combination of these modulatory events. Modulation can be assessed, for example, by an alteration of an immunological activity of engineered cells, such as an alteration in cytotoxic activity of engineered cells or an alteration in cytokine secretion of engineered cells relative to cells engineered with the wild-type IgSF protein.

The term “molecular species” as used herein means an ensemble of proteins with identical or substantially identical primary amino acid sequence. Each mammalian immunoglobulin superfamily (IgSF) member defines a collection of identical or substantially identical molecular species. Thus, for example, human CD80 is an IgSF member and each human CD80 molecule is a species of CD80. Variation between molecules that are of the same molecular species may occur owing to differences in post-translational modification such as glycosylation, phosphorylation, ubiquitination, nitrosylation, methylation, acetylation, and lipidation. Additionally, minor sequence differences within a single molecular species owing to gene polymorphisms constitute another form of variation within a single molecular species as do wild type truncated forms of a single molecular species owing to, for example, proteolytic cleavage. A “cell surface molecular species” is a molecular species expressed on the surface of a mammalian cell. Two or more different species of protein, each of which is present exclusively on one or exclusively the other (but not both) of the two mammalian cells forming the IS, are said to be in “cis” or “cis configuration” with each other. Two different species of protein, the first of which is exclusively present on one of the two mammalian cells forming the IS and the second of which is present exclusively on the second of the two mammalian cells forming the IS, are said to be in “trans” or “trans configuration.” Two different species of protein each of which is present on both of the two mammalian cells forming the IS are in both cis and trans configurations on these cells.

The term “non-competitive binding” as used herein means the ability of a protein to specifically bind simultaneously to at least two cognate binding partners. In some embodiments, the binding occurs under specific binding conditions. Thus, the protein is able to bind to at least two different cognate binding partners at the same time although the binding interaction need not be for the same duration such that, in some cases, the protein is specifically bound to only one of the cognate binding partners. In some embodiments, the simultaneous binding is such that binding of one cognate binding partner does not substantially inhibit simultaneous binding to a second cognate binding partner. In some embodiments, non-competitive binding means that binding a second cognate binding partner to its binding site on the protein does not displace the binding of a first cognate binding partner to its binding site on the protein. Methods of assessing non-competitive binding are well known in the art such as the method described in Perez de La Lastra et al., Immunology, 1999 April: 96(4): 663-670. In some cases, in non-competitive interactions, the first cognate binding partner specifically binds at an interaction site that does not overlap with the interaction site of the second cognate binding partner such that binding of the second cognate binding partner does not directly interfere with the binding of the first cognate binding partner. Thus, any effect on binding of the cognate binding partner by the binding of the second cognate binding partner is through a mechanism other than direct interference with the binding of the first cognate binding partner. For example, in the context of enzyme-substrate interactions, a non-competitive inhibitor binds to a site other than the active site of the enzyme. Non-competitive binding encompasses uncompetitive binding interactions in which a second cognate binding partner specifically binds at an interaction site that does not overlap with the binding of the first cognate binding partner but binds to the second interaction site only when the first interaction site is occupied by the first cognate binding partner.

The terms “nucleic acid” and “polynucleotide” are used interchangeably to refer to a polymer of nucleic acid residues (e.g., deoxyribonucleotides or ribonucleotides) in either single- or double-stranded form. Unless specifically limited, the terms encompass nucleic acids containing known analogues of natural nucleotides and that have similar binding properties to it and are metabolized in a manner similar to naturally-occurring nucleotides. Unless otherwise indicated, a particular nucleic acid sequence also implicitly encompasses conservatively modified variants thereof (e.g., degenerate codon substitutions) and complementary nucleotide sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid or polynucleotide encompasses cDNA or mRNA encoded by a gene.

The term “pharmaceutical composition” refers to a composition suitable for pharmaceutical use in a mammalian subject, often a human. A pharmaceutical composition typically comprises an effective amount of an active agent (e.g., an immunomodulatory protein or engineered cells expressing a transmembrane immunomodulatory protein of the present invention) and a carrier, excipient, or diluent. The carrier, excipient, or diluent is typically a pharmaceutically acceptable carrier, excipient or diluent, respectively.

The terms “polypeptide” and “protein” are used interchangeably herein and refer to a molecular chain of two or more amino acids linked through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. The terms also include molecules in which one or more amino acid analogs or non-canonical or unnatural amino acids are included as can be synthesized, or expressed recombinantly using known protein engineering techniques. In addition, proteins can be derivatized as described herein by well-known organic chemistry techniques.

The term “primary T-cell assay” as used herein refers to an in vitro assay to measure interferon-gamma (“IFN-gamma”) expression. A variety of such primary T-cell assays are known in the art such as that described in Example 6. In a preferred embodiment, the assay used is anti-CD3 coimmobilization assay. In this assay, primary T cells are stimulated by anti-CD3 immobilized with or without additional recombinant proteins. Culture supernatants are harvested at timepoints, usually 24-72 hours. In another embodiment, the assay used is a mixed lymphocyte reaction (MLR). In this assay, primary T cells are simulated with allogenic APC. Culture supernatants are harvested at timepoints, usually 24-72 hours. Human IFN-gamma levels are measured in culture supernatants by standard ELISA techniques. Commercial kits are available from vendors and the assay is performed according to manufacturer's recommendation.

The term “purified” as applied to nucleic acids, such as encoding atransmembrane immunomodulatory proteins, or proteins (e.g. immunomodulatory proteins) generally denotes a nucleic acid or polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid, or protein is at least about 50% pure, usually at least about 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a “recombinant nucleic acid” is one that is made by recombining nucleic acids, e.g., during cloning, affinity modification, DNA shuffling or other well-known molecular biological procedures. A “recombinant DNA molecule,” is comprised of segments of DNA joined together by means of such molecular biological techniques. The term “recombinant protein” or “recombinant polypeptide” as used herein refers to a protein molecule (e.g., an immunomodulatory protein) which is expressed using a recombinant DNA molecule. A “recombinant host cell” is a cell that contains and/or expresses a recombinant nucleic acid or that is otherwise altered by genetic engineering, such as by introducing into the cell a nucleic acid molecule encoding a recombinant protein, such as a transmembrane immunomodulatory protein provided herein. Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. The terms “in operable combination,” “in operable order” and “operably linked” as used herein refer to the linkage of nucleic acid sequences in such a manner or orientation that a nucleic acid molecule capable of directing the transcription of a given gene and/or the synthesis of a desired protein molecule is produced. The term also refers to the linkage of amino acid sequences in such a manner so that a functional protein is produced and/or transported.

The term “recombinant expression vector” as used herein refers to a DNA molecule containing a desired coding sequence (e.g., an immunomodulatory nucleic acid, a transmembrane immunomodulatory nucleic acid) and appropriate nucleic acid sequences necessary for the expression of the operably linked coding sequence in a particular cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the recombinant expression vector, operably linked to the coding sequence so that the expressed protein can be secreted by the recombinant host cell, for more facile isolation of the fusion protein from the cell, if desired. The term includes the vector as a self-replicating nucleic acid structure as well as the vector incorporated into the genome of a host cell into which it has been introduced. Among the vectors are viral vectors, such as lentiviral vectors.

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. Methods for the alignment of sequences for comparison are well known in the art, such methods include GAP, BESTFIT, BLAST, FASTA and TFASTA. The BLAST algorithm calculates percent sequence identity and performs a statistical analysis of the similarity between the two sequences. The software for performing BLAST analysis is publicly available through the National Center for Biotechnology Information (NCBI) website.

The term “soluble” as used herein in reference to proteins, means that the protein is not a membrane protein. In general, a soluble protein contains only the extracellular domain of an IgSF family member receptor, or a portion thereof containing an IgSF domain or domains or specific-binding fragments thereof.

The term “species” as used herein in the context of a nucleic acid sequence or a polypeptide sequence refers to an identical collection of such sequences. Slightly truncated sequences that differ (or encode a difference) from the full length species at the amino-terminus or carboxy-terminus by no more than 1, 2, or 3 amino acid residues are considered to be of a single species. Such microheterogeneities are a common feature of manufactured proteins.

The term “specifically binds” as used herein means the ability of a protein, under specific binding conditions, to bind to a target protein such that its affinity or avidity is at least 10 times as great, but optionally 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule (e.g., its cognate binding partner) but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a non-target molecule having a substantially similar epitope as the target molecule (e.g., paralog) is possible and does not detract from the specificity of binding which is determined relative to a statistically valid collection of unique non-targets (e.g., random polypeptides). Thus, an affinity-modified polypeptide of the invention may specifically bind to more than one distinct species of target molecule due to cross-reactivity. Generally, such off-target specific binding is mitigated by reducing affinity or avidity for undesired targets. Solid-phase ELISA immunoassays or Biacore measurements can be used to determine specific binding between two proteins. Generally, interactions between two binding proteins have dissociation constants (Kd) less than 1×10-5 M, and often as low as 1×10-12 M. In certain aspects of the present disclosure, interactions between two binding proteins have dissociation constants of 1×10-6 M, 1×10-7 M, 1×10-8 M, 1×10-9 M, 1×10-10 M or 1×10-11 M.

The term “specific binding fragment” or “fragment” as used herein in reference to a mature (i.e., absent the signal peptide) wild-type IgSF domain, means a polypeptide that is shorter than the full-length mature IgSF domain and that specifically binds in vitro and/or in vivo to the wild-type IgSF domain's native cognate binding partner. In some embodiments, the specific binding fragment is at at least 20%, 30%, 40%, 50%, 60%, 70%, 75%, 80%, 85%, 90%, 95%, 96%, 97%, 98%, or 99% the sequence length of the full-length mature wild-type sequence. The specific binding fragment can be altered in sequence to form an affinity modified IgSF domain of the invention. In some embodiments, the specific binding fragment modulates immunological activity of a lymphocyte. The term “suppress” or “attenuate” or “decrease” as used herein means to decrease by a statistically significant amount. In some embodiments suppression can be a decrease of at least 10%, and up to 20%, 30%, 40%, 50%, 60%, 70%, 80%, or 90%.

The terms “surface expresses” or “surface expression” in reference to a mammalian cell expressing a polypeptide means that the polypeptide is expressed as a membrane protein. In some embodiments, the membrane protein is a transmembrane protein.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

The term “transmembrane protein” as used herein means a membrane protein that substantially or completely spans a lipid bilayer such as those lipid bilayers found in a biological membrane such as a mammalian cell, or in an artificial construct such as a liposome. The transmembrane protein comprises a transmembrane domain (“transmembrane domain”) by which it is integrated into the lipid bilayer and by which the integration is thermodynamically stable under physiological conditions. Transmembrane domains are generally predictable from their amino acid sequence via any number of commercially available bioinformatics software applications on the basis of their elevated hydrophobicity relative to regions of the protein that interact with aqueous environments (e.g., cytosol, extracellular fluid). A transmembrane domain is often a hydrophobic alpha helix that spans the membrane. A transmembrane protein can pass through the both layers of the lipid bilayer once or multiple times. A transmembrane protein includes the provided transmembrane immunomodulatory proteins described herein. In addition to the transmembrane domain, a transmembrane immunomodulatory protein of the invention further comprises an ectodomain and, in some embodiments, an endodomain.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of an immunomodulatory protein or engineered cells expressing a transmembrane immunomodulatory protein of the present invention either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. As used herein in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS). “Preventing,” “prophylaxis,” or “prevention” of a disease or disorder as used in the context of this invention refers to the administration of an immunomodulatory protein or engineered cells expressing a transmembrane immunomodulatory protein of the present invention, either alone or in combination with another compound, to prevent the occurrence or onset of a disease or disorder or some or all of the symptoms of a disease or disorder or to lessen the likelihood of the onset of a disease or disorder.

The term “tumor specific antigen” or “TSA” as used herein refers to a an antigen that is present primarily on tumor cells of a mammalian subject but generally not found on normal cells of the mammalian subject. In some cases, a tumor specific antigen is a counter structure or cognate binding partner of an IgSF member. A tumor specific antigen need not be exclusive to tumor cells but the percentage of cells of a particular mammal that have the tumor specific antigen is sufficiently high or the levels of the tumor specific antigen on the surface of the tumor are sufficiently high such that it can be targeted by anti-tumor therapeutics and provide prevention or treatment of the mammal from the effects of the tumor. In some embodiments, in a random statistical sample of cells from a mammal with a tumor, at least 50% of the cells displaying a TSA are cancerous. In other embodiments, at least 60%, 70%, 80%, 85%, 90%, 95%, or 99% of the cells displaying a TSA are cancerous.

The term “wild-type” or “natural” or “native” as used herein is used in connection with biological materials such as nucleic acid molecules, proteins, IgSF members, host cells, and the like, refers to those which are found in nature and not modified by human intervention.

### II. TRANSMEMBRANE IMMUNOMODULATORY PROTEINS AND ENGINEERED CELLS

Provided herein are immunomodulatory proteins that are transmembrane proteins (“transmembrane immunomodulatory proteins”). Transmembrane immunomodulatory proteins, and engineered cells expressing such transmembrane immunomodulatory proteins, generally have therapeutic utility by modulating immunological activity in a mammal with a disease or disorder in which modulation of the immune system response is beneficial. A transmembrane immunomodulatory protein of the present invention comprises an ectodomain, a transmembrane, and in some embodiments, an endodomain, such as a cytoplasmic signaling domain.

A. Ectodomain

In some embodiments, the provided transmembrane immunomodulatory proteins include an ectodomain comprising at least one affinity modified IgSF domain compared to an IgSF domain of a wild-type mammalian IgSF member. The wild-type mammalian IgSF member excludes antibodies (i.e., immunoglobulins) such as those that are mammalian or may be of mammalian origin. Thus, the present invention relates to ectodomains that are non-immunoglobulin (i.e., non-antibody) IgSF domains. Wild-type mammalian IgSF family members that are not immunoglobulins (i.e. antibodies) are known in the art as are their nucleic and amino acid sequences. Affinity-modified IgSF domains of a wild-type IgSF domain of all non-immunoglobulin mammalian IgSF family members are included as an ectodomain within the scope of the invention.

In some embodiments, transmembrane immunomodulatory proteins of the present invention in their various embodiments comprise at least one affinity modified mammalian IgSF domain, such as at least one affinity modified non-immunoglobulin mammalian IgSF domain. In some embodiments, the non-immunoglobulin IgSF family members, and the corresponding IgSF domains present therein, are of mouse, rat, cynomolgus monkey, or human origin. In some embodiments, the IgSF family members are members from at least or exactly one, two, three, four, five, or more IgSF subfamilies such as: Signal-Regulatory Protein (SIRP) Family, Triggering Receptor Expressed On Myeloid Cells Like (TREML) Family, Carcinoembryonic Antigen-related Cell Adhesion Molecule (CEACAM) Family, Sialic Acid Binding Ig-Like Lectin (SIGLEC) Family, Butyrophilin Family, B7 family, CD28 family, V-set and Immunoglobulin Domain Containing (VSIG) family, V-set transmembrane Domain (VSTM) family, Major Histocompatibility Complex (MHC) family, Signaling lymphocytic activation molecule (SLAM) family, Leukocyte immunoglobulin-like receptor (LIR), Nectin (Nec) family, Nectin-like (NECL) family, Poliovirus receptor related (PVR) family, Natural cytotoxicity triggering receptor (NCR) family, or Killer-cell immunoglobulin-like receptors (KIR) family. In some embodiments, the at least one IgSF domain is derived from an IgSF protein that is any of CD80(B7-1), CD86(B7-2), CD274 (PD-L1, B7-H1), PDCD1LG2(PD-L2, CD273), ICOSLG(B7RP1, CD275, ICOSL, B7-H2), CD276(B7-H3), VTCN1(B7-H4), CD28, CTLA4, PDCD1(PD-1), ICOS, BTLA(CD272), CD4, CD8A(CD8-alpha), CD8B(CD8-beta), LAG3, HAVCR2(TIM-3), CEACAM1, TIGIT, PVR(CD155), PVRL2(CD112), CD226, CD2, CD160, CD200, CD200R1(CD200R), and NC R3 (NKp30).

In some embodiments, the ectodomain of the transmembrane immunomodulatory protein contains at least one affinity modified IgSF domain. In some embodiments, the at least one affinity-modified IgSF domain is affinity modified compared to a corresponding IgSF domain of a non-immunoglobulin IgSF family member that is a mammalian IgSF member. In some embodiments, the mammalian IgSF member is one of the IgSF members or comprises an IgSF domain from one of the IgSF members as indicated in Table 1 including any mammalian orthologs thereof. Orthologs are genes in different species that evolved from a common ancestral gene by speciation. Normally, orthologs retain the same function in the course of evolution. In some embodiments, the affinity modified IgSF domain is an affinity modified IgV or IgC domain, including IgC1 or IgC2 domain.

In some embodiments, the ectodomain of the transmembrane immunomodulatory protein of the present invention comprises the sequence of the extracellular domain of a wild-type mammalian non-immunoglobulin (i.e., non-antibody) IgSF family member but wherein at least one IgSF domain therein is affinity modified (“Type I” transmembrane immunomodulatory proteins). Additional domains present within the IgSF family can be affinity modified, such as at least two, three, four, or five IgSF domains and, in some embodiments, exactly two, three, four, or five IgSF domains. In some embodiments of a Type I transmembrane immunomodulatory protein of the invention, the mammalian IgSF member will be one of the IgSF members as indicated in Table 1 including any mammalian orthologs thereof

The first column of Table 1 provides the name and, optionally, the name of some possible synonyms for that particular IgSF member. The second column provides the protein identifier of the UniProtKB database, a publicly available database accessible via the internet at uniprot.org. The Universal Protein Resource (UniProt) is a comprehensive resource for protein sequence and annotation data. The UniProt databases include the UniProt Knowledgebase (UniProtKB). UniProt is a collaboration between the European Bioinformatics Institute (EMBL-EBI), the SIB Swiss Institute of Bioinformatics and the Protein Information Resource (PR) and supported mainly by a grant from the U.S. National Institutes of Health (NIH). The third column provides the region where the indicated IgSF domain is located. The region is specified as a range where the domain is inclusive of the residues defining the range. Column 3 also indicates the IgSF domain class for the specified IgSF region. Column 4 provides the region where the indicated additional domains are located (signal peptide, S; extracellular domain, E; transmembrane domain, T; cytoplasmic domain, C). Column 5 indicates for some of the listed IgSF members, some of its cognate cell surface binding partners.

Typically, the affinity modified IgSF domain of the ectodomain of a transmembrane immunomodulatory protein of the provided embodiments is a human or murine affinity modified IgSF domain.

TABLE 1
IgSF members according to the present disclosure.

| | | | | | IgSF Member Amino Acid Sequence | | |
|---|---|---|---|---|---|---|---|
| | | | | Cognate Cell | (SEQ ID NO) | | |
| IgSF | UniProtKB | IgSF Region | | Surface | Precursor | | |
| Member | Protein | & Domain | Other | Binding | (mature | | |
| (Synonyms) | Identifier | Class | Domains | Partners | residues) | Mature | ECD |
| CD80 | NP_005182.1 | 35-138 or | S: 1-34, | CD28, CTLA4, | SEQ ID | SEQ ID | SEQ ID |
| (B7-1) | P33681 | 37-138 IgV, | E: 35-242, | PD-L1 | NO: 1 | NO: 393 | NO: 28 |
| | | 145-230 or | T: 243-263, | | (35-288) | | |
| | | 154-232 IgC | C: 264-288 | | | | |
| CD86 | P42081.2 | 33-131 IgV, | S: 1-23, | CD28, CTLA4 | SEQ ID | SEQ ID | SEQ ID |
| (B7-2) | | 150-225 IgC2 | E: 24-247, | | NO: 2 | NO: 394 | NO: 29 |
| | | | T: 248-268, | | (24-329) | | |
| | | | C: 269-329 | | | | |
| CD274 | Q9NZQ7.1 | 24-130 IgV, | S: 1-18, | PD-1, B7-1 | SEQ ID | SEQ ID | SEQ ID |
| (PD-L1, | | 133-225 IgC2 | E: 19-238, | | NO: 3 | NO: 395 | NO: 30 |
| B7-H1) | | | T: 239-259, | | (19-290) | | |
| | | | C: 260-290 | | | | |
| PDCD1LG2 | Q9BQ51.2 | 21-118 IgV, | S: 1-19, | PD-1, RGMb | SEQ ID | SEQ ID | SEQ ID |
| (PD-L2, | | 122-203 IgC2 | E: 20-220, | | NO: 4 | NO: 396 | NO: 31 |
| CD273) | | | T: 221-241, | | (20-273) | | |
| | | | C: 242-273 | | | | |
| ICOSLG | O75144.2 | 19-129 IgV, | S: 1-18, | ICOS, CD28, | SEQ ID | SEQ ID | SEQ ID |
| (B7RP1, | | 141-227 IgC2 | E: 19-256, | CTLA4 | NO: 5 | NO: 397 | NO: 32 |
| CD275, | | | T: 257-277, | | (19-302) | | |
| ICOSL, | | | C: 278-302 | | | | |
| B7-H2) | | | | | | | |
| CD276 | Q5ZPR3.1 | 29-139 IgV, | S: 1-28, | | SEQ ID | SEQ ID | SEQ ID |
| (B7-H3) | | 145-238 IgC2, | E: 29-466, | | NO: 6 | NO: 398 | NO: 33 |
| | | 243-357 IgV, | T: 467-487, | | (29-534) | | |
| | | 367-453 IgC | C: 488-534 | | | | |
| VTCN1 | Q7Z7D3.1 | 35-146 IgV, | S: 1-24, | | SEQ ID | SEQ ID | SEQ ID |
| (B7-H4) | | 153-241 IgV | E: 25-259, | | NO: 7 | NO: 399 | NO: 34 |
| | | | T: 260-280, | | (25-282) | | |
| | | | C: 281-282 | | | | |
| CD28 | P10747.1 | 28-137 IgV | S: 1-18, | B7-1, B7-2, | SEQ ID | SEQ ID | SEQ ID |
| | | | E: 19-152, | B7RP1 | NO: 8 | NO: 400 | NO: 35 |
| | | | T: 153-179, | | (19-220) | | |
| | | | C: 180-220 | | | | |
| CTLA4 | P16410.3 | 39-140 IgV | S: 1-35, | B7-1, B7-2, | SEQ ID | SEQ ID | SEQ ID |
| | | | E: 36-161, | B7RP1 | NO: 9 | NO: 401 | NO: 36 |
| | | | T: 162-182, | | (36-223) | | |
| | | | C: 183-223 | | | | |
| PDCD1 | Q15116.3 | 35-145 IgV | S: 1-20, | PD-L1, PD-L2 | SEQ ID | SEQ ID | SEQ ID |
| (PD-1) | | | E: 21-170, | | NO: 10 | NO: 402 | NO: 37 |
| | | | T: 171-191, | | (21-288) | | |
| | | | C: 192-288 | | | | |
| ICOS | Q9Y6W8.1 | 30-132 IgV | S: 1-20, | B7RP1 | SEQ ID | SEQ ID | SEQ ID |
| | | | E: 21-140, | | NO: 11 | NO: 403 | NO: 38 |
| | | | T: 141-161, | | (21-199) | | |
| | | | C: 162-199 | | | | |
| BTLA | Q7Z6A9.3 | 31-132 IgV | S: 1-30, | HVEM | SEQ ID | SEQ ID | SEQ ID |
| (CD272) | | | E: 31-157, | | NO: 12 | NO: 404 | NO: 39 |
| | | | T: 158-178, | | (31-289) | | |
| | | | C: 179-289 | | | | |
| CD4 | P01730.1 | 26-125 IgV, | S: 1-25, | MHC class II | SEQ ID | SEQ ID | SEQ ID |
| | | 126-203 IgC2, | E: 26-396, | | NO: 13 | NO: 405 | NO: 40 |
| | | 204-317 IgC2, | T: 397-418, | | (26-458) | | |
| | | 317-389 IgC2 | C: 419-458 | | | | |
| CD8A | P01732.1 | 22-135 IgV | S: 1-21, | MHC class I | SEQ ID | SEQ ID | SEQ ID |
| (CD8- | | | E: 22-182, | | NO: 14 | NO: 406 | NO: 41 |
| alpha) | | | T: 183-203, | | (22-235) | | |
| | | | C: 204-235 | | | | |
| CD8B | P10966.1 | 22-132 IgV | S: 1-21, | MHC class I | SEQ ID | SEQ ID | SEQ ID |
| (CD8- | | | E: 22-170, | | NO: 15 | NO: 407 | NO: 42 |
| beta) | | | T: 171-191, | | (22-210) | | |
| | | | C: 192-210 | | | | |
| LAG3 | P18627.5 | 37-167 IgV, | S: 1-28, | MHC class II | SEQ ID | SEQ ID | SEQ ID |
| | | 168-252 IgC2, | E: 29-450, | | NO: 16 | NO: 408 | NO: 43 |
| | | 265-343 IgC2, | T: 451-471, | | (29-525) | | |
| | | 349-419 IgC2 | C: 472-525 | | | | |
| HAVCR2 | Q8TDQ0.3 | 22-124 IgV | S: 1-21, | CEACAM-1, | SEQ ID | SEQ ID | SEQ ID |
| (TIM-3) | | | E: 22-202, | phosphatidyl- | NO: 17 | NO: 409 | NO: 44 |
| | | | T: 203-223, | serine, | (22-301) | | |
| | | | C: 224-301 | Galectin-9, | | | |
| | | | | HMGB1 | | | |
| CEACAM1 | P13688.2 | 35-142 IgV, | S: 1-34, | TIM-3 | SEQ ID | SEQ ID | SEQ ID |
| | | 145-232 IgC2, | E: 35-428, | | NO: 18 | NO: 410 | NO: 45 |
| | | 237-317 IgC2, | T: 429-452, | | (35-526) | | |
| | | 323-413 IgC | C: 453-526 | | | | |
| TIGIT | Q495A1.1 | 22-124 IgV | S: 1-21, | CD155, CD112 | SEQ ID | SEQ ID | SEQ ID |
| | | | E: 22-141, | | NO: 19 | NO: 411 | NO: 46 |
| | | | T: 142-162, | | (22-244) | | |
| | | | C: 163-244 | | | | |
| PVR | P15151.2 | 24-139 IgV, | S: 1-20, | TIGIT, CD226, | SEQ ID | SEQ ID | SEQ ID |
| (CD155) | | 145-237 IgC2, | E: 21-343, | CD96, | NO: 20 | NO: 412 | NO: 47 |
| | | 244-328 IgC2 | T: 344-367, | poliovirus | (21-417) | | |
| | | | C: 368-417 | | | | |
| PVRL2 | Q92692.1 | 32-156 IgV, | S: 1-31, | TIGIT, CD226, | SEQ ID | SEQ ID | SEQ ID |
| (CD112) | | 162-256 IgC2, | E: 32-360, | CD112R | NO: 21 | NO: 413 | NO: 48 |
| | | 261-345 IgC2 | T: 361-381, | | (32-538) | | |
| | | | C: 382-538 | | | | |
| CD226 | Q15762.2 | 19-126 IgC2, | S: 1-18, | CD155, CD112 | SEQ ID | SEQ ID | SEQ ID |
| | | 135-239 IgC2 | E: 19-254, | | NO: 22 | NO: 414 | NO: 49 |
| | | | T: 255-275, | | (19-336) | | |
| | | | C: 276-336 | | | | |
| CD2 | P06729.2 | 25-128 IgV, | S: 1-24, | CD58 | SEQ ID | SEQ ID | SEQ ID |
| | | 129-209 IgC2 | E: 25-209, | | NO: 23 | NO: 415 | NO: 50 |
| | | | T: 210-235, | | (25-351) | | |
| | | | C: 236-351 | | | | |
| CD160 | O95971.1 | 27-122 IgV | N/A | HVEM, MHC | SEQ ID | SEQ ID | SEQ ID |
| | | | | family of | NO: 24 | NO: 416 | NO: 51 |
| | | | | proteins | (27-159) | | |
| CD200 | P41217.4 | 31-141 IgV, | S: 1-30, | CD200R | SEQ ID | SEQ ID | SEQ ID |
| | | 142-232 IgC2 | E: 31-232, | | NO: 25 | NO: 417 | NO: 52 |
| | | | T: 233-259, | | (31-278) | | |
| | | | C: 260-278 | | | | |
| CD200R1 | Q8TD46.2 | 53-139 IgV, | S: 1-28, | CD200 | SEQ ID | SEQ ID | SEQ ID |
| (CD200R) | | 140-228 IgC2 | E: 29-243, | | NO: 26 | NO: 418 | NO: 53 |
| | | | T: 244-264, | | (29-325) | | |
| | | | C: 265-325 | | | | |
| NCR3 | O14931.1 | 19-126 IgC- | S: 1-18, | B7-H6 | SEQ ID | SEQ ID | SEQ ID |
| (NKp30) | | like | E: 19-135, | | NO: 27 | NO: 419 | NO: 54 |
| | | | T: 136-156, | | (19-201) | | |
| | | | C: 157-201 | | | | |

In some embodiments, the ectodomain of the transmembrane immunomodulatory protein further contains at least one affinity modified domain and further contains at least one non-affinity modified IgSF domain (e.g. unmodified or wildtype IgSF domain). In some embodiments, the ectodomain of the transmembrane immunomodulatory protein contains at least two affinity modified domains. In some embodiments, the ectodomain of the transmembrane immunomodulatory protein can contain a plurality of non-affinity modified IgSF domains and/or affinity modified IgSF domains such as 1, 2, 3, 4, 5, or 6 non-affinity modified IgSF and/or affinity modified IgSF domains.

In some embodiments, the ectodomain of a transmembrane immunomodulatory protein comprises a combination (a “non-wild-type combination”) and/or arrangement (a “non-wild type arrangement” or “non-wild-type permutation”) of an affinity modified and/or non-affinity modified IgSF domain sequences that are not found in wild-type IgSF family members (“Type II” immunomodulatory proteins). The sequences of the IgSF domains which are non-affinity modified (e.g., wild-type) or have been affinity modified can be mammalian, such as from mouse, rat, cynomolgus monkey, or human origin, or combinations thereof. In some embodiments, the sequence of the non-affinity modified domain is any IgSF domain set forth in Table 1. The number of such non-affinity modified or affinity modified IgSF domains present in these embodiments of a Type II immunomodulatory protein (whether non-wild type combinations or non-wild type arrangements) is at least 2, 3, 4, or 5 and in some embodiments exactly 2, 3, 4, or 5 IgSF domains.

In some embodiments, at least two of the affinity modified IgSF domains are identical affinity modified IgSF domains. In some embodiments, the affinity modified IgSF domains are non-identical (i.e., different) IgSF domains. Non-identical affinity modified IgSF domains specifically bind, under specific binding conditions, different cognate binding partners and are “non-identical” irrespective of whether or not the wild-type IgSF domains from which they are designed was the same. Thus, for example, a combination of at least two non-identical IgSF domains in the ectodomain of a transmembrane immunomodulatory protein of the present invention can comprise at least one IgSF domain sequence whose origin is from and unique to one IgSF family member and at least one of a second IgSF domain sequence whose origin is from and unique to another IgSF family member wherein the IgSF domains of the ectodomain of a transmembrane immunomodulatory protein are in affinity modified form. However, in alternative embodiments, the two non-identical IgSF domains originate from the same IgSF domain sequence but are affinity modified differently such that they specifically bind to different cognate binding partners. In some embodiments, the number of non-identical affinity modified IgSF domains present in the ectodomain of a transmembrane immunomodulatory protein of the invention is at least 2, 3, 4, or 5 and in some embodiments exactly 2, 3, 4, or 5 non-identical affinity modified IgSF domains. In some embodiments, the non-identical IgSF domains are combinations from at least two IgSF members indicated in Table 1, and in some embodiments at least three or four IgSF members of Table 1.

In other embodiments an ectodomain of a transmembrane immunomodulatory protein provided herein comprises at least two IgSF domains from a single IgSF member but in a non-wild-type arrangement. One illustrative example of a non-wild type arrangement or permutation is an immunomodulatory protein of the present invention comprising a non-wild type order of affinity modified IgSF domain sequences relative to those found in the wild-type mammalian IgSF family member whose IgSF domain sequences served as the source of the affinity modified IgSF domains. The mammalian wild-type IgSF member in the preceding embodiment specifically includes those listed in Table 1. Thus, in one example, if the wild-type family member comprises an IgC1 domain proximal to the transmembrane domain of a cell surface protein and an IgV domain distal to the transmembrane domain, then the ecotodomain of a transmembrane immunomodulatory protein provided herein can comprise an IgV proximal and an IgC1 distal to the transmembrane domain albeit in affinity modified form. The presence, in an ectodomain of a transmembrane immunomodulatory protein, of both non-wild type combinations and non-wild type arrangements of affinity modified IgSF domains is also within the scope of the present invention. A plurality of affinity modified IgSF domains in an ectodomain of a transmembrane immunomodulatory protein's polypeptide chain need not be covalently linked directly to one another. In some embodiments, an intervening span of one or more amino acid residues indirectly covalently bonds the affinity modified IgSF domains to each other. Such “peptide linkers” can be a single amino acid residue or greater in length.

In some embodiments, the affinity modified IgSF domain can be affinity modified to specifically bind to a single (e.g., 1) or multiple (e.g., 2, 3, 4, or more) counter-structures (also called a “cognate binding partner”) expressed on a mammalian cell. Typically, the counter-structure is a native counter-structure of the wild-type IgSF domain that has been affinity modified. In some embodiments, the counter-structure is an IgSF member. In some embodiments the counter-structure is a non-IgSF family member. For example, in some embodiments the counter-structure of an affinity modified IgSF domain such as BTLA (B- and T-lymphocyte attenuation) is the non-IgSF member counter-structure HVEM (herpes virus entry mediator). BTLA-HVEM complexes negatively regulate T-cell immune responses. Each IgSF domain present in a transmembrane immunomodulatory protein can be affinity modified to independently increase or attenuate specific binding affinity or avidity to each of the single or multiple counter-structures to which it binds. By this method, specific binding to each of multiple counter-structures is independently tuned to a particular affinity or avidity.

In some embodiments, the counter-structure of an IgSF domain is at least one, and sometimes at least two or three of the counter-structures (cognate binding partners) of the wild-type IgSF domain, such as those listed in Table 1. The sequence of the IgSF domain, such as mammalian IgSF domain, is affinity modified by altering its sequence with at least one substitution, addition, or deletion. Alteration of the sequence can occur at the binding site for the counter-structure or at an allosteric site. In some embodiments, a nucleic acid encoding an IgSF domain, such as a mammalian IgSF domain, is affinity modified by substitution, addition, deletion, or combinations thereof, of specific and pre-determined nucleotide sites to yield a nucleic acid encoding an ectodomain of the transmembrane immunomodulatory protein of the invention. In some contrasting embodiments, a nucleic acid encoding an IgSF domain, such as a mammalian IgSF domain, is affinity modified by substitution, addition, deletion, or combinations thereof, at random sites within the nucleic acid. In some embodiments, a combination of the two approaches (pre-determined and random) is utilized. In some embodiments, design of the affinity modified IgSF domains is performed in silico.

In some embodiments, the affinity modified IgSF domain of the ectodomain contains one or more amino acid substitutions (alternatively, “mutations” or “replacements”) relative to a wild-type or unmodified polypeptide or a portion thereof containing an immunoglobulin superfamily (IgSF) domain. In some embodiments, the IgSF domain is an IgV domain or an IgC domain or specific binding fragment of the IgV domain or the IgC domain. In some embodiments, the ectodomain of the transmembrane immunomodulatory protein comprises an affinity modified IgSF domain that contains an IgV domain or an IgC domain or specific binding fragments thereof in which the at least one of the amino acid substitutions is in the IgV domain or IgC domain or a specific binding fragment thereof. In some embodiments, by virtue of the altered binding activity or affinity, the IgV domain or IgC domain is an affinity-modified IgSF domain.

In some embodiments, the IgSF domain, such as a mammalian IgSF domain, is affinity modified in sequence with at least one but no more than a total of 1, 2, 3, 4, 5, 6, 7, 8, 9, or 10 amino acid substitutions, additions, deletions, or combinations thereof. In some embodiments, the IgSF domain, such as mammalian IgSF domain, is affinity modified in sequence with at least one but no more than 10, 9, 8, 7, 6, 5, 4, 3, 2, or 1 amino acid substitutions. In some embodiments, the substitutions are conservative substitutions. In some embodiments, the substitutions are non-conservative. In some embodiments, the substitutions are a combination of conservative and non-conservative substitutions. In some embodiments, the modification in sequence is made at the binding site of the IgSF domain for its counter-structure.

In some embodiments, the wild-type or unmodified IgSF domain is a mammalian IgSF domain. In some embodiments, the wild-type or unmodified IgSF domain can be an IgSF domain that includes, but is not limited to, human, mouse, cynomolgus monkey, or rat. In some embodiments, the wild-type or unmodified IgSF domain is human.

In some embodiments, the wild-type or unmodified IgSF domain is or comprises an extracellular domain of an IgSF family member or a portion thereof containing an IgSF domain (e.g. IgV domain or IgC domain). In some cases, the extracellular domain of an unmodified or wild-type IgSF domain can comprise more than one IgSF domain, for example, an IgV domain and an IgC domain. However, the affinity modified IgSF domain need not comprise both the IgV domain and the IgC domain. In some embodiments, the affinity modified IgSF domain comprises or consists essentially of the IgV domain or a specific binding fragment thereof. In some embodiments, the affinity modified IgSF domain comprises or consists essentially of the IgC domain or a specific binding fragment thereof. In some embodiments, the affinity modified IgSF domain comprises the IgV domain or a specific binding fragment thereof, and the IgC domain or a specific binding fragment thereof.

In some embodiments, the one or more amino acid substitutions of the affinity modified IgSF domain can be located in any one or more of the IgSF polypeptide domains. For example, in some embodiments, one or more amino acid substitutions are located in the extracellular domain of the IgSF polypeptide. In some embodiments, one or more amino acid substitutions are located in the IgV domain or specific binding fragment of the IgV domain. In some embodiments, one or more amino acid substitutions are located in the IgC domain or specific binding fragment of the IgC domain.

In some embodiments, the wild-type or unmodified IgSF domain is an IgSF domain or specific binding fragment thereof contained in the sequence of amino acids set forth in any of SEQ ID NOS:1-27 or contained in a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS:1-27. In some embodiments, the IgSF domain is an IgV domain or IgC domain contained therein or specific binding fragments thereof. Table 1 identifies the IgSF domains contained in each of SEQ ID NOS: 1-27.

In some embodiments, the unmodified or wild-type IgSF domain comprises the extracellular domain (ECD) or a portion comprising an IgSF domain (e.g. IgV domain or IgC domain) of an IgSF member, such as a mammalian IgSF member. In some embodiments, the unmodified or wild-type IgSF domain comprises (i) the sequence of amino acids set forth in any of SEQ ID NOS:28-54, (ii) a sequence of amino acids that has at least about 85%, 86%, 87%, 88%, 89%, 90%, 91%, 92%, 93%, 94%, 95%, 96%, 97%, 98%, 99% sequence identity to any of SEQ ID NOS: 28-54, or (iii) is a specific binding fragment of (i) or (ii) comprising an IgV domain or an IgC domain.

In some embodiments, at least one IgSF domain, such as at least one mammalian IgSF domain, of a transmembrane immunomodulatory protein of the present invention is independently affinity modified in sequence to have at least 99%, 98%, 97%, 96%, 95%, 94%, 93%, 92%, 91%, 90%, 89%, 88%, 87%, 86% 85%, or 80% sequence identity with the corresponding wild-type IgSF domain or specific binding fragment thereof contained in a wild-type or unmodified IgSF protein, such as, but not limited to, those disclosed in Table 1 as SEQ ID NOS: 1-27.

In some embodiments, the affinity-modified IgSF domain of a transmembrane immunomodulatory protein provided herein is a specific binding fragment of a wild-type or unmodified IgSF domain contained in a wild-type or unmodified IgSF protein, such as but not limited to, those disclosed in Table 1 in SEQ ID NOS: 1-27. In some embodiments, the specific binding fragment can have an amino acid length of at least 50 amino acids, such as at least 60, 70, 80, 90, 100, or 110 amino acids. In some embodiments, the specific binding fragment of the IgV domain contains an amino acid sequence that is at least about 85%, 86%, 87%, 88%, 89%, 90%, 91%, 92%, 93%, 94%, 95%, 96%, 97%, 98%, 99% of the length of the wild-type or unmodified IgV domain. In some embodiments, the specific binding fragment of the IgC domain comprises an amino acid sequence that is at least about 85%, 86%, 87%, 88%, 89%, 90%, 91%, 92%, 93%, 94%, 95%, 96%, 97%, 98%, 99% of the length of the wild-type or unmodified IgC domain. In some embodiments, the specific binding fragment modulates immunological activity. In more specific embodiments, the specific binding fragment of an IgSF domain increases immunological activity. In alternative embodiments, the specific binding fragment decreases immunological activity.

In some embodiments, to determine the percent identity of two nucleic acid sequences or of two amino acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length. One may manually align the sequences and count the number of identical nucleic acids or amino acids. Alternatively, alignment of two sequences for the determination of percent identity may be accomplished using a mathematical algorithm. Such an algorithm is incorporated into the NBLAST and XBLAST programs. BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention. To obtain gapped alignments for comparison purposes, Gapped BLAST may be utilized. Alternatively, PSI-Blast may be used to perform an iterated search which detects distant relationships between molecules. When utilizing the NBLAST, XBLAST, and Gapped BLAST programs, the default parameters of the respective programs may be used such as those available on the NCBI website. Alternatively, sequence identity may be calculated after the sequences have been aligned e.g. by the BLAST program in the NCBI database. Generally, the default settings with respect to e.g. “scoring matrix” and “gap penalty” may be used for alignment. In the context of the present invention, the BLASTN and PSI BLAST NCBI default settings may be employed.

The means by which the affinity-modified IgSF domains of the transmembrane immunomodulatory proteins are designed or created is not limited to any particular method. In some embodiments, however, wild-type IgSF domains are mutagenized (site specific, random, or combinations thereof) from wild-type IgSF genetic material and screened for altered binding according to the methods disclosed in the Examples. Methods or mutagenizing nucleic acids is known to those of skill in the art. In some embodiments, the affinity modified IgSF domains are synthesized de novo utilizing protein or nucleic acid sequences available at any number of publicly available databases and then subsequently screened. The National Center for Biotechnology Information provides such information and its website is publicly accessible via the internet as is the UniProtKB database as discussed previously.

In some embodiments, at least one non-affinity modified IgSF domain and/or one affinity modified IgSF domain present in the ectodomain of a transmembrane immunomodulatory protein provided herein specifically binds to at least one cell surface molecular species expressed on mammalian cells forming the immunological synapse (IS). In some embodiments, an ectodomain of a transmembrane immunomodulatory protein provided herein can comprise a plurality of non-affinity modified IgSF domains and/or affinity modified IgSF domains such as 1, 2, 3, 4, 5, or 6 non-affinity modified IgSF and/or affinity modified IgSF domains. One or more of these non-affinity modified IgSF domains and/or affinity modified IgSF domains can independently specifically bind to either one or both of the mammalian cells forming the IS.

Often, the cell surface molecular species to which the affinity modified IgSF domain of the ectodomain specifically binds will be the cognate binding partner of the wild type IgSF family member or wild type IgSF domain that has been affinity modified. In some embodiments, the cell surface molecular species is a mammalian IgSF member. In some embodiments, the cell surface molecular species is a human IgSF member. In some embodiments, the cell surface molecular species will be the cell surface cognate binding partners as indicated in Table 1. In some embodiments, the cell surface molecular species will be a viral protein, such as a poliovirus protein, on the cell surface of a mammalian cell such as a human cell.

In some embodiments, at least one non-affinity modified and/or affinity modified IgSF domain of the ectodomain of a transmembrane immunomodulatory protein provided herein binds to at least two or three cell surface molecular species present on mammalian cells forming the IS. The cell surface molecular species to which the non-affinity modified IgSF domains and/or the affinity modified IgSF domains of the ectodomain specifically bind to can exclusively be on one or the other of the two mammalian cells (i.e. in cis configuration) forming the IS or, alternatively, the cell surface molecular species can be present on both.

In some embodiments, the affinity modified IgSF domain specifically binds to at least two cell surface molecular species wherein one of the molecular species is present on one of the two mammalian cells forming the IS and the other molecular species is present on the second of the two mammalian cells forming the IS. In such embodiments, the cell surface molecular species is not necessarily present solely on one or the other of the two mammalian cells forming the IS (i.e., in a trans configuration) although in some embodiments it is. Thus, embodiments provided herein include those wherein each cell surface molecular species is exclusively on one or the other of the mammalian cells forming the IS (cis configuration) as well as those where the cell surface molecular species to which each affinity modified IgSF binds is present on both of the mammalian cells forming the IS (i.e., cis and trans configuration).

Those of skill will recognize that antigen presenting cells (APCs) and tumor cells form an immunological synapse with lymphocytes. Thus, in some embodiments at least one non-affinity modified IgSF domain and/or at least one affinity modified IgSF domain of the ectodomain of a transmembrane immunomodulatory protein specifically binds to only cell surface molecular species present on a cancer cell, wherein the cancer cell in conjunction with a lymphocyte forms the IS. In other embodiments, at least one non-affinity modified IgSF domain and/or at least one affinity modified IgSF domain of the ectodomain of a transmembrane immunomodulatory protein specifically binds to only cell surface molecular species present on a lymphocyte, wherein the lymphocyte in conjunction with an APC or tumor cell forms the IS. In some embodiments, the non-affinity modified IgSF domain and/or affinity modified IgSF domain bind to cell surface molecular species present on both the target cell (or APC) and the lymphocyte forming the IS.

Embodiments of the invention include those in which an ectodomain of a transmembrane immunomodulatory protein provided herein comprises at least one affinity modified IgSF domain with an amino acid sequence that differs from a wild-type or unmodified IgSF domain (e.g. a mammalian IgSF domain) such that the binding affinity (or avidity if in a multimeric or other relevant structure) of the affinity-modified IgSF domain, under specific binding conditions, to at least one of its cognate binding partners is either increased or decreased relative to the unaltered wild-type or unmodified IgSF domain control. In some embodiments, an affinity modified IgSF domain has a binding affinity for a cognate binding partner that differs from that of a wild-type or unmodified IgSF control sequence as determined by, for example, solid-phase ELISA immunoassays, flow cytometry or Biacore assays. In some embodiments, the affinity modified IgSF domain has an increased binding affinity for one or more cognate binding partners, relative to a wild-type or unmodified IgSF domain. In some embodiments, the affinity modified IgSF domain has a decreased binding affinity for one or more cognate binding partners, relative to a wild-type or unmodified IgSF domain. In some embodiments, the cognate binding partner can be a mammalian protein, such as a human protein or a murine protein.

Binding affinities for each of the cognate binding partners are independent; that is, in some embodiments, an affinity modified IgSF domain has an increased binding affinity for one, two or three different cognate binding partners, and a decreased binding affinity for one, two or three of different cognate binding partners, relative to a wild-type or unmodified polypeptide.

In some embodiments, the ectodomain of a transmembrane immunomodulatory protein provided herein comprises at least one affinity modified domain in which the binding affinity or avidity of the affinity modified IgSF domain is increased at least 10%, 20%, 30%, 40%, 50%, 100%, 200%, 300%, 400%, 500%, 1000%, 5000%, or 10,000% relative to the wild type or unmodified control IgSF domain. In some embodiments, the increase in binding affinity relative to the wild-type or unmodified IgSF domain is more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold.

In some embodiments, the ectodomain of a transmembrane immunomodulatory protein provided herein comprises at least one affinity modified domain in which the binding affinity or avidity of the affinity modified IgSF domain is decreased at least 10%, and up to 20%, 30%, 40%, 50%, 60%, 70%, 80% or up to 90% relative to the wild type or unmodified control IgSF domain. In some embodiments, the decrease in binding affinity relative to the wild-type or unmodified IgSF domain is more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold.

In some embodiments, the ectodomain of a transmembrane immunomodulatory protein provided herein comprises at least one affinity modified domain in which its specific binding affinity to a cognate binding partner can be at least 1×10⁻⁵ M, 1×10⁻⁶ M, 1×10⁻⁷ M, 1×10⁻⁸ M, 1×10⁻⁹ M, 1×10⁻¹⁰ M or 1×10⁻11 M, or 1×10⁻¹² M.

In some embodiments, the ectodomain of a transmembrane immunomodulatory protein provided comprises at least two IgSF domains in which at least one of the IgSF domain is affinity modified while in some embodiments both are affinity modified, and wherein at least one of the affinity modified IgSF domains has increased affinity (or avidity) to its cognate binding partner and at least one affinity modified IgSF domain has a decreased affinity (or avidity) to its cognate binding partner. Functionally, and irrespective of whether specific binding to its cognate binding partner is increased or decreased, the transmembrane immunomodulatory protein comprising the ectodomain acts to enhance or suppress immunological activity of engineered immune cells, such as lymphocytes or antigen presenting cells, relative to engineered immune cells expressing the wild-type, parental molecule under the appropriate assay controls. In some embodiments, a transmembrane immunomodulatory protein comprising an ectodomain that comprises at least two affinity modified IgSF domains is one in which at least one of the affinity modified IgSF domains agonizes an activating receptor and at least one affinity modified IgSF domain acts to antagonize an inhibitory receptor. In some embodiments, an enhancement of immunological activity can be an increase of at least 10%, 20%, 30%, 40%, 50%, 75%, 100%, 200%, 300%, 400%, or 500% greater than a non-zero control value such as in a cytotoxic activity assay, an assay for assessing cellular cytokines or a cell proliferation assay. In some embodiments, suppression of immunological activity can be a decrease of at least 10%, and up to 20%, 30%, 40%, 50%, 60%, 70%, 80%, or 90%.

The affinity modified IgSF domains of the transmembrane immunomodulatory proteins of the invention can in some embodiments specifically bind competitively to its counter-structure. In other embodiments the affinity modified IgSF domains of the present invention specifically bind non-competitively to its counter-structure.

In some embodiments, the ectodomain of a transmembrane immunomodulatory protein provided herein contains an IgSF domain that otherwise binds to multiple cell surface molecular species but is affinity modified such that it substantially no longer specifically binds to one of its cognate cell surface molecular species. Thus, in these embodiments the specific binding to one of its cognate cell surface molecular species is reduced to specific binding of no more than 90% of the wild type level, such as no more than 80%, 70%, 60%, 50%, 40%, 30%, 20% or less. In some embodiments, the specific binding to one of its cognate cell surface molecular species is reduced to specific binding of no more than 10% of the wild type level and often no more than 7%, 5%, 3%, 1%, or no detectable or statistically significant specific binding.

In some embodiments, a specific binding site on a mammalian IgSF domain is inactivated or substantially inactivated with respect to at least one of the cell surface molecular species. Thus, for example, if a wild type IgSF domain specifically binds to exactly two cell surface molecular species then in some embodiments it is affinity modified to specifically bind to exactly one cell surface molecular species. And, if a wild type IgSF domain specifically binds to exactly three cell surface molecular species then in some embodiments it is affinity modified to specifically bind to exactly two cell surface molecular species. The IgSF domain that is affinity modified to substantially no longer specifically bind to one of its cognate cell surface molecular species can be an IgSF domain that otherwise specifically binds competitively or non-competitively to its cell surface molecular species. An illustrative example concerns native CD80 (B7-1) which specifically binds counter-structures: CD28, PD-L1, and CTLA4. In some embodiments, CD80 can be IgSF affinity modified to increase or attenuate its specific binding to CD28 and/or PD-L1 but not to specifically bind to any physiologically significant extent to CTLA4. The IgSF domain that is affinity modified to substantially no longer specifically bind to one of its cell surface counter-structures can be an IgSF domain that otherwise specifically binds competitively or non-competitively to its counter-structure. Those of skill will appreciate that a wild type IgSF domain that competitively binds to two cognate binding partners can nonetheless be inactivated with respect to exactly one of them if, for example, their binding sites are not precisely coextensive but merely overlap such that specific binding of one inhibits binding of the other cognate binding partner and yet both competitive binding sites are distinct.

The non-affinity modified IgSF domains and/or affinity modified IgSF domains of the ectodomain of provided transmembrane immunomodulatory proteins provided can in some embodiments specifically bind competitively to its cognate cell surface molecular species. In other embodiments the non-affinity modified IgSF domains and/or affinity modified IgSF domains of the ectodomain of a transmembrane immunomodulatory protein provided herein specifically bind non-competitively to its cognate cell surface molecular species. Any number of the non-affinity modified IgSF domains and/or affinity modified IgSF domains present in the ectodomain of a transmembrane immunomodulatory protein provided herein can specifically bind competitively or non-competitively.

In some embodiments, the ectodomain of a transmembrane immunomodulatory protein provided herein comprises at least two non-affinity modified IgSF domains, or at least one non-affinity modified IgSF domain and at least one affinity modified IgSF domain, or at least two affinity modified IgSF domains wherein one IgSF domain specifically binds competitively and a second IgSF domain binds non-competitively to its cognate cell surface molecular species. More generally, the ectodomain of a transmembrane immunomodulatory protein provided herein can comprise 1, 2, 3, 4, 5, or 6 competitive or 1, 2, 3, 4, 5, or 6 non-competitive binding non-affinity modified IgSF and/or affinity modified IgSF domains or any combination thereof. Thus, the ectodomain of an immunomodulatory protein provided herein can have the number of non-competitive and competitive binding IgSF domains, respectively, of: 0 and 1, 0 and 2, 0 and 3, 0 and 4, 1 and 0, 1 and 1, 1 and 2, 1 and 3, 2 and 0, 2 and 1, 2 and 2, 2 and 3, 3 and 0, 3 and 1, 3 and 2, 3 and 3, 4 and 0, 4 and 1, and, 4 and 2.

In some embodiments in which the ectodomain contains a plurality of IgSF domain, the plurality of non-affinity modified and/or affinity modified IgSF domains of the ectodomain of the transmembrane immunomodulatory protein provided herein need not be covalently linked directly to one another. In some embodiments, an intervening span of one or more amino acid residues indirectly covalently bonds the non-affinity modified and/or affinity modified IgSF domains to each other. The linkage can be via the N-terminal to C-terminal residues.

In some embodiments, the linkage can be made via side chains of amino acid residues that are not located at the N-terminus or C-terminus of the non-affinity modified or affinity modified IgSF domain. Thus, linkages can be made via terminal or internal amino acid residues or combinations thereof.

The “peptide linkers” that link the non-affinity modified and/or affinity modified IgSF domains can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. In some embodiments, the linker is (in one-letter amino acid code): GGGGS (“4GS”) or multimers of the 4GS linker, such as repeats of 2, 3, 4, or 5 4GS linkers.

Exemplary Affinity Modified Domains and Ectodomains

In some embodiments, the ectodomain of a provided transmembrane immunomodulatory protein contains an affinity modified IgSF domain that has one or more amino acid substitutions in an IgSF domain of a wild-type or unmodified IgSF protein, such as set forth in Table 1 above. In some embodiments, the one or more amino acid substitutions are in the IgV domain or specific binding fragment thereof. In some embodiments, the one or more amino acid substitutions are in the IgC domain or specific binding fragment thereof. In some embodiments, one or more amino acid substitutions are in the IgV domain or a specific binding fragment thereof, and some of the one or more amino acid substitutions are in the IgC domain or a specific binding fragment thereof.

In some embodiments, the affinity modified IgSF domain of the ectodomain has up to 1, 2, 3, 4, 5, 6, 7, 8, 9, 10, 11, 12, 13, 14, 15, 16, 17, 18, 19, or 20 amino acid substitutions. The substitutions can be in the IgV domain or the IgC domain. In some embodiments, the affinity modified IgSF domain has up to 1, 2, 3, 4, 5, 6, 7, 8, 9, 10, 11, 12, 13, 14, 15, 16, 17, 18, 19, or 20 amino acid substitutions in the IgV domain or specific binding fragment thereof. In some embodiments, the affinity modified IgSF domain has up to 1, 2, 3, 4, 5, 6, 7, 8, 9, 10, 11, 12, 13, 14, 15, 16, 17, 18, 19, or 20 amino acid substitutions in the IgC domain or specific binding fragment thereof. In some embodiments, the affinity modified IgSF domain has at least about 85%, 86%, 86%, 88%, 89%, 90%, 91%, 92%, 93%, 94%, 95%, 96%, 97%, 98%, or 99% sequence identity with the wild-type or unmodified IgSF domain or specific binding fragment thereof, such as an IgSF domain contained in the IgSF protein set forth in any of SEQ ID NOS: 1-27.

In some embodiments, the transmembrane immunomodulatory protein contains an ectodomain that includes at least one affinity modified IgSF domain containing one or more amino acid substitutions in a wild-type or unmodified IgSF domain of a B7 IgSF family member. In some embodiments, the B7 IgSF family member is CD80, CD86 or ICOS Ligand (ICOSL). In some embodiments, the affinity modified IgSF domain of the ectodomain has at least about 85%, 86%, 86%, 88%, 89%, 90%, 91%, 92%, 93%, 94%, 95%, 96%, 97%, 98%, or 99% sequence identity with the wild-type or unmodified IgSF domain or specific binding fragment thereof of CD80, CD86 or ICOS Ligand (ICOSL), such as the IgSF domain (e.g. IgV or IgC) contained in the IgSF protein set forth in any of SEQ ID NOS: 1, 2 or 5. Exemplary affinity modified IgSF domains of CD80 are set forth in Table 2. Exemplary affinity modified IgSF domains of ICOSL are set forth in Table 3. Exemplary affinity modified IgSF domains of CD86 are set forth in Table 4.

TABLE 2
Exemplary variant CD80 polypeptides

| | ECD | IgV |
|---|---|---|
| | SEQ ID | SEQ ID |
| Mutation(s) | NO | NO |
| Wild-type | 28 | 152 |
| L70Q/A91G | 55 | 153 |
| L70Q/A91G/T130A | 56 | |
| L70Q/A91G/I118A/T120S/T130A | 57 | |
| V4M/L70Q/A91G/T120S/T130A | 58 | 154 |
| L70Q/A91G/T120S/T130A | 59 | |
| V20L/L70Q/A91S/T120S/T130A | 60 | 155 |
| S44P/L70Q/A91G/T130A | 61 | 156 |
| L70Q/A91G/E117G/T120S/T130A | 62 | |
| A91G/T120S/T130A | 63 | 157 |
| L70R/A91G/T120S/T130A | 64 | 158 |
| L70Q/E81A/A91G/T120S/I127T/T130A | 65 | 159 |
| L70Q/Y87N/A91G/T130A | 66 | 160 |
| T28S/L70Q/A91G/E95K/T120S/T130A | 67 | 161 |
| N63S/L70Q/A91G/T120S/T130A | 68 | 162 |
| K36E/I67T/L70Q/A91G/T120S/T130A/N152T | 69 | 163 |
| E52G/L70Q/A91G/T120S/T130A | 70 | 164 |
| K37E/F59S/L70Q/A91G/T120S/T130A | 71 | 165 |
| A91G/S103P | 72 | |
| K89E/T130A | 73 | 166 |
| A91G | 74 | |
| D60V/A91G/T120S/T130A | 75 | 167 |
| K54M/A91G/T120S | 76 | 168 |
| M38T/L70Q/E77G/A91G/T120S/T130A/N152T | 77 | 169 |
| R29H/E52G/L70R/E88G/A91G/T130A | 78 | 170 |
| Y31H/T41G/L70Q/A91G/T120S/T130A | 79 | 171 |
| V68A/110A | 80 | 172 |
| S66H/D90G/T110A/F116L | 81 | 173 |
| R29H/E52G/T120S/T130A | 82 | 174 |
| A91G/L102S | 83 | |
| I67T/L70Q/A91G/T120S | 84 | 175 |
| L70Q/A91G/T110A/T120S/T130A | 85 | |
| M38V/T41D/M43I/W50G/D76G/V83A/K89E/ | 86 | 176 |
| T120S/T130A | | |
| V22A/L70Q/S121P | 87 | 177 |
| A12V/S15F/Y31H/T41G/T130A/P137L/N152T | 88 | 178 |
| I67F/L70R/E88G/A91G/T120S/T130A | 89 | 179 |
| E24G/L25P/L70Q/T120S | 90 | 180 |
| A91G/F92L/F108L/T120S | 91 | 181 |
| R29D/Y31L/Q33H/K36G/M38I/T41A/M43R/ | 92 | 182 |
| M47T/E81V/L85R/K89N/A91T/F92P/K93V/ | | |
| R94L/I118T/N149S | | |
| R29D/Y31L/Q33H/K36G/M38I/T41A/M43R/ | 93 | |
| M47T/E81V/L85R/K89N/A91T/F92P/K93V/ | | |
| R94L/N144S/N149S | | |
| R29D/Y31L/Q33H/K36G/M38I/T41A/M42T/ | 94 | 183 |
| M43R/M47T/E81V/L85R/K89N/A91T/F92P/ | | |
| K93V/R94L/L148S/N149S | | |
| E24G/R29D/Y31L/Q33H/K36G/M38I/T41A/ | 95 | 184 |
| M43R/M47T/F59L/E81V/L85R/K89N/A91T/ | | |
| F92P/K93V/R94L/H96R/N149S/C182S | | |
| R29D/Y31L/Q33H/K36G/M38I/T41A/M43R/ | 96 | |
| M47T/E81V/L85R/K89N/A91T/F92P/ | | |
| K93V/R94L/N149S | | |
| R29V/M43Q/E81R/L85I/K89R/D90L/ | 97 | 185 |
| A91E/F92N/K93Q/R94G | | |
| T41I/A91G | 98 | 186 |
| K89R/D90K/A91G/F92Y/K93R/N122S/N177S | 99 | 187 |
| K89R/D90K/A91G/F92Y/K93R | 100 | |
| K36G/K37Q/M38I/F59L/E81V/L85R/ | 101 | 188 |
| K89N/A91T/F92P/K93V/R94L/E99G/ | | |
| T130A/N149S | | |
| E88D/K89R/D90K/A91G/F92Y/K93R | 102 | 189 |
| K36G/K37Q/M38I/L40M | 103 | 190 |
| K36G | 104 | 191 |
| R29H/Y31H/T41G/Y87N/E88G/K89E/ | 105 | 192 |
| D90N/A91G/P109S | | |
| A12T/H18L/N43V/F59L/E77K/P109S/I118T | 106 | 193 |
| R29V/Y31F/K36G/M38L/N43Q/E81R/V83I/ | 107 | 194 |
| L85I/K89R/D90L/A91E/F92N/K93Q/R94G | | |
| V68M/L70P/L72P/K86E | 108 | 195 |

TABLE 3
Exemplary variant ICOSL polypeptides

| | ECD | IgV |
|---|---|---|
| | SEQ ID | SEQ ID |
| Mutation(s) | NO | NO |
| Wild-type | 32 | 196 |
| N52S | 109 | 197 |
| N52H | 110 | 198 |
| N52D | 111 | 199 |
| N52Y/N57Y/F138L/L203P | 112 | 200 |
| N52H/N57Y/Q100P | 113 | 201 |
| N52S/Y146C/Y152C | 114 | |
| N52H/C198R | 115 | |
| N52H/C140D/T225A | 116 | |
| N52H/C198R/T225A | 117 | |
| N52H/K92R | 118 | 202 |
| N52H/S99G | 119 | 203 |
| N52Y | 120 | 204 |
| N57Y | 121 | 205 |
| N57Y/Q100P | 122 | 206 |
| N52S/S130G/Y152C | 123 | |
| N52S/Y152C | 124 | |
| N52S/C198R | 125 | |
| N52Y/N57Y/Y152C | 126 | |
| N52Y/N57Y/129P/C198R | 127 | |
| N52H/L161P/C198R | 128 | |
| N52S/T113E | 129 | |
| S54A | 130 | 207 |
| N52D/S54P | 131 | 208 |
| N52K/L208P | 132 | 209 |
| N52S/Y152H | 133 | |
| N52D/V151A | 134 | |
| N52H/I143T | 135 | |
| N52S/L80P | 136 | 210 |
| F120S/Y152H/N201S | 137 | |
| N52S/R75Q/L203P | 138 | 211 |
| N52S/D158G | 139 | |
| N52D/Q133H | 140 | |
| N52S/N57Y/H94D/L96F/L98F/Q100R | 141 | 212 |
| N52S/N57Y/H94D/L96F/L98F/Q100R/G103E/F120S | 142 | 213 |
| N52S/G103E | 239 | 240 |

TABLE 4
Exemplary variant CD86 polypeptides

| | ECD | IgC |
|---|---|---|
| | SEQ ID | SEQ ID |
| Mutation(s) | NO | NO |
| Wild-type | 29 | 220 |
| Q35H/H90L/Q102H | 148 | 221 |
| Q35H | 149 | 222 |
| H90L | 150 | 223 |
| Q102H | 151 | 224 |

In some embodiments, the transmembrane immunomodulatory protein contains an ectodomain that includes at least one affinity modified IgSF domain containing one or more amino acid substitutions in a wild-type or unmodified IgSF domain of a poliovirus receptor IgSF family member. In some embodiments, the poliovirus IgSF family member is CD155 (PVR) or CD122 (PRR-2). In some embodiments, the affinity modified IgSF domain of the ectodomain has at least about 85%, 86%, 86%, 88%, 89%, 90%, 91%, 92%, 93%, 94%, 95%, 96%, 97%, 98%, or 99% sequence identity with the wild-type or unmodified IgSF domain or specific binding fragment thereof of CD155 or CD112, such as an IgSF domain (e.g. IgV or IgC) contained in the IgSF protein set forth in any of SEQ ID NO:20 or 21. Exemplary affinity modified IgSF domains of CD155 are set forth in Table 5. Exemplary affinity modified IgSF domains of CD112 are set forth in Table 6.

TABLE 5
Exemplary variant CD155 polypeptides

| | ECD | IgV |
|---|---|---|
| | SEQ ID | SEQ ID |
| Mutation(s) | NO | NO |
| Wild-type | 47 | 241 |
| P18S, P64S, F91S | 242 | 264 |
| P18S, F91S, L104P | 243 | 265 |
| L44P | 244 | 266 |
| A56V | 245 | 267 |
| P18L, L79V, F91S | 246 | 268 |
| P18S, F91S | 247 | 269 |
| P18T, F91S | 248 | 270 |
| P18T, S42P, F91S | 249 | 271 |
| G7E, P18T, Y30C, F91S | 250 | 272 |
| P18T, F91S, G111D | 251 | 273 |
| P18S, F91P | 252 | 274 |
| P18T, F91S, F108L | 253 | 275 |
| P18S, F91S | 254 | 276 |
| P18T, T45A, F91S | 255 | 277 |
| P18T, F91S, R94H | 256 | 278 |
| P18S, Y30C, F91S | 257 | 279 |
| A81V, L83P | 258 | 280 |
| L88P | 259 | 281 |
| R94H | 260 | 282 |
| A13E, P18S, A56V, F91S | 261 | 283 |
| P18T, F91S, V115A | 262 | 284 |
| P18T, Q60K | 263 | 285 |

TABLE 6
Exemplary variant CD112 polypeptides

| | ECD | IgV |
|---|---|---|
| | SEQ ID | SEQ ID |
| Mutation(s) | NO | NO |
| Wild-type | 48 | 286 |
| Y33H, A112V, G117D | 287 | 334 |
| V19A, Y33H, S64G, S80G, G98S, N106Y, A112V | 288 | 335 |
| L32P, A112V | 289 | 336 |
| A95V, A112I | 290 | 337 |
| P28S, A112V | 291 | 338 |
| P27A, T38N, V101A, A112V | 292 | 339 |
| S118F | 293 | 340 |
| R12W, H48Y, F54S, S118F | 294 | 341 |
| R12W, Q79R, S118F | 295 | 342 |
| T113S, S118Y | 296 | 343 |
| S118Y | 297 | 344 |
| N106I, S118Y | 298 | 345 |
| N106I, S118F | 299 | 346 |
| A95T, L96P, S118Y | 300 | 347 |
| Y33H, P67S, N106Y, A112V | 301 | 348 |
| N106Y, A112V | 302 | 349 |
| T18S, Y33H, A112V | 303 | 350 |
| P9S, Y33H, N47S, A112V | 304 | 351 |
| P42S, P67H, A112V | 305 | 352 |
| P27L, L32P, P42S, A112V | 306 | 353 |
| G98D, A112V | 307 | 354 |
| Y33H, S35P, N106Y, A112V | 308 | 355 |
| L32P, P42S, T100A, A112V | 309 | 356 |
| P27S, P45S, N106I, A112V | 310 | 357 |
| Y33H, N47K, A112V | 311 | 358 |
| Y33H, N106Y, A112V | 312 | 359 |
| K78R, D84G, A112V, F114S | 313 | 360 |
| Y33H, N47K, F54L, A112V | 314 | 361 |
| Y33H, A112V | 315 | 362 |
| A95V, A112V | 316 | 363 |
| R12W, A112V | 317 | 364 |
| R12W, P27S, A112V | 318 | 365 |
| Y33H, V51M, A112V | 319 | 366 |
| Y33H, A112V, S118T | 320 | 367 |
| Y33H, V101A, A112V, P115S | 321 | 368 |
| H24R, T38N, D43G, A112V | 322 | 369 |
| A112V | 323 | 370 |
| P27A, A112V | 324 | 371 |
| A112V, S118T | 325 | 372 |
| R12W, A112V, M122I | 326 | 373 |
| Q83K, N106Y, A112V | 327 | 374 |
| R12W, P27S, A112V, S118T | 328 | 375 |
| P28S,Y33H, A112V | 329 | 376 |
| P27S, Q90R, A112V | 330 | 377 |
| L15V, P27A, A112V, S118T | 331 | 378 |
| Y33H, N106Y, T108I, A112V | 332 | 379 |
| Y33H, P56L, V75M, V101M, A112V | 333 | 380 |

In some embodiments, the transmembrane immunomodulatory protein contains an ectodomain that includes an affinity modified IgSF domain containing one or more amino acid substitutions in a wild-type or unmodified IgSF domain of an NkP30 family member. In some embodiments, the affinity modified IgSF domain has at least about 85%, 86%, 86%, 88%, 89%, 90%, 91%, 92%, 93%, 94%, 95%, 96%, 97%, 98%, or 99% sequence identity with the wild-type or unmodified IgSF domain or specific binding fragment thereof of an NkP30 family member, such as the IgSF domain (e.g. IgC) contained in the IgSF protein set forth in SEQ ID NO: 27. Table 7 provides exemplary affinity modified NkP30 IgSF domains.

TABLE 7
Exemplary variant NKp30 polypeptides

| | ECD | IgV |
|---|---|---|
| | SEQ ID | SEQ ID |
| Mutation(s) | NO | NO |
| Wild-type | 54 | 214 |
| L30V/A60V/S64P/S86G | 143 | 215 |
| L30V | 144 | 216 |
| A60V | 145 | 217 |
| S64P | 146 | 218 |
| S86G | 147 | 219 |

B. Transmembrane Domain

The transmembrane immunomodulatory proteins provided herein further contain a transmembrane domain linked to the ectodomain. In some embodiments, the transmembrane domain results in an encoded protein for cell surface expression on a cell. In some embodiments, the transmembrane domain is linked directly to the ectodomain. In some embodiments, the transmembrane domain is linked indirectly to the ectodomain via one or more linkers or spacers. In some embodiments, the transmembrane domain contains predominantly hydrophobic amino acid residues, such as leucine and valine.

In some embodiments, a full length transmembrane anchor domain can be used to ensure that the TIPs will be expressed on the surface of the engineered cell, such as engineered T cell. Conveniently, this could be from a particular native protein that is being affinity modified (e.g. CD80 or ICOSL or other native IgSF protein), and simply fused to the sequence of the first membrane proximal domain in a similar fashion as the native IgSF protein (e.g. CD80 or ICOSL). In some embodiments, the transmembrane immunomodulatory protein comprises a transmembrane domain of the corresponding wild-type or unmodified IgSF member, such as a transmembrane domain contained in the sequence of amino acids set forth in any of SEQ ID NOs:1-27 (see Table 1).

In some embodiments, the transmembrane domain is a non-native transmembrane domain that is not the transmembrane domain of the wild-type IgSF member. In some embodiments, the transmembrane domain is derived from a transmembrane domain from another non-IgSF family member polypeptide that is a membrane-bound or is a transmembrane protein. In some embodiments, a transmembrane anchor domain from another protein on T cells can be used. In some embodiments, the transmembrane domain is derived from CD8. In some embodiments, the transmembrane domain can further contain an extracellular portion of CD8 that serves as a spacer domain. An exemplary CD8 derived transmembrane domain is set forth in SEQ ID NO: 386 or a portion thereof containing the CD8 transmembrane domain. In some embodiments, the transmembrane domain is a synthetic transmembrane domain.

C. Endodomain

In some embodiments, the transmembrane immunomodulatory protein further contains an endodomain, such as a cytoplasmic signaling domain, linked to the transmembrane domain. In some embodiments, the cytoplasmic signaling domain induces cell signaling. In some embodiments, the endodomain of the transmembrane immunomodulatory protein comprises the cytoplasmic domain of the corresponding wild-type or unmodified polypeptide, such as a cytoplasmic domain contained in the sequence of amino acids set forth in any of SEQ ID NOS:1-27 (see Table 1).

In some embodiments, provided are CAR-related transmembrane immunomodulatory proteins in which the endodomain of a transmembrane immunomodulatory protein comprises a cytoplasmic signaling domain that comprises at least one ITAM (immunoreceptor tyrosine-based activation motif)-containing signaling domain. ITAM is a conserved motif found in a number of protein signaling domains involved in signal transduction of immune cells, including in the CD3-zeta chain (“CD3-z”) involved in T-cell receptor signal transduction. In some embodiments, the endodomain comprises at CD3-zeta signaling domain. In some embodiments, the CD3-zeta signaling domain comprises the sequence of amino acids set forth in SEQ ID NO: 387 or a sequence of amino acids that exhibits at least 85%, 86%, 87%, 88%, 89%, 90%, 91%, 92%, 93%, 94%, 95%, 96%, 97%, 98% or 99% to SEQ ID NO:387 and retains the activity of T cell signaling. In some embodiments, the endodomain of a CAR-related transmembrane immunomodulatory protein can further comprise a costimulatory signaling domain to further modulate immunomodulatory responses of the T-cell. In some embodiments, the costimulatory signaling domain is CD28, ICOS, 41BB or OX40. In some embodiments, the provided CAR-related transmembrane immunomodulatory proteins have features of CARs to stimulate T cell signaling upon binding of an affinity modified IgSF domain to a cognate binding partner or counter structure. In some embodiments, upon specific binding by the affinity-modified IgSF domain to its counter structure can lead to changes in the immunological activity of the T-cell activity as reflected by changes in cytotoxicity, proliferation or cytokine production.

In some embodiments, a CAR-related transmembrane immunomodulatory protein comprises an antigen binding region that is engineered to specifically bind to a desired counter-structure. In some embodiments, an affinity modified IgSF domain specifically binds its native counter-structure. In some embodiments the counter-structure is an IgSF family member. In some embodiments, provided is a CAR-related transmembrane immunomodulatory protein that specifically binds to a tumor specific IgSF counter-structure. In some embodiments, the antigen binding region (ectodomain) is an affinity modified IgSF domain NKp30. In some embodiments, the affinity modified IgSF domain specifically binds the tumor specific antigen NKp30 ligand B7-H6 (see, Levin et al., The Journal of Immunology, 2009, 182, 134.20). In examples of such embodiments, the endodomain comprises at least one ITAM (immunoreceptor tyrosine-based activation motif) containing signaling domain, such as a CD3-zeta signaling domain. In some embodiments, the endodomain can further comprises at least one of: a CD28 costimulatory domain, an OX40 signaling domain, and a 41BB signaling domain.

In some embodiments, the transmembrane immunomodulatory protein does not contain an endodomain capable of mediating cytoplasmic signaling. In some embodiments, the transmembrane immunomodulatory protein lacks the signal transduction mechanism of the wild-type or unmodified polypeptide and therefore does not itself induce cell signaling. In some embodiments, the transmembrane immunomodulatory protein lacks an intracellular (cytoplasmic) domain or a portion of the intracellular domain of the corresponding wild-type or unmodified polypeptide, such as a cytoplasmic signaling domain contained in the sequence of amino acids set forth in any of SEQ ID NOS:1-27 (see Table 1). In some embodiments, the transmembrane immunomodulatory protein does not contain an ITIM (immunoreceptor tyrosine-based inhibition motif), such as contained in certain inhibitory receptors, including inhibitory receptors of the IgSF family (e.g. PD-1 or TIGIT). Thus, in some embodiments, the transmembrane immunomodulatory protein only contains the ectodomain and the transmembrane domain, such as any as described.

D. Nucleic Acid Molecules and Vectors

Provided herein are isolated or recombinant nucleic acids collectively referred to as “nucleic acids” which encode any of the various provided embodiments of the transmembrane immunomodulatory polypeptides of the invention. Nucleic acids provided herein, including all described below, are useful in recombinant expression of the transmembrane immunomodulatory proteins, including for engineering cells. The nucleic acids provided herein can be in the form of RNA or in the form of DNA, and include mRNA, cRNA, recombinant or synthetic RNA and DNA, and cDNA. The nucleic acids of the invention are typically DNA molecules, and usually double-stranded DNA molecules. However, single-stranded DNA, single-stranded RNA, double-stranded RNA, and hybrid DNA/RNA nucleic acids or combinations thereof comprising any of the nucleotide sequences of the invention also are provided.

Also provided herein are expression vectors useful in engineering cells to express the transmembrane immunomodulatory proteins of the present invention. The immunomodulatory polypeptides provided herein can be introduced into cells using recombinant DNA techniques. To do so, a recombinant DNA molecule encoding a transmembrane immunomodulatory polypeptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidite method. Also, a combination of these techniques could be used. In some instances, a recombinant or synthetic nucleic acid may be generated through polymerase chain reaction (PCR).

In some embodiments, a full length DNA insert can be generated comprising an optional endodomain (i.e., cytoplasmic domain), a transmembrane anchor domain, an optional spacer domain, an optional epitope tag, and finally one or more extracellular affinity modified IgSF domains. This DNA insert can be cloned into an appropriate T cell transduction/transfection vector as is known to those of skill in the art. Also provided are vectors containing the nucleic acid molecules.

In some embodiments, the expression vectors are capable of expressing the transmembrane immunomodulatory proteins in an appropriate cell under conditions suited to expression of the protein. In some aspects, an expression vector comprises the DNA molecule that codes for the transmembrane immunomodulatory protein operatively linked to appropriate expression control sequences. Methods of affecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation. In some embodiments, a nucleic acid of the invention further comprises nucleotide sequence that encodes a secretory or signal peptide operably linked to the nucleic acid encoding the transmembrane immunomodulatory protein.

In some embodiments, the resulting expression vector having the DNA molecule thereon is used to transform, such as transduce, an appropriate cell. The introduction can be performed using methods well known in the art. Exemplary methods include those for transfer of nucleic acids encoding the receptors, including via viral, e.g., retroviral or lentiviral, transduction, transposons, and electroporation. In some embodiments, the expression vector is a viral vector. In some embodiments, the nucleic acid is transferred into cells by lentiviral or retroviral transduction methods.

E. Exemplary Transmembrane Immunomodulatory Proteins and Encoding Nucleic Acid Molecules

Provided herein is a transmembrane immunomodulatory protein in accord with the above description that comprises a sequence of amino acids that exhibits at least 85%, 86%, 87%, 88%, 89%, 90%, 91%, 92%, 93%, 94%, 95%, 96%, 97%, 98% or 99% sequence identity to any of SEQ ID NOS: 393-419 and contains an ectodomain comprising at least one affinity-modified IgSF domain as described and a transmembrane domain. In some embodiments, the transmembrane immunomodulatory protein can further comprise a cytoplasmic domain as described. In some embodiments, the transmembrane immunomodulatory protein can further contain a signal peptide. In some embodiments, the signal peptide is the native signal peptide of the corresponding wild-type IgSF member (see e.g. Table 1).

Also provided herein is a nucleic acid molecule encoding a transmembrane immunomodulatory protein comprising a nucleotide sequence that encodes a sequence of amino acids that exhibits at least 85%, 86%, 87%, 88%, 89%, 90%, 91%, 92%, 93%, 94%, 95%, 96%, 97%, 98% or 99% sequence identity to any of SEQ ID NOS: 393-419 and contains an ectodomain comprising at least one affinity-modified IgSF domain as described, a transmembrane domain and, optionally, a cytoplasmic domain. In some embodiments, the nucleic acid molecule can further comprise a sequence of nucleotides encoding a signal peptide. In some embodiments, the signal peptide is the native signal peptide of the corresponding wild-type IgSF member (see e.g. Table 1).

Exemplary of a transmembrane immunomodulatory protein is a CD80 TIP comprising i) the sequence of amino acids set forth in SEQ ID NO:381 or ii) a sequence of amino acids that exhibits at least 85%, 86%, 87%, 88%, 89%, 90%, 91%, 92%, 93%, 94%, 95%, 96%, 97%, 98% or 99% sequence identity to SEQ ID NO:381 and that comprises the affinity-modified domain of SEQ ID NO:381. Also provided is i) a sequence of nucleotides set forth in SEQ ID NO:382, ii) a sequence that exhibits at least 85%, 86%, 87%, 88%, 89%, 90%, 91%, 92%, 93%, 94%, 95%, 96%, 97%, 98% or 99% sequence identity to SEQ ID NO: 381 and that encodes a TIP that comprises the affinity-modified domain of SEQ ID NO:381, or iii) a sequence of i) or ii) having degenerate codons.

Exemplary of a transmembrane immunomodulatory protein is a ICOSL TIP comprising i) the sequence of amino acids set forth in SEQ ID NO:383 or ii) a sequence of amino acids that exhibits at least 85%, 86%, 87%, 88%, 89%, 90%, 91%, 92%, 93%, 94%, 95%, 96%, 97%, 98% or 99% sequence identity to SEQ ID NO:381 and that comprises the affinity-modified domain of SEQ ID NO:383. Also provided is i) a sequence of nucleotides set forth in SEQ ID NO:384, ii) a sequence that exhibits at least 85%, 86%, 87%, 88%, 89%, 90%, 91%, 92%, 93%, 94%, 95%, 96%, 97%, 98% or 99% sequence identity to SEQ ID NO: 384 and that encodes a TIP that comprises the affinity-modified domain of SEQ ID NO:383, or iii) a sequence of i) or ii) having degenerate codons.

### III. ENGINEERED CELLS

Provided herein are engineered cells expressing on their surface any of the provided transmembrane immunomodulatory polypeptide. In some embodiments, the transmembrane immunomodulatory protein is expressed on a lymphocyte such as a tumor infiltrating lymphocyte (TIL), T-cell or NK cell, or on a myeloid cell. In some embodiments, the engineered cells are antigen presenting cells (APCs). In some embodiments, the engineered cells are engineered mammalian T cells or engineered mammalian antigen presenting cells (APCs). In some embodiments, the engineered T-cells or APCs are human or murine cells.

In some embodiments, engineered T-cells include, but are not limited to, T helper cell, cytotoxic T-cell (alternatively, cytotoxic T lymphocyte or CTL), natural killer T-cell, regulatory T-cell, memory T-cell, or gamma delta T-cell. In some embodiments, the engineered T cells are CD4+ or CD8+. In addition to the signal of the MHC, engineered T-cells also require a co-stimulatory signal which in some embodiments is provided by the TIP as discussed previously.

In some embodiments, the engineered APCs include, for example, MHC II expressing APCs such as macrophages, B cells, and dendritic cells, as well as artificial APCs (aAPCs) including both cellular and acellular (e.g., biodegradable polymeric microparticles) aAPCs. Artificial APCs (aAPCs) are synthetic versions of APCs that can act in a similar manner to APCs in that they present antigens to T-cells as well as activate them. Antigen presentation is performed by the MHC (Class I or Class II). In some aspects, in engineered APCs such as aAPCs, the antigen that is loaded onto the MHC is, in some embodiments, a tumor specific antigen or a tumor associated antigen. The antigen loaded onto the MHC is recognized by a T-cell receptor (TCR) of a T cell, which, in some cases, can express one or more cognate binding partners or other molecule recognized by the affinity modified domain of the transmembrane immunomodulatory polypeptides provided herein. Materials which can be used to engineer an aAPC include: poly (glycolic acid), poly(lactic-co-glycolic acid), iron-oxide, liposomes, lipid bilayers, sepharose, and polystyrene.

In some embodiments, a transmembrane immunomodulatory protein provided herein is co-expressed or engineered into a cell that expresses an antigen-binding receptor, such as a recombinant receptor, such as a chimeric antigen receptor (CAR) or T cell receptor (TCR). In some embodiments, the engineered cell, such as an engineered T cell, recognizes a desired antigen associated with cancer, inflammatory and autoimmune disorders, or a viral infection. In specific embodiments, the antigen-binding receptor contains an antigen-binding moiety that specifically binds a tumor specific antigen or a tumor associated antigen. In some embodiments, the engineered T-cell is a CAR (chimeric antigen receptor) T-cell that contains an antigen-binding domain (e.g. scFv) that specifically binds to an antigen, such as a tumor specific antigen or tumor associated antigen. In another embodiment, the engineered T-cell possesses a TCR, including a recombinant or engineered TCR. In some embodiments, the TCR can be a native TCR. Those of skill in the art will recognize that generally native mammalian T-cell receptors comprise an alpha and a beta chain (or a gamma and a delta chain) involved in antigen specific recognition and binding. In some embodiments, the TCR is an engineered TCR that is modified. In some embodiments, the TCR of an engineered T-cell specifically binds to a tumor associated or tumor specific antigen presented by an APC. Thus, in some embodiments, the TIP protein is expressed in an engineered T-cell receptor cell or and engineered chimeric antigen receptor cell. In such embodiments, the engineered cell co-expresses the TIP and the CAR or TCR.

In some embodiments, transmembrane immunomodulatory polypeptides can be incorporated into engineered cells, such as engineered T cells or engineered APCs, by a variety of strategies such as those employed for recombinant host cells. A variety of methods to introduce a DNA construct into primary T cells are known in the art. In some embodiments, viral transduction or plasmid electroporation are employed. In typical embodiments, the nucleic acid molecule encoding the transmembrane immunomodulatory protein, or the expression vector, comprises a signal peptide that localizes the expressed transmembrane immunomodulatory proteins to the cellular membrane. In some embodiments, a nucleic acid encoding a transmembrane immunomodulatory proteins of the invention is sub-cloned into a viral vector, such as a retroviral vector, which allows expression in the host mammalian cell. The expression vector can be introduced into a mammalian host cell and, under host cell culture conditions, the TIP is expressed.

In an exemplary example, primary T cells can be purified ex vivo (CD4 cells or CD8 cells or both) and stimulated with an activation protocol consisting of various TCR/CD28 agonists, such as anti-CD3/anti-CD28 coated beads. After a 2 or 3 day activation process, the DNA vector containing a TIP of the present invention can be stably introduced into the primary T cells through art standard lentiviral or retroviral transduction protocols or plasmid electroporation strategies. Cells can be monitored for TIP expression by, for example, flow cytometry using anti-epitope tag or antibodies that cross-react with native parental molecule and affinity modified variant. T cells that express the TIP can be enriched through sorting with anti-epitope tag antibodies or enriched for high or low expression depending on the application.

Upon TIP expression the engineered T cell can be assayed for improved function by a variety of means. The engineered CAR or TCR co-expression can be validated to show that this part of the engineered T cell was not significantly impacted by the expression of the TIP construct. Once validated, standard in vitro cytotoxicity, proliferation, or cytokine assays can be used to assess the function of the engineered cells. Exemplary standard endpoints are percent lysis of the tumor line, proliferation of the engineered T-cell, or IFN-gamma protein expression in culture supernatants. An engineered construct which results in statistically significant increased lysis of tumor line, increased proliferation of the engineered T-cell, or increased IFN-gamma expression over the control construct can be selected for. Additionally, non-engineered cells, such as native primary or endogenous T-cells, could also be incorporated into the same in vitro assay to measure the ability of the TIP construct expressed on the engineered cells, such as engineered T-cells, to modulate activity, including, in some cases, to activate and generate effector function in bystander, native T-cells. Increased expression of activation markers such as CD69, CD44, or CD62L could be monitored on endogenous T cells, and increased proliferation and/or cytokine production could indicate desired activity of the TIP expressed on the engineered T cells.

In some embodiments, the similar assays can be used to compare the function of engineered T cells containing the CAR or TCR alone to those containing the CAR or TCR and a TIP construct. Typically, these in vitro assays are performed by plating various ratios of the engineered T cell and a “tumor” cell line containing the cognate CAR or TCR antigen together in culture. Standard endpoints are percent lysis of the tumor line, proliferation of the engineered T cell, or IFN-gamma production in culture supernatatants. An engineered TIP construct which resulted in statistically significant increased lysis of tumor line, increased proliferation of the engineered T cell, or increased IFN-gamma production over the same TCR or CAR construct alone can be selected for. Engineered human T cells can be analyzed in immunocompromised mice, like the NSG strain, which lacks mouse T, NK and B cells. Engineered human T cells in which the CAR or TCR binds a target counter-structure on the xenograft and is co-expressed with the TIP affinity modified IgSF domain can be adoptively transferred in vivo at different cell numbers and ratios compared to the xenograft. For example, engraftment of CD19+ leukemia tumor lines containing a luciferase/GFP vector can be monitored through bioluminescence or ex vivo by flow cytometry. In a common embodiment, the xenograft is introduced into the murine model, followed by the engineered T cells several days later. Engineered T cells containing the TIP can be assayed for increased survival, tumor clearance, or expanded engineered T cells numbers relative to engineered T cells containing the CAR or TCR alone. As in the in vitro assay, endogenous, native (i.e., non-engineered) human T cells could be co-adoptively transferred to look for successful epitope spreading in that population, resulting in better survival or tumor clearance.

Exemplary Functional Activities and Features

In some aspects, TIP-engineered cells, such as engineered lymphocytes (e.g. tumor infiltrating lymphocytes, T cells or NK cells) or myeloid cells (e.g. antigen presenting cells), exhibit one or more desirable features or activities.

In some embodiments, the affinity-modified IgSF domain, localized on the ectodomain of the TIP, specifically binds to at least one counter-structure expressed on a mammalian cell. In some embodiments, the mammalian cell is an autologous or allogeneic mouse, rat, cynomolgus monkey, or human cell. In some aspects, the mammalian cell can include such embodiments as an antigen presenting cell (APC), a tumor cell, or a T-cell. In some embodiments, the tumor cell is a mouse, rat, cynomolgus monkey, or human tumor cell. A TIP can comprise one or multiple (e.g., 2, 3, or 4) affinity modified IgSF domains. Thus, in some embodiments the TIP binds to no more than one counter-structure on the mammalian cell. In some embodiments, an affinity modified IgSF domain of a TIP specifically binds to no more than one counter-structure on the mammalian cell. Alternatively, in some embodiments, a TIP specifically binds to at least two, three or four, or exactly two, three, or four, counter-structures expressed on a mammalian cell. In some embodiments, the TIP specifically binds to no more than one cell surface counter-structure. Specific binding of the TIP affinity-modified IgSF domain to the counter-structure on a mammalian cell modulates immunological activity of the mammalian cell. Specific binding by and between an affinity modified IgSF domain and a mammalian cell counter-structure can be specific binding in cis arrangement (i.e., specific binding on the same cell) or in trans arrangement (i.e., specific binding on different cells) or in both cis and trans arrangement. Immunological activity of the cell can be increased as evidenced by increased, e.g., cell survival, cell proliferation, cytokine production, or T-cell cytotoxicity. In alternative embodiments, the immunological activity of the cell is attenuated as evidenced by a decrease of cell survival, cell proliferation, cytokine production, or T-cell cytotoxicity.

In some embodiments, at least one affinity modified IgSF domain present in a transmembrane immunomodulatory protein specifically binds to at least one cell surface counter-structure expressed on a mammalian cell and in which modulation of immunological activity is desired. In some embodiments, the counter-structure to which the affinity modified IgSF domain specifically binds is the native counter-structure of the wild type IgSF family member or wild type IgSF domain that has been affinity modified. In some embodiments, the specific binding increases and/or attenuates activity a mammalian cell expressing a cognate binding partner to which the affinity modified IgSF domain exhibits improved binding. Thus, by increasing specific binding affinity, the provided transmembrane immunomodulatory proteins can either increase or attenuate immunological activity of a mammalian cell. In some embodiments, the specific binding modulates, such as increases, immunological activity of the engineered cell with the transmembrane immunomodulatory protein.

In some embodiments, the counter-structure expressed on the mammalian cell is a mammalian IgSF member. The mammalian cell is, in some embodiments, an antigen presenting cell (APC), a lymphocyte, or a tumor cell. In some embodiments, the lymphocyte is a tumor infiltrating lymphocyte (TIL), an engineered or native T-cell, or an engineered or native NK cell. In some embodiments, the counter-structure of the affinity modified IgSF domain is a native human IgSF member. In some embodiments, the counter-structure is a “cell surface cognate binding partner” as indicated in Table 1.

In some embodiments, a TIP comprising an affinity modified IgSF when expressed on an immune cell, e.g. a lymphocyte such as a T-cell, can specifically bind at least one counter-structure expressed on a second immune cells, e.g. a lymphocyte such as a T-cell. The counter-structure on the second immune cells, e.g. second T-cell, can be an inhibitory counter-structure or a stimulatory counter-structure. Exemplary counter-structures include cell surface receptors or ligands. Examples of inhibitory receptors/ligands include PD-1/PD-L1, PD-L2, CTLA-4/B7-1/B7-2, BTLA/HVEM, LAGS/MHC class II, TIGIT/PVR, and TIM-3/CEACAM-1/GAL9. Examples of stimulatory receptors/ligands include CD28/B7-1/B7-2, ICOS/ICOSL, and CD226/PVR.

In some embodiments, the TIP is expressed on a lymphocyte or an NK cell that is in trans arrangement to the mammalian cell on which is expressed the counter-structure to which it specifically binds. In alternate embodiments it is in cis arrangement. In some embodiments, the TIP specifically binds to counter-structures that are cis and trans. In a particular embodiment, the TIP is expressed on a T-cell and comprises an affinity modified IgSF domain that specifically binds to a counter-structure expressed on a T-cell. In some embodiments the first and second T-cells are separate T-cells and in this embodiment the TIP and counter-structure are in trans to each other. In some embodiments, the TIP and counter-structure are expressed on the same T-cell and are cis to each other. In some embodiments, the TIP and counter-structure to which it specifically binds can be both cis and trans. In some embodiments, at least one of the T-cells is a native T-cell or an engineered T-cell. In some embodiments, the engineered T-cell is a chimeric antigen receptor (CAR) T-cell or a T-cell receptor (TCR) engineered T-cell.

In some embodiments, a transmembrane immunomodulatory protein comprises an affinity modified IgSF domain with increased affinity to a cell surface receptor to stimulate an increase in receptor signal transduction. Stimulating an increase in receptor signaling can in some embodiments increase immunological activity of that cell if, for example, the receptor is a stimulatory receptor that works to mediate those effects. In some cases, the inflammatory activity of the cell in which receptor signaling is stimulated is increased. In some embodiments, the transmembrane immunomodulatory protein increases the activity of a stimulatory receptor. In such examples, an IgSF domain of a transmembrane immunomodulatory protein can be affinity modified to increase the specific binding affinity to the native counter-structure on a mammalian cell, which, in some cases, is a stimulatory receptor. In some embodiments, the stimulatory receptor is a expressed on T cells. In certain embodiments, the affinity modified IgSF domain of the TIP, such as is expressed by a TIP-engineered cell (e.g. a first T cell), specifically binds to a stimulatory counter-structure expressed on a T cell (e.g. a second T cell) with increased affinity (relative to the non-affinity modified IgSF domain as a control). In certain embodiments, the affinity modified IgSF domain of the TIP specifically binds to a stimulatory counter-structure expressed on a T cell (e.g. second T cell) and increases immunomodulatory activity of the T-cell. In some embodiments, the affinity modified IgSF domain of a TIP binds to a stimulatory counter-structure on the T cell (e.g. second T-cell) with increased affinity and increases immunomodulatory activity of the T-cell.

In some embodiments, the stimulatory receptor is CD28, ICOS or CD226 and the transmembrane immunomodulatory protein is one that contains an ectodomain comprising an affinity-modified IgSF domain that exhibits increased binding affinity to one of CD28, ICOS or CD226 compared to a transmembrane protein containing a wild-type IgSF domain. In some embodiments, the affinity modified IgSF domain is an affinity modified domain of B7-1 (CD80). In some embodiments, an affinity modified CD80 (B7-1) IgSF domain of a TIP of the present invention is expressed on a first T-cell and is affinity modified to bind with increased affinity to the stimulatory counter-structure CD28 on the second T-cell. In some embodiments, the affinity modified IgSF domain is an affinity modified domain of ICOSL. In specific embodiments, the affinity modified IgSF domain is an affinity modified ICOSL (inducible costimulator ligand) domain and the stimulatory counter-structure is at least one of: ICOS (inducible costimulator) or CD28. In some embodiments, the ICOSL domain is affinity modified to specifically bind to both ICOS and CD28. In some embodiments, ICOSL is affinity modified to specifically bind to either ICOS or to CD28 but not both. In some embodiments, binding affinity to one of ICOS or CD28 is increased while binding affinity to the other is attenuated. In some embodiments, the affinity modified IgSF domain is an affinity modified CD155. In some embodiment, the affinity modified IgSF domain is an affinity modified CD112.

In some methods of the present invention, the transmembrane immunomodulatory protein attenuates the activity of an inhibitory receptor. In some cases, the increased binding affinity of the transmembrane immunomodulatory protein to a cognate cell surface molecule results in inhibition of specific binding between native counter-structures on mammalian cells. The greater affinity for that native counter-structure (relative to the competing affinity of the native IgSF member) attenuates specific binding affinity of native molecule to its counter-structure. Those of skill in the art will appreciate that antagonizing an inhibitory receptor signaling can in some embodiments attenuate immunological activity of that cell if, for example, the receptor is an inhibitory receptor that serves to cause those cellular effects.

Thus, in some embodiments, a TIP can be used to stimulate a cell on which the TIP is not expressed (i.e., the trans cell) while attenuating inhibition of the cell on which the TIP is expressed (the cis cell). For example, in some embodiments, the TIP comprises at least one affinity modified domain, and in some cases at least two affinity modified domains, that results in increased binding affinity to at least two cell surface cognate binding partners. In some embodiments, a first cognate binding partner is a stimulatory receptor and the second cell surface cognate binding partner is an inhibitory ligand of an inhibitory receptor. In some embodiments, binding of the affinity-modified domain to the inhibitory ligand competitively inhibits binding of the inhibitory ligand to the inhibitory receptor. In some embodiments, the stimulatory receptor and inhibitory receptor can independently be expressed on immune cells, such as T cells or antigen presenting cells. In some embodiments the stimulatory receptor is expressed on lymphocytes, such as T cells. In some embodiments, the inhibitory receptor is expressed on the TIP-engineered cells, such as an engineered T-cell. In some embodiments, the inhibitory receptor is PD-1, CTLA-4, LAG-3, TIGIT, CD96, CD112R, BTLA, CD160 or TIM-3 and/or the ligand of the inhibitory receptor is PD-L1, PD-L2, B7-1, B7-2, HVEM, MHC class II, PVR, CEACAM-1 or GAL9 (see e.g. Table 1). In some embodiments, the inhibitory counter-structure (i.e. inhibitory ligand and inhibitory receptor) is PD-L1 or PD-1.

In some embodiments, a TIP can be used to attenuate inhibition of the cell on which the TIP is expressed, such as a T cell in which the TIP is expressed. For example, a TIP expressed on a T cell (e.g. first T-cell) can comprise an affinity modified IgSF domain that inhibits specific binding between a counter-structure on a second T-cell and an inhibitory counter-structure (i.e. inhibitory receptor) expressed on the first T-cell. In some cases, this embodiment can be used independently or in conjunction with embodiments wherein an affinity modified IgSF domain of the invention is expressed on a first T-cell and specifically binds at least one stimulatory counter-structure expressed on a second T-cell and increases immunological activity in the second T-cell. By this mechanism, an increased immunomodulatory response is generated in the second T-cell by specific binding of the TIP expressed on the first T-cell to a stimulatory counter-structure on the second cell; and the second T-cell is inhibited from attenuating the immunomodulatory activity of the first T-cell by specific binding of an affinity modified IgSF domain expressed on the first T-cell that inhibits specific binding by and between a counter-structure on the second T-cell and an inhibitory counter-structure expressed on the first T-cell. The T-cells used in this and the preceding embodiments are generally murine or human T-cells although other mammalian T-cells can be employed. Often, cytotoxic T-cells (CTL) are used.

As previously noted, in some embodiments a TIP of the present invention is expressed on a first T-cell and comprises an affinity modified IgSF domain that specifically binds to a stimulatory counter-structure (e.g. stimulatory receptor) on a second T-cell while also inhibiting specific binding between a native counter-structure (e.g. inhibitory ligand) on the second T-cell to its inhibitory native counter-structure (e.g. inhibitory receptor) on the first T-cell. Inhibition of specific binding between the counter-structure on the second T-cell to the counter-structure on the first T-cell can be achieved by competitive binding of an affinity modified IgSF domain with at least one of the two native counter-structures such that their mutual binding is interfered with. Typically, the IgSF domain is affinity modified to have a higher binding affinity to its counter-structure than the native counter-structures have to each other. In some embodiments of this design, a TIP can comprise an affinity modified IgSF domain that binds to both the inhibitory and stimulatory counter-structures. Thus, in this embodiment the affinity-modified IgSF has dual binding capability. In some embodiments, a TIP comprises a first affinity-modified IgSF domain that binds a counter-structure on the first T-cell and a second affinity-modified IgSF domain that inhibits specific binding by and between the counter-structures on the first and second T-cells.

In yet another embodiment, the affinity modified IgSF domain that binds to the stimulatory counter-structure on the first T-cell is on a first TIP and the affinity modified IgSF domain that inhibits specific binding by and between the counter-structures on the first and second T-cells is on a second TIP. In this embodiment, the first and second TIP are different polypeptide chains. In some embodiments, the first affinity-modified IgSF domain and the second affinity-modified IgSF domain are the identical affinity-modified IgSF domain. For example, in a specific embodiment the ICOSL (inducible costimulator ligand) IgSF domain (e.g. affinity modified IgV domain) is affinity modified to specifically bind with increased affinity to both ICOS and CD28. In some embodiments, the affinity modified IgSF domain is an affinity modified ICOSL IgSF domain (e.g. affinity modified IgV domain) with increased affinity to both ICOS and CD28, or decreased affinity to one of or both of: ICOS and CD28.

In some embodiments, the transmembrane immunomodulatory proteins results in inhibition of specific binding by and between native counter-structures. In some embodiments, this can be achieved by an affinity-modified IgSF domain having greater affinity for one or both native counter-structures thereby competitively inhibiting the specific binding by and between these counter-structures.

In some embodiments, the TIP comprises an affinity modified IgSF domain that is an affinity modified CD155 IgSF domain with increased affinity to CD226 and attenuated affinity to TIGIT (T-cell immunoreceptor with Ig and ITIM domains).

In some embodiments, the TIP (e.g. expressed on a first T cell) comprises an affinity modified CD80 (97-1) IgSF domain that is affinity modified to bind with increased affinity to the stimulatory counter-structure CD28 (e.g. on a second T-cell). Additionally, in this embodiment, the affinity modified CD80 (97-1) domain can bind with increased affinity to PD-L1 (e.g. expressed on the second T-cell) and inhibit specific binding to its PD-1 counter-structure (e.g. expressed on the first T-cell). In yet a further addition either of the preceding embodiments, the affinity modified CD80 (B7-1) domain can be affinity modified such that it does not substantially specifically bind to CTLA-4 or binds with attenuated affinity and therefore is not significantly inhibited in its specific binding to the stimulatory counter-structure CD28 by CTLA-4.

In some embodiments, a transmembrane immunomodulatory protein (TIP) can be used as a decoy counter-structure to inhibit specific binding by and between native counter-structures, at least one of which comprises an IgSF family member. In some cases, specific binding of a TIP comprising an affinity modified IgSF domain with one of the native counter-structures inhibits mutual specific binding by and between the native counter-structures (e.g. native receptor and ligand pairs). Thus, in some embodiments, TIPs can attenuate specific binding by means of competitive or non-competitive binding. In some embodiments, the native counter-structure is a cell surface receptor, which can be a stimulatory receptor or an inhibitory receptor. Embodiments wherein specific binding of the affinity modified IgSF domain of a TIP increases or attenuates immunological activity of the T-cell are included within the scope of the invention.

In some embodiments, a native counter-structure is an inhibitory counter-structure that acts to attenuate immunological activity when specifically bound by its native counter-structure. For example, a native cell surface counter-structure expressed on an antigen presenting cell (APC) or a mammalian tumor cell can specifically bind a native inhibitory counter-structure on an NK cell or a lymphocyte such as a T-cell. Specific binding to the inhibitory counter-structure acts to attenuate immunomodulatory activity of the NK cell or lymphocyte on which the inhibitory counter-structure is expressed.

In some embodiments, an inhibitory counter-structure is an inhibitory receptor. In some embodiments, the inhibitory counter-structure is an ITIM (immunoreceptor tyrosine-based inhibition motif) containing inhibitory counter-structure. The ITIM motif is found in the endodomain of many inhibitory receptors of the immune system (Cell Signal, 16 (4): 435-456, 2004). In some embodiments, the affinity modified domain is an affinity modified form of a wild-type inhibitory receptor that results in greater affinity of the affinity modified domain of the TIP for its native binding partner than the wild-type inhibitory receptor for the native binding partner. Thus, in these embodiments a TIP can attenuate the inhibitory response of ITIM motif receptors by specific binding of the TIP affinity modified IgSF domain to its native IgSF domain counter-structure, such as specific binding of the TIP affinity modified IgSF domain to the ITIM containing inhibitory receptor. As an example, an ITIM containing counter-structure is PD-1. Typically, PD-1 is the inhibitory receptor that is specifically bound to the inhibitory ligand PD-1. Upon specific binding of PD-L1 to PD-1, PD-1 is involved in inhibiting T-cell activation via signal transduction from the ITIM domain.

In some embodiments, the inhibitory receptor counter-structure is PD-1, CTLA-4, LAG3, TIGIT, TIM-3, or BTLA. In some embodiments, the TIP contains an affinity-modified domain that is an affinity-modified IgSF domain of PD-1, CTLA-4, LAG3, TIGIT, TIM-3, or BTLA that binds with greater affinity to the native inhibitory ligand of the inhibitory receptor than the wild-type inhibitory receptor (see Table 1 for ligand binding partners of exemplary inhibitory receptors). In some embodiments, a TIP can comprise an affinity modified PD-1 IgSF domain that binds with greater affinity to PD-L1 than wild-type PD-1. Specific binding can be achieved by competitive or non-competitive binding and are specific embodiments of the invention. Competitive binding by and between the affinity modified IgSF domain and the counter-structure (i.e. inhibitory receptor, e.g. PD-1) inhibits its binding to its native ligand counter-structure (e.g., PD-L1). In some embodiments, the TIP of this embodiment substantially lacks the signal transduction mechanism of the wild-type inhibitory receptor and therefore does not itself induce an inhibitory response.

### IV. COMPOSITIONS, METHODS AND THERAPEUTIC APPLICATIONS

Provided herein are compositions and methods related to the provided transmembrane immunomodulatory proteins and engineered cells thereof for use in modulating immunological activity of a mammalian cell. The compositions can be used in associated methods to, for example, modulate immunological activity in an immunotherapy approach to the treatment of, for example, a mammalian cancer or, in other embodiments the treatment of autoimmune disorders. The methods employed generally comprise a method of contacting a TIP of the present invention with a mammalian cell under conditions that are permissive to specific binding of the affinity modified IgSF domain and modulation of the immunological activity of the mammalian cell. The methods can be employed ex vivo or in vivo. In some embodiments, the method of modulating immunological activity is achieved by expression of a TIP of the present invention on a lymphocyte (e.g., a T-cell or TIL) or NK cell engineered to express the TIP. The cell expressing the TIP is contacted with a mammalian cell such as an APC, a second lymphocyte or tumor cell under conditions that are permissive of specific binding of the affinity modified IgSF domain to a counter-structure on the mammalian cell such that immunological activity can be modulated in the mammalian cell. In some embodiments, the methods is conducted by adoptive cell transfer wherein cells expressing the TIP (e.g., a T-cell) are infused back into the patient.

Provided herein are methods of administering a therapeutic amount of a cell composition containing the provided transmembrane immunomodulatory proteins to a subject having a disease or disorder. The pharmaceutical compositions described herein can be used in a variety of therapeutic applications, such as the treatment of a disease. For example, in some embodiments the pharmaceutical composition is used to treat inflammatory or autoimmune disorders, cancer, organ transplantation, viral infections, and/or bacterial infections in a mammal. The pharmaceutical composition can modulate an immune response to treat the disease. For example, in some embodiments, the pharmaceutical composition stimulates the immune response, which can be useful, for example, in the treatment of cancer, viral infections, or bacterial infections. In some embodiments, the pharmaceutical composition suppresses an immune response, which can be useful in the treatment of inflammatory or autoimmune disorders, or organ transplantation.

The provided methods are believed to have utility in a variety of applications, including, but not limited to, e.g., in prophylactic or therapeutic methods for treating a variety of immune system diseases or conditions in a mammal in which modulation or regulation of the immune system and immune system responses is beneficial. For example, suppressing an immune response can be beneficial in prophylactic and/or therapeutic methods for inhibiting rejection of a tissue, cell, or organ transplant from a donor by a recipient. In a therapeutic context, the mammalian subject is typically one with an immune system disease or condition, and administration is conducted to prevent further progression of the disease or condition.

Cell compositions expressing transmembrane immunomodulatory proteins of the present invention and associated methods can be used in immunotherapy applications. In some embodiments, engineered cells for expression of a transmembrane immunomodulatory protein (TIP) are cells isolated from a mammal, such as a mouse or human. In some embodiments, the mammalian cell serving as a host cell for expression of a TIP is a lymphocyte such as a tumor infiltrating lymphocyte (TIL), a natural killer (NK) cell, or a T-cell such as a CD8+ cytotoxic T lymphocyte or a CD4+ helper T lymphocyte. In some embodiments, the cells are autologous cells. In aspects of the provided method, the engineered cells are contacted, generally under physiological conditions, with a mammalian cell in which modulation of immunological activity is desired. For example, the mammalian cell can be a murine or human cell such as an antigen presenting cell or tumor cell. In some embodiments, the engineered cells are autologous cells. In other embodiments, the cells are allogeneic. Cells can be contacted in vivo or ex vivo. In some embodiments, the engineered cells are administered to the subject, such as by infusion. Thus, composition and methods can be used in adoptive cell transfer immunotherapy.

In some embodiments, an effective amount of a pharmaceutical composition is administered to inhibit, halt, or reverse progression of cancers that are sensitive to modulation of immunological activity by transmembrane immunomodulatory proteins of the present invention. In some embodiments, the methods of the invention are used in the treatment of a mammalian patient of cancers such as lymphoma, lymphoid leukemia, myeloid leukemia, cervical cancer, neuroblastoma, or multiple myeloma. Other cancers which can be treated by the methods of the invention include, but are not limited to, melanoma, bladder cancer, hematological malignancies (leukemia, lymphoma, myeloma), liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, or Ewing's sarcoma.

Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient. Thus, the present invention provides ex vivo and in vivo methods to inhibit, halt, or reverse progression of the tumor, or otherwise result in a statistically significant increase in progression-free survival (i.e., the length of time during and after treatment in which a patient is living with cancer that does not get worse), or overall survival (also called “survival rate;” i.e., the percentage of people in a study or treatment group who are alive for a certain period of time after they were diagnosed with or treated for cancer) relative to treatment with a control.

In some embodiments, a pharmaceutical composition of the invention can also be used to inhibit growth of mammalian, particularly human, cancer cells as a monotherapy (i.e., as a single agent), in combination with at least one chemotherapeutic agent (i.e., a combination therapy), in combination with a cancer vaccine, in combination with an immune checkpoint inhibitor and/or in combination with radiation therapy. In some aspects of the present disclosure, the immune checkpoint inhibitor is nivolumab, tremelimumab, pembrolizumab, ipilimumab, or the like.

In some embodiments, the provided compositions can attenuate an immune response, such as, for example, where the transmembrane immunomodulatory protein comprises an affinity modified IgSF domain of an inhibitory ligand. In some embodiments, the compositions can be used to treat an autoimmune disease. In some embodiments, the administration of a therapeutic composition of the invention to a subject suffering from an immune system disease (e.g., autoimmune disease) can result in suppression or inhibition of such immune system attack or biological responses associated therewith. By suppressing this immune system attack on healthy body tissues, the resulting physical symptoms (e.g., pain, joint inflammation, joint swelling or tenderness) resulting from or associated with such attack on healthy tissues can be decreased or alleviated, and the biological and physical damage resulting from or associated with the immune system attack can be decreased, retarded, or stopped. In a prophylactic context, the subject may be one with, susceptible to, or believed to present an immune system disease, disorder or condition, and administration is typically conducted to prevent progression of the disease, disorder or condition, inhibit or alleviate symptoms, signs, or biological responses associated therewith, prevent bodily damage potentially resulting therefrom, and/or maintain or improve the subject's physical functioning.

In some embodiments, the pharmaceutical compositions comprising cells engineered with TIPs can be used to treat one or more other immune disease or disorder in the subject. The immune system disease or disorder of the patient may be or involve, e.g., but is not limited to, Addison's Disease, Allergy, Alopecia Areata, Alzheimer's, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barré syndrome), Churg-Strauss Syndrome (CSS), Cicatricial Pemphigoid, Cold Agglutinin Disease (CAD), COPD, CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, graft-related disease or disorder, Graves′Disease, GVHD, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Ménèire's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjörgen's Syndrome, Solid-organ transplant rejection (kidney, heart, liver, lung, etc.), Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, Wegener's Granulomatosis, and preventing or suppressing an immune response associated with rejection of a donor tissue, cell, graft, or organ transplant by a recipient subject. Graft-related diseases or disorders include graft versus host disease (GVDH), such as associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc. With regard to a donor tissue, cell, graft or solid organ transplant in a recipient subject, it is believed that a therapeutic composition of the invention disclosed herein may be effective in preventing acute rejection of such transplant in the recipient and/or for long-term maintenance therapy to prevent rejection of such transplant in the recipient (e.g., inhibiting rejection of insulin-producing islet cell transplant from a donor in the subject recipient suffering from diabetes).

In some embodiments, a therapeutic amount of the pharmaceutical composition is administered. Typically, precise amount of the compositions of the present invention to be administered can be determined by a physician with consideration of individual differences in age, weight, tumor size, extent of infection or metastasis, and condition of the patient (subject). It can generally be stated that a pharmaceutical composition comprising engineered cells, e.g. T cells, as described herein may be administered at a dosage of 10⁴ to 10⁹ cells/kg body weight, such as 10⁵ to 10⁶ cells/kg body weight, including all integer values within those ranges. Engineered cell compositions, such as T cell compositions, may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the therapeutic composition is administered to a patient by intradermal or subcutaneous injection. In another embodiment, the therapeutic compositions is administered by i.v. injection. In some cases, the cell compositions may be injected directly into a tumor, lymph node, or site of infection.

### Pharmaceutical Formulation

Provided are pharmaceutical compositions containing the transmembrane immunomodulatory proteins, including engineered cells expressing such transmembrane immunomodulatory proteins. In some embodiments, the pharmaceutical compositions and formulations include one or more optional pharmaceutically acceptable carrier or excipient.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

Such a formulation may, for example, be in a form suitable for intravenous infusion. A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect. The pharmaceutical composition of the invention can be administered parentally, subcutaneously, or intravenously, or as described elsewhere herein. The pharmaceutical composition of the invention may be administered in a therapeutically effective amount one, two, three or four times per month, two times per week, biweekly (every two weeks), or bimonthly (every two months). Administration may last for a period of 1, 2, 3, 4, 5, 6, 7, 8, 9, 10, 11, or 12 months or longer (e.g., one, two, three, four or more years, including for the life of the subject).

For any composition, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the cell composition or to maintain the desired effect. Factors that may be taken into account include the severity of the disease state, the general health of the subject, the age, weight, and gender of the subject, time and frequency of administration, drug combination(s), reaction sensitivities, and response to therapy. Appropriate dosages may be ascertained through use of appropriate dose-response data. A number of biomarkers or physiological markers for therapeutic effect can be monitored including T cell activation or proliferation, cytokine synthesis or production (e.g., production of TNF-α, IFN-γ, IL-2), induction of various activation markers (e.g., CD25, IL-2 receptor), inflammation, joint swelling or tenderness, serum level of C-reactive protein, anti-collagen antibody production, and/or T cell-dependent antibody response(s).

A variety of means are known for determining if administration of a therapeutic composition of the invention sufficiently modulates immunological activity by eliminating, sequestering, or inactivating immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of immunological activity include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T cell proliferation assays and pepscan analysis based on 3H-thymidine incorporation following stimulation with anti-CD3 antibody, anti-T cell receptor antibody, anti-CD28 antibody, calcium ionophores, PMA, antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimensional gel electrophoresis, Western blot analysis, enzyme linked immunosorbent assays, flow cytometry); and measurement of clinical symptoms or outcomes such as improvement of autoimmune, neurodegenerative, and other diseases involving self proteins or self polypeptides (clinical scores, requirements for use of additional therapies, functional status, imaging studies) for example, by measuring relapse rate or disease severity (using clinical scores known to the ordinarily skilled artisan) in the case of multiple sclerosis, measuring blood glucose in the case of type I diabetes, or joint inflammation in the case of rheumatoid arthritis.

### V. EXEMPLARY EMBODIMENTS

Among the provided embodiments are:

1. A transmembrane immunomodulatory protein (TIP) comprising:
(i) an ectodomain comprising at least one non-immunoglobulin affinity-modified immunoglobulin superfamily (IgSF) domain comprising one or more amino acid substitution(s) in a wild-type IgSF domain, wherein the at least one affinity-modified IgSF domain specifically binds at least one cell surface cognate binding partner of the wild-type IgSF domain; and
(ii) a transmembrane domain.

2. The transmembrane immunomodulatory protein of embodiment 1, wherein the at least one cell surface cognate binding partner is expressed on a mammalian cell.

3. The transmembrane immunomodulatory protein of embodiment 2, wherein the mammalian cell is an antigen presenting cell (APC), a tumor cell, or a lymphocyte, which optionally is a T-cell.

4. The transmembrane immunomodulatory protein of any of embodiments 1-3, wherein the mammalian cell is a mouse, rat, cynomolgus monkey, or human cell.

5. The transmembrane immunomodulatory protein of any of embodiments 1-4, wherein the at least one affinity modified IgSF domain has increased binding affinity to the at least one cell surface cognate binding partner compared with the reference wild-type IgSF domain.

6. The transmembrane immunomodulatory protein of any of embodiments 2-5, wherein specific binding of the transmembrane immunomodulatory protein comprising the at least one affinity-modified IgSF domain modulates immunological activity of the mammalian cell compared with the reference transmembrane domain comprising the wild-type IgSF domain.

7. The transmembrane immunomodulatory protein of any of embodiments 2-6, wherein specific binding of the transmembrane immunomodulatory protein comprising the at least one affinity-modified IgSF domain increases immunological activity of the mammalian cell compared with the reference transmembrane domain comprising the wild-type IgSF domain.

8. The transmembrane immunomodulatory protein of any of embodiments 2-6, wherein specific binding of the transmembrane immunomodulatory protein attenuates immunological activity of the mammalian cell compared with the reference transmembrane domain comprising the wild-type IgSF domain.

9. The transmembrane protein of any of embodiments 1-8, wherein the wild-type IgSF domain is from an IgSF family member of a family selected from Signal-Regulatory Protein (SIRP) Family, Triggering Receptor Expressed On Myeloid Cells Like (TREML) Family, Carcinoembryonic Antigen-related Cell Adhesion Molecule (CEACAM) Family, Sialic Acid Binding Ig-Like Lectin (SIGLEC) Family, Butyrophilin Family, B7 family, CD28 family, V-set and Immunoglobulin Domain Containing (VSIG) family, V-set transmembrane Domain (VSTM) family, Major Histocompatibility Complex (MHC) family, Signaling lymphocytic activation molecule (SLAM) family, Leukocyte immunoglobulin-like receptor (LIR), Nectin (Nec) family, Nectin-like (NECL) family, Poliovirus receptor related (PVR) family, Natural cytotoxicity triggering receptor (NCR) family, T cell immunoglobulin and mucin (TIM) family or Killer-cell immunoglobulin-like receptors (KIR) family.

10. The transmembrane immunomodulatory protein of any of embodiments 1-9, wherein the wild-type IgSF domain is from an IgSF member selected from CD80, CD86, PD-L1, PD-L2, ICOS Ligand, B7-H3, B7-H4, CD28, CTLA4, PD-1, ICOS, BTLA, CD4, CD8-alpha, CD8-beta, LAG3, TIM-3, CEACAM1, TIGIT, PVR, PVRL2, CD226, CD2, CD160, CD200, CD200R or Nkp30.

11. The transmembrane immunomodulatory protein of any of embodiments 1-10, wherein the wild-type IgSF domain is a human IgSF member.

12. The transmembrane immunomodulatory protein of any of embodiments 1-11, wherein the at least one affinity modified IgSF domain has at least 90% sequence identity to a wild-type IgSF domain or a specific binding fragment thereof contained in the sequence of amino acids set forth in any of SEQ ID NOS: 1-54.

13. The transmembrane immunomodulatory protein of any of embodiments 1-12, wherein the transmembrane immunomodulatory protein has at least 90% sequence identity to the amino acid sequence selected from any of SEQ ID NOS: 393-419.

14. The transmembrane immunomodulatory protein of any of embodiments 1-13, wherein the at least one cell surface cognate binding partner is a stimulatory receptor expressed on a T-cell and the at least one affinity-modified IgSF domain has increased binding affinity to the stimulatory receptor compared to the affinity of the wild-type IgSF domain.

15. The transmembrane immunomodulatory protein of embodiment 14, wherein binding of the affinity-modified IgSF domain to the stimulatory receptor increases immunological activity of the T-cell.

16. The transmembrane immunomodulatory protein of embodiment 14 or embodiment 15, wherein the stimulatory receptor is CD28, ICOS or CD226.

17. The transmembrane immunomodulatory protein of any one of embodiments 14-16, wherein the at least one affinity-modified IgSF domain is an affinity modified B7-1 IgSF domain o and the stimulatory receptor is CD28.

18. The transmembrane immunomodulatory protein of any one of embodiments 14-16, wherein the at least one affinity-modified IgSF domain is an affinity modified ICOSL IgSF domain o and the stimulatory receptor is ICOS.

19. The transmembrane immunomodulatory protein of any one of embodiments 14-16, wherein the affinity-modified IgSF domain is an affinity modified ICOSL IgSF domain and the stimulatory receptor is CD28.

20. The transmembrane immunomodulatory protein of any one of embodiments 14-16, 18 and 19, wherein the at least one affinity-modified IgSF domain is an affinity-modified ICOSL IgSF domain that has increased binding affinity to at least one of: ICOS and CD28.

21. The transmembrane immunomodulatory protein of any one of embodiments 14-16- and 18-20, wherein the affinity modified IgSF domain is an affinity modified ICOSL IgV IgSF domain with increased binding affinity to both ICOS and CD28.

22. The transmembrane immunomodulatory protein of any one of embodiments 17-21, wherein the affinity-modified IgSF domain does not substantially specifically bind to CTLA-4 or exhibits decreased binding affinity to CTLA-4 compared to the wild-type IgSF domain.

23. The transmembrane immunomodulatory protein of any of embodiments 1-22, wherein the at least one affinity-modified IgSF domain specifically binds to no more than one cell surface cognate binding partner.

24. The transmembrane immunomodulatory protein of any of embodiments 1-23, wherein the transmembrane immunomodulatory protein specifically binds to no more than one cell surface cognate binding partner.

25. The transmembrane immunomodulatory protein of any of embodiments 1-22, wherein the at least one affinity-modified domain specifically binds to at least two cell surface cognate binding partners.

26. The transmembrane immunomodulatory protein of embodiment 25, wherein:

the first cell surface cognate binding partner is a stimulatory receptor expressed on a T cell; and

the second cell surface cognate binding partner is an inhibitory ligand of an inhibitory receptor, wherein the inhibitory receptor is expressed on a T-cell.

27. The transmembrane immunomodulatory protein of embodiment 26, wherein binding of the affinity-modified domain to the inhibitory ligand competitively inhibits binding of the inhibitory ligand to the inhibitory receptor.

28. The transmembrane immunomodulatory protein of embodiment 26 or embodiment 27, wherein:

the inhibitory receptor is PD-1, CTLA-4, LAG-3, TIGIT, CD96, CD112R, BTLA, CD160 or TIM-3; or

the ligand of the inhibitory receptor is PD-L1, PD-L2, B7-1, B7-2, HVEM, MHC class II, PVR, CEACAM-1 or GAL9.

29. The transmembrane immunomodulatory protein of any one of embodiments 26-28, wherein the affinity modified IgSF domain is an affinity modified B7-1 domain and the stimulatory receptor is CD28.

30. The transmembrane immunomodulatory protein of embodiment 29, wherein the inhibitory ligand is PD-L1 and the inhibitory receptor is PD-1.

31. The transmembrane immunomodulatory protein of embodiment 29 or embodiment 30, wherein the affinity-modified IgSF domain exhibits decreased binding affinity to CTLA-4 compared to the wild-type IgSF domain for CTLA-4.

32. The transmembrane immunomodulatory protein of any one of embodiments 29-31, wherein the affinity-modified IgSF domain does not substantially specifically bind to CTLA-4.

33. The transmembrane immunomodulatory protein of any of embodiments 1-13, wherein the affinity modified IgSF domain is an affinity modified CD155 IgSF domain or an affinity modified CD112 IgSF domain and the stimulatory receptor is CD226.

34. The transmembrane immunomodulatory protein of embodiment 33, wherein the affinity-modified IgSF domain exhibits decreased binding affinity to TIGIT (T-cell immunoreceptor with Ig and ITIM domains) compared to the affinity of the wild-type IgSF domain.

35. The transmembrane immunomodulatory protein of any of embodiments 1-13, wherein the at least one affinity-modified IgSF domain specifically binds to a cell surface cognate binding partner that is a tumor specific antigen.

36. The transmembrane immunomodulatory protein of embodiment 35, wherein the tumor specific antigen is B7-H6.

37. The transmembrane immunomodulatory protein of embodiment 35 or embodiment 36, wherein the affinity modified IgSF domain is an affinity modified Nkp30 IgSF domain.

38. The transmembrane immunomodulatory protein of any one of embodiments 1-37, wherein the at least one affinity-modified IgSF domain is a first affinity-modified IgSF domain and the ectodomain comprises a second affinity-modified IgSF domain.

39. The transmembrane immunomodulatory protein of embodiment 38, wherein the first and second affinity-modified IgSF domain are different.

40. The transmembrane immunomodulatory protein of embodiment 38 or embodiment 39, wherein the first affinity-modified IgSF domain and the second affinity-modified IgSF domain each comprise one or more amino acid different substitutions in the same wild-type IgSF domain.

41. The transmembrane immunomodulatory protein of embodiment 38 or embodiment 39, wherein the first affinity-modified IgSF domain and the second affinity-modified IgSF domain each comprise one or more amino acid substitutions in a different wild-type IgSF domain.

42. The transmembrane immunomodulatory protein of any of embodiments 1-41, wherein the transmembrane immunomodulatory protein further comprises an endodomain or cytoplasmic signaling domains.

43. The transmembrane immunomodulatory protein of embodiment 42, wherein the endodomain is the endodomain from the wild-type IgSF member comprising the wild-type IgSF domain or is a functionally active portion thereof.

44. The transmembrane immunomodulatory protein of embodiment 42, wherein the transmembrane immunomodulatory protein is a chimeric receptor, wherein the endodomain is not the endodomain from the wild-type IgSF member comprising the wild-type IgSF domain.

45. The transmembrane immunomodulatory protein of embodiment 42 or embodiment 44, wherein the endodomain comprises at least one ITAM (immunoreceptor tyrosine-based activation motif)-containing signaling domain.

46. The transmembrane immunomodulatory protein of any of embodiments 42, 44 and 45, wherein the endodomain comprises a CD3-zeta signaling domain.

47. The transmembrane immunomodulatory protein of embodiment 45 or embodiment 46, wherein the endodomain further comprises at least one of: a CD28 costimulatory domain, an ICOS signaling domain, an OX40 signaling domain, and a 41BB signaling domain.

48. The transmembrane immunomodulatory protein of any of embodiments 1-13, wherein the wild-type IgSF domain is from an IgSF member that is an inhibitory receptor comprising an ITIM signaling domain.

49. The transmembrane immunomodulatory protein of embodiment 48, wherein the inhibitory receptor is PD-1, CTLA-4, LAG3, TIGIT, TIM-3, or BTLA and the at least one affinity-modified IgSF domain is an affinity-modified IgSF domain of PD-1, CTLA-4, LAG3, TIGIT, TIM-3, or BTLA, respectively.

50. The transmembrane immunomodulatory protein of embodiment 48 or embodiment 49, wherein the inhibitory receptor is PD-1 and the at least one affinity-modified IgSF domain is an affinity-modified IgSF of PD-1.

51. The transmembrane immunomodulatory protein of any of embodiments 48-50, wherein the affinity-modified IgSF domain has increased binding affinity for a trans surface cognate binding partner compared to the wildtype IgSF domain, whereby the increased binding affinity competitively inhibits binding of the trans surface cognate binding partner to the inhibitory receptor.

52. The transmembrane immunomodulatory protein of any of embodiments 48-51, wherein the transmembrane immunomodulatory protein does not comprise an endodomain, ITIM or cytoplasmic signaling domains.

53. The transmembrane immunomodulatory protein of any of embodiments 1-52, wherein the affinity modified IgSF domain differs by no more than ten amino acid substitutions from the wildtype IgSF domain.

54. The transmembrane immunomodulatory protein of any of embodiments 1-53, wherein the affinity modified IgSF domain differs by no more than five amino acid substitutions from the wildtype IgSF domain.

55. The transmembrane immunomodulatory protein of any of embodiments 1-54, wherein the affinity-modified IgSF domain is or comprises an affinity modified IgV domain, affinity modified IgC1 domain or an affinity modified IgC2 domain or is a specific binding fragment thereof comprising the one or more amino acid substitutions.

56. The transmembrane immunomodulatory protein of any of embodiments 1-55, wherein the ectodomain further comprises one or more non-affinity modified IgSF domains.

57. The transmembrane immunomodulatory protein of embodiment 56, wherein the one or more non-affinity modified IgSF domains is from a wild-type IgSF member comprising the wild-type IgSF domain.

58. The transmembrane immunomodulatory protein of any of embodiments 1-57, wherein the transmembrane domain is the native transmembrane domain from the corresponding wild-type IgSF member.

59. The transmembrane immunomodulatory protein of any of embodiments 1-57, wherein the transmembrane domain is not the native transmembrane domain from the corresponding wild-type IgSF member.

60. The transmembrane immunomodulatory protein of embodiment 59, wherein the transmembrane protein is a transmembrane protein derived from CD8.

61. A recombinant nucleic acid encoding a transmembrane immunomodulatory proteins of any of embodiments 1-60.

62. A recombinant expression vector comprising the nucleic acid of embodiment 61.

63. A recombinant host cell comprising the expression vector of embodiment 62.

64. A recombinant host cell comprising the nucleic acid of embodiment 61.

65. The recombinant host cell of embodiment 63 or embodiment 64, wherein the host cell is a mammalian host cell.

66. The recombinant host cell of any of embodiments 63-65, wherein the mammalian host cell is a human host cell.

67. An engineered cell comprising the transmembrane immunomodulatory protein of any of embodiments 1-60.

68. The engineered cell of embodiment 67, wherein the cell is an immune cell.

69. The engineered cell of embodiment 67 or embodiment 68, wherein the cell is a lymphocyte.

70. The engineered cell of embodiment 69, wherein the lymphocyte is a T cell, a B cell or an NK cell.

71. The engineered cell of any of embodiments 67-70, wherein the cell is a T cell.

72. The engineered cell of embodiment 71, wherein the T cells is CD4+ or CD8+.

73. The engineered cell of embodiment 67 or embodiment 68, wherein the cell is an antigen presenting cell.

74. The engineered cell of any of embodiments 67-73, further comprising a chimeric antigen receptor (CAR) or an engineered T-cell receptor (TCR).

75. A pharmaceutical composition comprising the cell of any of embodiments 67-74 and a pharmaceutically acceptable carrier.

76. The pharmaceutical composition of embodiment 75 that is sterile.

77. A method of modulating an immune response in a mammalian subject, comprising administering a cell of any of embodiments 67-74 or a pharmaceutical composition of embodiment 75 or embodiment 76 to the subject.

78. The method of embodiment 76 or embodiment 77, wherein modulating the immune response treats a disease or disorder in the subject.

79. The method of any of embodiments 77-78, wherein the modulated immune response is increased.

80. The method of embodiment 78 or embodiment 79, wherein the disease or disorder is a tumor.

81. The method of any of embodiments 78-80, wherein the disease or disorder is a cancer.

82. The method of any of embodiments 78-81, wherein the disease or disorder is melanoma, lung cancer, bladder cancer, or a hematological malignancy.

83. The method of any of embodiments 77-78, wherein the modulated immune response is decreased.

84. The method of embodiment 78 or embodiment 83, wherein the disease or disorder is an inflammatory disease or condition.

85. The method of any of embodiments 78, 83 and 84, wherein the disease or condition is Crohn's disease, ulcerative colitis, multiple sclerosis, asthma, rheumatoid arthritis, or psoriasis.

86. The method of any of embodiments 77-85, wherein the subject is human.

87. The method of any of embodiments 77-86, wherein the cell is autologous to the subject.

88. The method of any of embodiments 77-87, wherein the cell is allogenic to the subject.

89. A transmembrane immunomodulatory protein (TIP) comprising:
an ectodomain, wherein the ectodomain comprises at least one non-immunoglobulin affinity-modified immunoglobulin superfamily (IgSF) domain; and
a transmembrane domain, wherein: the TIP is expressed on a first T-cell; the affinity-modified IgSF domain specifically binds at least one counter-structure expressed on a mammalian cell; the mammalian cell is an antigen presenting cell (APC), a tumor cell, or a second T-cell; and specific binding of the affinity-modified IgSF domain to a counter-structure modulates immunological activity of the mammalian cell.

90. The transmembrane immunomodulatory protein (TIP) of embodiment 89, wherein:
the TIP comprises a first affinity-modified IgSF domain, wherein the counter-structure expressed on the mammalian cell is a stimulatory counter-structure expressed on the second T-cell; and
the first affinity-modified IgSF domain specifically binds to the stimulatory counter-structure and increases immunomodulatory activity of the second T-cell.

91. The transmembrane immunomodulatory protein of embodiment 90, further comprising a second affinity-modified IgSF domain expressed on the first T-cell that competitively inhibits specific binding of a counter-structure expressed on the second T-cell to its inhibitory counter-structure expressed on the first T-cell.

92. The transmembrane immunomodulatory protein of embodiment 91, wherein the first affinity modified IgSF domain is an affinity modified B7-1 domain and the stimulatory counter-structure is CD28.

93. The transmembrane immunomodulatory protein of embodiment 92, wherein the counter-structure expressed on the second T-cell is PD-L1 and the inhibitory counter-structure expressed on the first T-cell is PD-1.

94. The transmembrane immunomodulatory protein (TIP) according to embodiments 92 or 93, wherein the first affinity-modified IgSF domain does not substantially specifically bind to CTLA-4.

95. The transmembrane immunomodulatory protein of embodiment 90, wherein the first affinity-modified IgSF domain is an affinity modified ICOSL domain and the stimulatory counter-structure is ICOS.

96. The transmembrane immunomodulatory protein of embodiment 90, wherein the first affinity-modified IgSF domain is an affinity modified ICOSL domain and the stimulatory counter-structure is CD28.

97. The transmembrane immunomodulatory protein of embodiment 90, wherein the first affinity-modified IgSF domain has increased affinity to the stimulatory counter-structure.

98. The transmembrane immunomodulatory protein (TIP) of embodiment 91, wherein the TIP ectodomain comprises both the first affinity-modified IgSF domain and the second affinity-modified IgSF domain.

99. The transmembrane immunomodulatory protein of embodiment 98, wherein the first affinity-modified IgSF domain and the second affinity-modified IgSF domain are the identical affinity-modified IgSF domain.

100. The transmembrane immunomodulatory protein of embodiment 91, wherein at least one of the first and second T-cell is a native T-cell or an engineered T-cell.

101. The transmembrane immunomodulatory protein of embodiment 91, wherein the engineered T-cell is a chimeric antigen receptor (CAR) or a T-cell receptor (TCR) engineered T-cell.

102. The transmembrane immunomodulatory protein of embodiment 90, wherein the first affinity modified IgSF domain is an ICOSL (inducible costimulator ligand) IgV IgSF domain with increased affinity to at least one of: ICOS and CD28.

103. The transmembrane immunomodulatory protein of embodiment 90, wherein the first affinity modified IgSF domain is an affinity modified ICOSL IgV IgSF domain with increased affinity to both ICOS and CD28.

104. The transmembrane immunomodulatory protein of embodiment 91, wherein the first affinity modified IgSF domain is a CD155 IgSF domain with increased affinity to CD226 and attenuated affinity to TIGIT (T-cell immunoreceptor with Ig and ITIM domains).

105. The transmembrane immunomodulatory protein of embodiment 89, wherein the mammalian cell is an autologous cell.

106. The transmembrane immunomodulatory protein of embodiment 89, wherein the mammalian cell is an allogenic cell.

107. The transmembrane immunomodulatory protein of embodiment 89, wherein the mammalian cell is a mouse, rat, cynomolgus monkey, or human cell.

108. The transmembrane immunomodulatory protein of embodiment 89, further comprising an endodomain or cytoplasmic signaling domains.

109. The transmembrane immunomodulatory protein of embodiment 89, wherein the affinity-modified IgSF domain specifically binds to the counter-structure on the mammalian cell with increased affinity.

110. The transmembrane immunomodulatory protein of embodiment 109, wherein immunological activity of the mammalian cell is increased.

111. The transmembrane immunomodulatory protein of embodiment 89, wherein immunological activity of the mammalian cell is attenuated.

112. The transmembrane immunomodulatory protein of embodiment 89, wherein the affinity modified IgSF domain differs by no more than ten amino acid substitutions from the native IgSF domain.

113. The transmembrane immunomodulatory protein of embodiment 89, wherein the affinity modified IgSF domain differs by no more than five amino acid substitutions from the native IgSF domain.

114. The transmembrane immunomodulatory protein of embodiment 89, wherein the affinity modified IgSF domain specifically binds to no more than one counter-structure.

115. The transmembrane immunomodulatory protein (TIP) of embodiment 89, wherein the TIP specifically binds to no more than one cell surface counter-structure.

116. The transmembrane immunomodulatory protein of embodiment 89, wherein the affinity modified IgSF domain specifically binds to at least two cell surface counter-structures.

117. The transmembrane immunomodulatory protein of embodiment 89, wherein the affinity modified IgSF domain is an affinity modified IgV, IgC1, or IgC2 domain.

118. The transmembrane immunomodulatory protein of embodiment 89, wherein the affinity-modified IgSF domain has increased binding affinity for a first counter-structure expressed on a mammalian cell, whereby the increased binding affinity competitively inhibits binding of the first counter-structure to a second counter-structure.

119. The transmembrane immunomodulatory protein of embodiment 118, wherein the second counter-structure is a cell surface receptor.

120. The transmembrane immunomodulatory protein of embodiment 119, wherein the cell surface receptor is an inhibitory receptor.

121. The transmembrane immunomodulatory protein of embodiment 120, wherein the inhibitory receptor is PD-1 and the affinity-modified IgSF domain is a PD-1 IgSF domain.

122. The transmembrane immunomodulatory protein of embodiment 118, wherein the second counter-structure is PD-1, CTLA-4, LAG3, TIGIT, TIM-3, or BTLA.

123. The transmembrane immunomodulatory protein (TIP) of embodiment 118, wherein the TIP increases immunological activity of the T-cell.

124. The transmembrane immunomodulatory protein (TIP) of embodiment 89, wherein the TIP is a chimeric antigen receptor (CAR), and wherein the affinity-modified IgSF domain specifically binds to a tumor specific IgSF counter-structure.

125. The transmembrane immunomodulatory protein of embodiment 124, wherein the CAR endodomain comprises a CD3-zeta signaling domain.

126. The transmembrane immunomodulatory protein of embodiment 124, wherein the CAR endodomain comprises at least one CD3 ITAM (immunoreceptor tyrosine-based activation motif).

127. The transmembrane immunomodulatory protein of embodiment 125, wherein the CAR endodomain further comprises at least one of: a CD28 costimulatory domain, an OX40 signaling domain, and a 41BB signaling domain.

128. The transmembrane immunomodulatory protein of embodiment 124, wherein the affinity modified IgSF domain specifically binds the tumor specific antigen B7-H6.

129. The transmembrane immunomodulatory protein according to embodiments 89, 90, or 91, wherein the transmembrane immunomodulatory protein has at least 90% sequence identity with an amino acid sequence selected from SEQ ID NOS: 1-26.

130. The transmembrane immunomodulatory protein according to embodiments 89, 90, or 91, wherein the transmembrane immunomodulatory protein has at least 95% sequence identity with an amino acid sequence selected from SEQ ID NOS: 1-26.

131. A recombinant nucleic acid encoding any one of the transmembrane immunomodulatory proteins of embodiments 89 to 130.

132. A recombinant expression vector comprising a nucleic acid according to embodiment 131.

133. A recombinant host cell comprising the expression vector of embodiment 132.

134. The recombinant host cell of embodiment 133, wherein the host cell is a mammalian host cell.

135. The recombinant host cell of embodiment 133 wherein the mammalian host cell is a human host cell.

136. A method of treating a mammalian patient in need of an enhanced or inhibited immunological response by administering a therapeutically effective amount of a transmembrane immunomodulatory protein of any one of embodiments 89 to 130.

137. The method of embodiment 136, wherein the enhanced immunological response treats melanoma, lung cancer, bladder cancer, or a hematological malignancy in the patient.

138. The method of embodiment 136, wherein the inhibited immunological response treats Crohn's disease, ulcerative colitis, multiple sclerosis, asthma, rheumatoid arthritis, or psoriasis in the patient.

### VI. EXAMPLES

The following examples are included for illustrative purposes only and are not intended to limit the scope of the invention.

Examples 1-10 describe the design, creation, and screening of affinity modified CD80 (B7-1), CD86 (B7-2), ICOSL, and NKp30 immunomodulatory proteins, which are components of the immune synapse (IS) that have a demonstrated dual role in both immune activation and inhibition. These examples demonstrate that affinity modification of IgSF domains yields proteins that can act to both increase and decrease immunological activity. This work also describes the various combinations of those domains fused in pairs (i.e., stacked) to form a Type II immunomodulatory protein to achieve immunomodulatory activity. Example 11 further exemplifies such domains in a transmembrane immunomodulatory protein (TIP) format and the generation of engineered cells co-expressing the TIP with a chimeric antigen receptor (CAR).

### Example 1

### Generation of Mutant DNA Constructs of IgSF Domains

Example 1 describes the generation of mutant DNA constructs of human CD80, CD86, ICOSL and NKp30 IgSF domains for translation and expression on the surface of yeast as yeast display libraries.

A. Degenerate Libraries

For libraries that target specific residues of target protein for complete or partial randomization with degenerate codons, the coding DNAs for the extracellular domains (ECD) of human CD80 (SEQ ID NO:28), ICOSL (SEQ ID NO:32), and NKp30 (SEQ ID NO:54) were ordered from Integrated DNA Technologies (Coralville, Iowa) as a set of overlapping oligonucleotides of up to 80 base pairs (bp) in length. To generate a library of diverse variants of each ECD, the oligonucleotides contained desired degenerate codons at desired amino acid positions. Degenerate codons were generated using an algorithm at the URL: rosettadesign.med.unc.edu/SwiftLib/.

In general, positions to mutate and degenerate codons were chosen from crystal structures (CD80, NKp30) or homology models (ICOSL) of the target-ligand pairs of interest were used to identify ligand contact residues as well as residues that are at the protein interaction interface. This analysis was performed using a structure viewer available at the URL:spdbv.vital-it.ch). For example, a crystal structure for CD80 bound to CTLA4 is publicly available at the URL:www.rcsb.org/pdb/explore/explore.do?structureId=1I8L and a targeted library was designed based on the CD80::CTLA4 interface for selection of improved binders to CTLA4. However, there are no CD80 structures available with ligands CD28 and PDL1, so the same library was also used to select for binders of CD28 (binds the same region on CD80 as CTLA4) and PDL1 (not known if PDL1 binds the same site as CTLA4). The next step in library design was the alignment of human, mouse, rat and monkey CD80, ICOSL or NKp30 sequences to identify conserved residues. Based on this analysis, conserved target residues were mutated with degenerate codons that only specified conservative amino acid changes plus the wild-type residue. Residues that were not conserved were mutated more aggressively, but also including the wild-type residue. Degenerate codons that also encoded the wild-type residue were deployed to avoid excessive mutagenesis of target protein. For the same reason, only up to 20 positions were targeted for mutagenesis at a time. These residues were a combination of contact residues and non-contact interface residues.

The oligonucleotides were dissolved in sterile water, mixed in equimolar ratios, heated to 95° C. for five minutes and slowly cooled to room temperature for annealing. ECD-specific oligonucleotide primers that anneal to the start and end of the ECDs, respectively, were then used to generate PCR product. The ECD-specific oligonucleotides which overlap by 40-50 bp with a modified version of pBYDS03 cloning vector (Life Technologies USA), beyond and including the BamH1 and Kpn1 cloning sites, were then used to amplify 100 ng of PCR product from the prior step to generate a total of 5 μg of DNA. Both PCR's were by polymerase chain reaction (PCR) using OneTaq 2×PCR master mix (New England Biolabs, USA). The second PCR products were purified using a PCR purification kit (Qiagen, Germany) and resuspended in sterile deionized water. To prepare for library insertion, a modified yeast display version of vector pBYDS03 was digested with BamH1 and Kpn1 restriction enzymes (New England Biolabs, USA) and the large vector fragment was gel-purified and dissolved in sterile, deionized water. Electroporation-ready DNA for the next step was generated by mixing 12 μg of library DNA with 4 μg of linearized vector in a total volume of 50 μl deionized and sterile water. An alternative way to generate targeted libraries was to carry out site-directed mutagenesis (Multisite kit, Agilent, USA) of target ECDs with oligonucleotides containing degenerate codons. This approach was used to generate sublibraries that only target specific stretches of the DNA for mutagenesis. In these cases, sublibraries were mixed before proceeding to the selection steps. In general, library sizes were in the range of 10E7 to 10E8 clones, except that sublibraries were only in the range of 10E4 to 10E5. Large libraries and sublibraries were generated for CD80, ICOSL, CD86 and NKp30. Sublibraries were generated for CD80, ICOSL and NKp30.

B. Random Libraries

Random libraries were also constructed to identify variants of the ECD of CD80 (SEQ ID NO:28), CD86 (SEQ ID NO: 29), ICOSL (SEQ ID NO:32) and NKp30 (SEQ ID NO:54. DNA encoding wild-type ECDs was cloned between the BamH1 and Kpn1 sites of modified yeast display vector pBYDS03 and then released using the same restriction enzymes. The released DNA was then mutagenized with the Genemorph II kit (Agilent, USA) so as to generate an average of three to five amino acid changes per library variant. Mutagenized DNA was then amplified by the two-step PCR and further processed as described above for targeted libraries.

### Example 2

### Introduction of DNA Libraries into Yeast

Example 2 describes the introduction of CD80, CD86, ICOSL and NKp30 DNA libraries into yeast.

To introduce degenerate and random library DNA into yeast, electroporation-competent cells of yeast strain BJ5464 (ATCC.org; ATCC number 208288) were prepared and electroporated on a Gene Pulser II (Biorad, USA) with the electroporation-ready DNA from the step above essentially as described (Colby, D. W. et al. 2004 Methods Enzymology 388, 348-358). The only exception is that transformed cells were grown in non-inducing minimal selective SCD-Leu medium to accommodate the LEU2 selective marker carried by modified plasmid pBYDS03.

Library size was determined by plating dilutions of freshly recovered cells on SCD-Leu agar plates and then extrapolating library size from the number of single colonies from plating that generated at least 50 colonies per plate. The remainder of the electroporated culture was grown to saturation and cells from this culture were subcultured into the same medium once more to minimize the fraction of untransformed cells. To maintain library diversity, this subculturing step was carried out using an inoculum that contained at least 10× more cells than the calculated library size. Cells from the second saturated culture were resuspended in fresh medium containing sterile 25% (weight/volume) glycerol to a density of 10E10/ml and frozen and stored at −80° C. (frozen library stock).

One liter of SCD-Leu media consists of 14.7 grams of sodium citrate, 4.29 grams of citric acid monohydrate, 20 grams of dextrose, 6.7 grams of Difco brand yeast nitrogen base, and 1.6 grams yeast synthetic drop-out media supplement without leucine. Media was filtered sterilized before use using a 0.2 μM vacuum filter device.

Library size was determined by plating dilutions of freshly recovered cells on SCD-Leu agar plates and then extrapolating library size from the number of single colonies from a plating that generate at least 50 colonies per plate.

To segregate plasmid from cells that contain two or more different library clones, a number of cells corresponding to 10 times the library size, were taken from the overnight SCD-Leu culture and subcultured 1/100 into fresh SCD-Leu medium and grown overnight. Cells from this overnight culture were resuspended in sterile 25% (weight/volume) glycerol to a density of 10E10/ml and frozen and stored at −80° C. (frozen library stock).

### Example 3

### Yeast Selection

Example 3 describes the selection of yeast expressing affinity modified variants of CD80, CD86, ICOSL and NKp30.

A number of cells equal to at least 10 times the library size were thawed from individual library stocks, suspended to 0.1×10E6 cells/ml in non-inducing SCD-Leu medium, and grown overnight. The next day, a number of cells equal to 10 times the library size were centrifuged at 2000 RPM for two minutes and resuspended to 0.5×10E6 cells/ml in inducing SCDG-Leu media. One liter of the SCDG-Leu induction media consists of 5.4 grams Na₂HPO₄, 8.56 grams of NaH₂PO₄*H₂O, 20 grams galactose, 2.0 grams dextrose, 6.7 grams Difco yeast nitrogen base, and 1.6 grams of yeast synthetic drop out media supplement without leucine dissolved in water and sterilized through a 0.22 μm membrane filter device. The culture was grown for two days at 20° C. to induce expression of library proteins on the yeast cell surface.

Cells were processed with magnetic beads to reduce non-binders and enrich for all CD80, CD86, ICOSL or NKp30 variants with the ability to bind their exogenous recombinant counter-structure proteins (cognate binding partners). For example, yeast displayed targeted or random CD80 libraries were selected against each of CD28, CTLA-4, PD-L1. ICOSL libraries were selected against ICOS and CD28 and NKp30 libraries were selected against B7-H6. This was then followed by two to three rounds of fluorescence activated cell sorting (FACS) using exogenous counter-structure protein staining to enrich the fraction of yeast cells that displays improved binders. Magnetic bead enrichment and selections by flow cytometry are essentially as described in Keith D. Miller, 1 Noah B. Pefaur, 2 and Cheryl L. Baird1 Current Protocols in Cytometry 4.7.1-4.7.30, July 2008.

With CD80, CD86, ICOSL, and NKp30 libraries, target ligand proteins were sourced from R&D Systems (USA) as follows: human rCD28.Fc (i.e., recombinant CD28-Fc fusion protein), rPDL1.Fc, rCTLA4.Fc, rICOS.Fc, and rB7H6.Fc. Magnetic streptavidin beads were obtained from New England Biolabs, USA. For biotinylation of counter-structure protein, biotinylation kit cat#21955, Life Technologies, USA, was used. For two-color, flow cytometric sorting, a Becton Dickinson FACS Aria II sorter was used. CD80, CD86, ICOSL, or NKp30 display levels were monitored with an anti-hemagglutinin tag antibody labeled with Alexafluor 488 (Life Technologies, USA). Ligand binding Fc fusion proteins rCD28.Fc, rCTLA4.Fc, rPDL1.Fc, rICOS.Fc, or rB7-H6.Fc were detected with PE conjugated human Ig specific goat Fab (Jackson ImmunoResearch, USA). Doublet yeast were gated out using forward scatter (FSC)/side scatter (SSC) parameters, and sort gates were based upon higher ligand binding detected in FL2 that possessed more limited HA tag expression binding in FL1.

Yeast outputs from the flow cytometric sorts were assayed for higher specific binding affinity. Sort output yeast were expanded and re-induced to express the particular IgSF affinity modified domain variants they encode. This population then can be compared to the parental, wild-type yeast strain, or any other selected outputs, such as the bead output yeast population, by flow cytometry.

For ICOSL, the second sort outputs (F2) were compared to parental ICOSL yeast for binding of each rICOS.Fc, rCD28.Fc, and rCTLA4.Fc by double staining each population with anti-HA (hemagglutinin) tag expression and the anti-human Fc secondary to detect ligand binding.

In the case of ICOSL yeast variants selected for binding to ICOS, the F2 sort outputs gave Mean Fluorescence Intensity (MFI) values of 997, when stained with 5.6 nM rICOS.Fc, whereas the parental ICOSL strain MFI was measured at 397 when stained with the same concentration of rICOS.Fc. This represents a roughly three-fold improvement of the average binding in this F2 selected pool of clones, and it is predicted that individual clones from that pool will have much better improved MFI/affinity when individually tested.

In the case of ICOSL yeast variants selected for binding to CD28, the F2 sort outputs gave MFI values of 640 when stained with 100 nM rCD28.Fc, whereas the parental ICOSL strain MFI was measured at 29 when stained with the same concentration of rCD28.Fc (22-fold improvement). In the case of ICOSL yeast variants selected for binding to CTLA4, the F2 sort outputs gave MFI values of 949 when stained with 100 nM rCTLA4.Fc, whereas the parental ICOSL strain MFI was measured at 29 when stained with the same concentration of rCTLA4.Fc (32-fold improvement).

In the case of NKp30 yeast variants selected for binding to B7-H6, the F2 sort outputs gave MFI values of 533 when stained with 16.6 nM rB7H6.Fc, whereas the parental NKp30 strain MFI was measured at 90 when stained with the same concentration of rB7H6.Fc (6-fold improvement).

Importantly, the MFIs of all F2 outputs described above when measured with the anti-HA tag antibody on FL1 did not increase and sometimes decreased compared to wild-type strains, indicating that increased binding was not a function of increased expression of the selected variants on the surface of yeast, and validated gating strategies of only selecting mid to low expressors with high ligand binding.

### Example 4 Reformatting Selection Outputs as Fc-Fusions and in Various Immunomodulatory Protein Types

Example 4 describes reformatting of selection outputs as immunomodulatory proteins containing an affinity modified (variant) extracellular domain (ECD) of CD80 or ICOSL fused to an Fc molecule (variant ECD-Fc fusion molecules).

Output cells from final flow cytometric CD80 and ICOSL sorts were grown to terminal density in SCD-Leu medium. Plasmid DNA from each output were isolated using a yeast plasmid DNA isolation kit (Zymoresearch, USA). For Fc fusions, PCR primers with added restriction sites suitable for cloning into the Fc fusion vector of choice were used to batch-amplify from the plasmid DNA preps the coding DNA for the mutant target's ECD. After restriction digestion, the PCR products were ligated into an appropriate Fc fusion vector followed by chemical transformation into strain XL1 Blue E. Coli (Agilent, USA) or NEB5alpha (New England Biolabs) as directed by supplierExemplary of an Fc fusion vector is pFUSE-hIgG1-Fc2 (Invivogen, USA).

Dilutions of transformation reactions were plated on LB-agar containing 100 μg/ml carbenicillin (Teknova, USA) to generate single colonies. Up to 96 colonies from each transformation were then grown in 96 well plates to saturation overnight at 37° C. in LB-broth (Teknova cat # L8112) and a small aliquot from each well was submitted for DNA sequencing of the ECD insert in order to identify the mutation(s) in all clones. Sample preparation for DNA sequencing was carried out using protocols provided by the service provider (Genewiz; South Plainfield, N.J.). After removal of sample for DNA sequencing, glycerol was then added to the remaining cultures for a final glycerol content of 25% and plates were stored at −20° C. for future use as master plates (see below). Alternatively, samples for DNA sequencing were generated by replica plating from grown liquid cultures to solid agar plates using a disposable 96 well replicator (VWR, USA). These plates were incubated overnight to generate growth patches and the plates were submitted to Genewiz as specified by Genewiz.

After identification of clones of interest from analysis of Genewiz-generated DNA sequencing data, clones of interest were recovered from master plates and individually grown to density in 5 ml liquid LB-broth containing 100 μg/ml carbenicillin (Teknova, USA) and 2 ml of each culture were then used for preparation of approximately 10 μg of miniprep plasmid DNA of each clone using a standard kit such as the Pureyield kit (Promega). Identification of clones of interest generally involved the following steps. First, DNA sequence data files were downloaded from the Genewiz website. All sequences were then manually curated so that they start at the beginning of the ECD coding region. The curated sequences were then batch-translated using a suitable program available at the URL: www.ebi.ac.uk/Tools/st/emboss_transeq/. The translated sequences were then aligned using a suitable program available at the URL:multalin.toulouse.inra.fr/multalin/multalin.html.

Clones of interest were then identified using the following criteria: 1.) identical clone occurs at least two times in the alignment and 2.) a mutation occurs at least two times in the alignment and preferably in distinct clones. Clones that meet at least one of these criteria were clones that have been enriched by our sorting process due to improved binding.

The methods generated immunomodulatory proteins containing an ECD of CD80 or ICOSL with at least one affinity-modified domain in which the encoding DNA was generated to encode a protein designed as follows: signal peptide followed by variant (mutant) ECD followed by a linker of three alanines (AAA) followed by a human IgG1 Fc containing the mutation N297G (N82G with reference to wild-type human IgG1 Fc set forth in SEQ ID NO: 226). The human IgG1 Fc also contained the mutations R292C and V302C (corresponding to R77C and V87C with reference to wild-type human IgG1 Fc set forth in SEQ ID NO: 226). Since the construct does not include any antibody light chains that can form a covalent bond with a cysteine, the human IgG1 Fc also contained replacement of the cysteine residues to a serine residue at position 5 (C5S) compared to the wild-type or unmodified Fc set forth in SEQ ID NO: 226.

In addition, Example 8 below describes further immunomodulatory proteins that were generated as stack constructs containing at least two different affinity modified domains from identified variant CD80, CD86, ICOSL, and NKp30 molecules linked together and fused to an Fc.

### Example 5

### Expression and Purification of Fc-Fusions

Example 5 describes the high throughput expression and purification of Fc-fusion proteins containing variant ECD CD80, CD86, ICOSL, and Nkp30.

Recombinant variant Fc fusion proteins were produced with Expi293 expression system (Invitrogen, USA). 4 μg of each plasmid DNA from the previous step was added to 200 μl Opti-MEM (Invitrogen, USA) at the same time as 10.8 μl ExpiFectamine was separately added to another 200 μl Opti-MEM. After 5 minutes, the 200 μl of plasmid DNA was mixed with the 200 μl of ExpiFectamine and was further incubated for an additional 20 minutes before adding this mixture to cells. Ten million Expi293 cells were dispensed into separate wells of a sterile 10 ml, conical bottom, deep 24 well growth plate (Thomson Instrument Company, USA) in a volume 3.4 ml Expi293 media (Invitrogen, USA). Plates were shaken for 5 days at 120 RPM in a mammalian cell culture incubator set to 95% humidity and 8% CO₂. Following a 5 day incubation, cells were pelleted and culture supernatants were removed.

Protein was purified from supernatants using a high throughput 96 well Protein A purification kit using the manufacturer's protocol (Catalog number 45202, Life Technologies, USA). Resulting elution fractions were buffer exchanged into PBS using Zeba 96 well spin desalting plate (Catalog number 89807, Life Technologies, USA) using the manufacturer's protocol. Purified protein was quantitated using 280 nm absorbance measured by Nanodrop instrument (Thermo Fisher Scientific, USA), and protein purity was assessed by loading 5 μg of protein on NUPAGE pre-cast, polyacrylamide gels (Life Technologies, USA) under denaturing and reducing conditions and subsequent gel electrophoresis. Proteins were visualized in gel using standard Coomassie staining.

### Example 6

### Assessment of Binding and Activity of Affinity-Matured IgSF Domain-Containing Molecules

A. Binding to Cell Surface-Expressed Counter Structures

This Example describes Fc-fusion binding studies to show specificity and affinity of CD80 and ICOSL domain variant immunomodulatory proteins for cognate binding partners.

To produce cells expressing cognate binding partners, full-length mammalian surface expression constructs for each of human CD28, CTLA4, PD-L1, ICOS and B7-H6 were designed in pcDNA3.1 expression vector (Life Technologies) and sourced from Genscript, USA. Binding studies were carried out using the Expi293F transient transfection system (Life Technologies, USA) described above. The number of cells needed for the experiment was determined, and the appropriate 30 ml scale of transfection was performed using the manufacturer's suggested protocol. For each CD28, CTLA-4, PD-L1, ICOS, B7-H6, or mock 30 ml transfection, 75 million Expi293F cells were incubated with 30 μg expression construct DNA and 1.5 ml diluted ExpiFectamine 293 reagent for 48 hours, at which point cells were harvested for staining.

For staining by flow cytometry, 200,000 cells of appropriate transient transfection or negative control were plated in 96 well round bottom plates. Cells were spun down and resuspended in staining buffer (PBS (phosphate buffered saline), 1% BSA (bovine serum albumin), and 0.1% sodium azide) for 20 minutes to block non-specific binding. Afterwards, cells were centrifuged again and resuspended in staining buffer containing 100 nM to 1 nM variant immunomodulatory protein, depending on the experiment of each candidate CD80 variant Fc, ICOSL variant Fc, or stacked IgSF variant Fc fusion protein in 50 μl. Primary staining was performed on ice for 45 minutes, before washing cells in staining buffer twice. PE-conjugated anti-human Fc (Jackson ImmunoResearch, USA) was diluted 1:150 in 50 μl staining buffer and added to cells and incubated another 30 minutes on ice. Secondary antibody was washed out twice, cells were fixed in 4% formaldehyde/PBS, and samples were analyzed on FACScan flow cytometer (Becton Dickinson, USA).

Mean Fluorescence Intensity (MFI) was calculated for each transfectant and negative parental line with Cell Quest Pro software (Becton Dickinson, USA).

B. Bioactivity Characterization

This Example further describes Fc-fusion variant protein bioactivity characterization in human primary T cell in vitro assays.

1. Mixed Lymphocyte Reaction (MLR)

Soluble rICOSL.Fc or rCD80.Fc bioactivity was tested in a human Mixed Lymphocyte Reaction (MLR). Human primary dendritic cells (DC) were generated by culturing monocytes isolated from PBMC (BenTech Bio, USA) in vitro for 7 days with 500 U/ml rIL-4 (R&D Systems, USA) and 250 U/ml rGM-CSF (R&D Systems, USA) in Ex-Vivo 15 media (Lonza, Switzerland). 10,000 matured DC and 100,000 purified allogeneic CD4+ T cells (BenTech Bio, USA) were co-cultured with ICOSL variant fusion protein, CD80 variant Fc fusion protein, or controls in 96 well round bottom plates in 200 μL final volume of Ex-Vivo 15 media. On day 5, IFN-gamma secretion in culture supernatants was analyzed using the Human IFN-gamma Duoset ELISA kit (R&D Systems, USA). Optical density was measured by VMax ELISA Microplate Reader (Molecular Devices, USA) and quantitated against titrated rIFN-gamma standard included in the IFN-gamma Duo-set kit (R&D Systems, USA).

2. Anti-CD3 Coimmobilization Assay

Costimulatory bioactivity of ICOSL fusion variants and CD80 Fc fusion variants was determined in anti-CD3 coimmobilization assays. 1 nM or 4 nM mouse anti-human CD3 (OKT3, Biolegends, USA) was diluted in PBS with 1 nM to 80 nM rICOSL.Fc or rCD80.Fc variant proteins. This mixture was added to tissue culture treated flat bottom 96 well plates (Corning, USA) overnight to facilitate adherence of the stimulatory proteins to the wells of the plate. The next day, unbound protein was washed off the plates and 100,000 purified human pan T cells (BenTech Bio, US) or human T cell clone BC3 (Astarte Biologics, USA) were added to each well in a final volume of 200 μl of Ex-Vivo 15 media (Lonza, Switzerland). Cells were cultured 3 days before harvesting culture supernatants and measuring human IFN-gamma levels with Duoset ELISA kit (R&D Systems, USA) as mentioned above.

C. Results

Results for the binding and activity studies for exemplary tested variants are shown in Tables 8-10. In particular, Table 8 indicates exemplary IgSF domain amino acid substitutions (replacements) in the ECD of CD80 selected in the screen for affinity-maturation against the respective cognate structure CD28. Table 9 indicates exemplary IgSF domain amino acid substitutions (replacements) in the ECD of CD80 selected in the screen for affinity-maturation against the respective cognate structure PD-L1. Table 10 indicates exemplary IgSF domain amino acid substitutions (replacements) in the ECD of ICOSL selected in the screen for affinity-maturation against the respective cognate structures ICOS and CD28. For each Table, the exemplary amino acid substitutions are designated by amino acid position number corresponding to the respective reference unmodified ECD sequence as follows. For example, the reference unmodified ECD sequence in Tables 8 and 9 is the unmodified CD80 ECD sequence set forth in SEQ ID NO:28 and the reference unmodified ECD sequence in Table 10 is the unmodified ICOSL ECD sequence (SEQ ID NO:32). The amino acid position is indicated in the middle, with the corresponding unmodified (e.g. wild-type) amino acid listed before the number and the identified variant amino acid substitution listed after the number. Column 2 sets forth the SEQ ID NO identifier for the variant ECD for each variant ECD-Fc fusion molecule.

Also shown is the binding activity as measured by the Mean Fluorescence Intensity (MFI) value for binding of each variant Fc-fusion molecule to cells engineered to express the cognate counter structure ligand and the ratio of the MFI compared to the binding of the corresponding unmodified ECD-Fc fusion molecule not containing the amino acid substitution(s) to the same cell-expressed counter structure ligand. The functional activity of the variant Fc-fusion molecules to modulate the activity of T cells also is shown based on the calculated levels of IFN-gamma in culture supernatants (pg/ml) generated either i) with the indicated variant ECD-Fc fusion molecule coimmoblized with anti-CD3 or ii) with the indicated variant ECD-Fc fusion molecule in an MLR assay. The Tables also depict the ratio of IFN-gamma produced by each variant ECD-Fc compared to the corresponding unmodified ECD-Fc in both functional assays.

As shown, the selections resulted in the identification of a number of CD80 or ICOSL IgSF domain variants that were affinity-modified to exhibit increased binding for at least one, and in some cases more than one, cognate counter structure ligand. In addition, the results showed that affinity modification of the variant molecules also exhibited improved activities to both increase and decrease immunological activity depending on the format of the molecule. For example, coimmobilization of the ligand likely provides a multivalent interaction with the cell to cluster or increase the avidity to favor agonist activity and increase T cell activation compared to the unmodified (e.g. wildtype) ECD-Fc molecule not containing the amino acid replacement(s). However, when the molecule is provided as a bivalent Fc molecule in solution, the same IgSF domain variants exhibited an antagonist activity to decrease T cell activation compared to the unmodified (e.g. wildtype) ECD-Fv molecule not containing the amino acid replacement(s).

TABLE 8
CD80 variants selected against CD28. Molecule sequences, binding data, and
costimulatory bioactivity data.

| | | | | | Coimmobilization | |
|---|---|---|---|---|---|---|
| | | | | | with | |
| | | Binding | | | anti-CD3 | MLR |
| | | CD28 | CTLA-4 | PD-L1 | IFN-gamma | IFN-gamma |
| | SEQ ID | MFI | MFI | MFI | pg/ml | levels pg/ml |
| | NO | (parental | (parental | (parental | (parental | (parental |
| CD80 mutation(s) | (ECD) | ratio) | ratio) | ratio) | ratio) | ratio) |
| L70Q/A91G | 55 | 125 | 283 | 6 | 93 | 716 |
| | | (1.31) | (1.36) | (0.08) | (1.12) | (0.83) |
| L70Q/A91G/T130A | 56 | 96 | 234 | 7 | 99 | 752 |
| | | (1.01) | (1.13) | (0.10) | (1.19) | (0.87) |
| L70Q/A91G/I118A/ | 57 | 123 | 226 | 7 | 86 | 741 |
| T120S/T130A | | (1.29) | (1.09) | (0.10) | (1.03) | (0.86) |
| V4M/L70Q/A91G/ | 58 | 89 | 263 | 6 | 139 | 991 |
| T120S/T130A | | (0.94) | (1.26) | (0.09) | (1.67) | (1.14) |
| L70Q/A91G/T120S/ | 59 | 106 | 263 | 6 | 104 | 741 |
| T130A | | (1.12) | (1.26) | (0.09) | (1.25) | (0.86) |
| V20L/L70Q/A91S/ | 60 | 105 | 200 | 9 | 195 | 710 |
| T120S/T130A | | (1.11) | (0.96) | (0.13) | (2.34) | (0.82) |
| S44P/L70Q/A91G/ | 61 | 88 | 134 | 5 | 142 | 854 |
| T130A | | (0.92) | (0.64) | (0.07) | (1.71) | (0.99) |
| L70Q/A91G/E117G/ | 62 | 120 | 193 | 6 | 98 | 736 |
| T120S/T130A | | (1.27) | (0.93) | (0.08) | (1.05) | (0.85) |
| A91G/T120S/ | 63 | 84 | 231 | 44 | 276 | 714 |
| T130A | | (0.89) | (1.11) | (0.62) | (3.33) | (0.82) |
| L70R/A91G/T120S/ | 64 | 125 | 227 | 6 | 105 | 702 |
| T130A | | (1.32) | (1.09) | (0.09) | (1.26) | (0.81) |
| L70Q/E81A/A91G/ | 65 | 140 | 185 | 18 | 98 | 772 |
| T120S/I127T/ | | (1.48) | (0.89) | (0.25) | (1.18) | (0.89) |
| T130A | | | | | | |
| L70Q/Y87N/A91G/ | 66 | 108 | 181 | 6 | 136 | 769 |
| T130A | | (1.13) | (0.87) | (0.08) | (1.63) | (0.89) |
| T28S/L70Q/A91G/ | 67 | 32 | 65 | 6 | 120 | 834 |
| E95K/T120S/T130A | | (0.34) | (0.31) | (0.08) | (1.44) | (0.96) |
| N63S/L70Q/A91G/ | 68 | 124 | 165 | 6 | 116 | 705 |
| T120S/T130A | | (1.30) | (0.79) | (0.08) | (1.39) | (0.81) |
| K36E/I67T/L70Q/ | 69 | 8 | 21 | 5 | 53 | 852 |
| A91G/T120S/ | | (0.09) | (0.10) | (0.08) | (0.63) | (0.98) |
| T130A/N152T | | | | | | |
| E52G/L70Q/A91G/ | 70 | 113 | 245 | 6 | 94 | 874 |
| T120S/T130A | | (1.19) | (1.18) | (0.08) | (1.13) | (1.01) |
| K37E/F59S/L70Q/ | 71 | 20 | 74 | 6 | 109 | 863 |
| A91G/T120S/ | | (0.21) | (0.36) | (0.08) | (1.31) | (1.00) |
| T130A | | | | | | |
| A91G/S103P | 72 | 39 | 56 | 9 | 124 | 670 |
| | | (0.41) | (0.27) | (0.13) | (1.49) | (0.77) |
| K89E/T130A | 73 | 90 | 148 | 75 | 204 | 761 |
| | | (0.95) | (0.71) | (1.07) | (2.45) | (0.88) |
| A91G | 74 | 96 | 200 | 85 | 220 | 877 |
| | | (1.01) | (0.96) | (1.21) | (2.65) | (1.01) |
| D60V/A91G/T120S/ | 75 | 111 | 222 | 12 | 120 | 744 |
| T130A | | (1.17) | (1.07) | (0.18) | (1.44) | (0.86) |
| K54M/A91G/T120S | 76 | 68 | 131 | 5 | 152 | 685 |
| | | (0.71) | (0.63) | (0.08) | (1.83) | (0.79) |
| M38T/L70Q/E77G/ | 77 | 61 | 102 | 5 | 119 | 796 |
| A91G/T120S/ | | (0.64) | (0.49) | (0.07) | (1.43) | (0.92) |
| T130A/N152T | | | | | | |
| R29H/E52G/L70R/ | 78 | 100 | 119 | 5 | 200 | 740 |
| E88G/A91G/T130A | | (1.05) | (0.57) | (0.08) | (2.41) | (0.85) |
| Y31H/T41G/L70Q/ | 79 | 85 | 85 | 6 | 288 | 782 |
| A91G/T120S/ | | (0.89) | (0.41) | (0.08) | (3.47) | (0.90) |
| T130A | | | | | | |
| V68A/110A | 80 | 103 | 233 | 48 | 163 | 861 |
| | | (1.08) | (1.12) | (0.68) | (1.96) | (0.99) |
| S66H/D90G/T110A/ | 81 | 33 | 121 | 11 | 129 | 758 |
| F116L | | (0.35) | (0.58) | (0.15) | (1.55) | (0.88) |
| R29H/E52G/T120S/ | 82 | 66 | 141 | 11 | 124 | 800 |
| T130A | | (0.69) | (0.68) | (0.15) | (1.49) | (0.92) |
| A91G/L102S | 83 | 6 | 6 | 5 | 75 | 698 |
| | | (0.06) | (0.03) | (0.08) | (0.90) | (0.81) |
| I67T/L70Q/A91G/ | 84 | 98 | 160 | 5 | 1751 | 794 |
| T120S | | (1.03) | (0.77) | (0.08) | (21.1) | (0.92) |
| L70Q/A91G/T110A/ | 85 | 8 | 14 | 5 | 77 | 656 |
| T120S/T130A | | (0.09) | (0.07) | (0.07) | (0.93) | (0.76) |
| M38V/T41D/M43I/ | 86 | 5 | 8 | 8 | 82 | 671 |
| W50G/D76G/V83A/ | | (0.06) | (0.04) | (0.11) | (0.99) | (0.78) |
| K89E/T120S/T130A | | | | | | |
| V22A/L70Q/S121P | 87 | 5 | 7 | 5 | 105 | 976 |
| | | (0.06) | (0.04) | (0.07) | (1.27) | (1.13) |
| A12V/S15F/Y31H/ | 88 | 6 | 6 | 5 | 104 | 711 |
| T41G/T130A/P137L/ | | (0.06) | (0.03) | (0.08) | (1.25) | (0.82) |
| N152T | | | | | | |
| I67F/L70R/E88G/ | 89 | 5 | 6 | 6 | 62 | 1003 |
| A91G/T120S/T130A | | (0.05) | (0.03) | (0.08) | (0.74) | (1.16) |
| E24G/L25P/L70Q/ | 90 | 26 | 38 | 8 | 101 | 969 |
| T120S | | (0.27) | (0.18) | (0.11) | (1.21) | (1.12) |
| A91G/F92L/F108L/ | 91 | 50 | 128 | 16 | 59 | 665 |
| T120S | | (0.53) | (0.61) | (0.11) | (0.71) | (0.77) |
| WT CD80 | 28 | 95 | 208 | 70 | 83 | 866 |
| | | (1.00) | (1.00) | (1.00) | (1.00) | (1.00) |

TABLE 9
CD80 variants selected against PD-L1. Molecule sequences, binding data, and
costimulatory bioactivity data.

| | | | | | Coimmobilization | MLR |
|---|---|---|---|---|---|---|
| | | | | | with | IFN- |
| | | Binding | | | anti-CD3 | gamma |
| | | CD28 | CTLA-4 | PD-L1 | IFN-gamma | levels |
| | SEQ ID | MFI | MFI | MFI | pg/ml | pg/ml |
| | NO | (parental | (parental | (parental | (parental | (parental |
| CD80 mutation(s) | (ECD) | ratio) | ratio) | ratio) | ratio) | ratio) |
| R29D/Y31L/Q33H/ | 92 | 1071 | 1089 | 37245 | 387 | 5028 |
| K36G/M38I/T41A/ | | (0.08) | (0.02) | (2.09) | (0.76) | (0.26) |
| M43R/M47T/E81V/ | | | | | | |
| L85R/K89N/A91T/ | | | | | | |
| F92P/K93V/R94L/ | | | | | | |
| I118T/N149S | | | | | | |
| R29D/Y31L/Q33H/ | 93 | 1065 | 956 | 30713 | 400 | 7943 |
| K36G/M38I/T41A/ | | (0.08) | (0.02) | (1.72) | (0.79) | (0.41) |
| M43R/M47T/E81V/ | | | | | | |
| L85R/K89N/A91T/ | | | | | | |
| F92P/K93V/R94L/ | | | | | | |
| N144S/N149S | | | | | | |
| R29D/Y31L/Q33H/ | 94 | 926 | 954 | 47072 | 464 | 17387 |
| K36G/M38I/T41A/ | | (0.07) | (0.02) | (2.64) | (0.91) | (0.91) |
| M42T/M43R/M47T/ | | | | | | |
| E81V/L85R/K89N/ | | | | | | |
| A91T/F92P/K93V/ | | | | | | |
| R94L/L148S/N149S | | | | | | |
| E24G/R29D/Y31L/ | 95 | 1074 | 1022 | 1121 | 406 | 13146 |
| Q33H/K36G/M38I/ | | (0.08) | (0.02) | (0.06) | (0.80) | (0.69) |
| T41A/M43R/M47T/ | | | | | | |
| F59L/E81V/L85R/ | | | | | | |
| K89N/A91T/F92P/ | | | | | | |
| K93V/R94L/H96R | | | | | | |
| R29D/Y31L/Q33H/ | 96 | 1018 | 974 | 25434 | 405 | 24029 |
| K36G/M38I/T41A/ | | (0.08) | (0.02) | (1.43) | (0.80) | (1.25) |
| M43R/M47T/E81V/ | | | | | | |
| L85R/K89N/A91T/ | | | | | | |
| F92P/K93V/R94L/ | | | | | | |
| N149S | | | | | | |
| R29V/M43Q/E81R/ | 97 | 1029 | 996 | 1575 | 342 | 11695 |
| L85I/K89R/D90L/ | | (0.08) | (0.02) | (0.09) | (0.67) | (0.61) |
| A91E/F92N/K93Q/ | | | | | | |
| R94G | | | | | | |
| T41I/A91G | 98 | 17890 | 50624 | 12562 | 433 | 26052 |
| | | (1.35) | (1.01) | (0.70) | (0.85) | (1.36) |
| K89R/D90K/A91G/ | 99 | 41687 | 49429 | 20140 | 773 | 6345 |
| F92Y/K93R/N122S/ | | (3.15) | (0.99) | (1.13) | (1.52) | (0.33) |
| N178S | | | | | | |
| K89R/D90K/A91G/ | 100 | 51663 | 72214 | 26405 | 1125 | 9356 |
| F92Y/K93R | | (3.91) | (1.44) | (1.48) | (2.21) | (0.49) |
| K36G/K37Q/M38I/ | 101 | 1298 | 1271 | 3126 | 507 | 3095 |
| F59L/E81V/L85R/ | | (0.10) | (0.03) | (0.18) | (1.00) | (0.16) |
| K89N/A91T/F92P/ | | | | | | |
| K93V/R94L/E99G/ | | | | | | |
| T130A/N149S | | | | | | |
| AE88D/K89R/D90K/ | 102 | 31535 | 50868 | 29077 | 944 | 5922 |
| A91G/F92Y/K93R | | (2.38) | (1.02) | (1.63) | (1.85) | (0.31) |
| K36G/K37Q/M38I/ | 103 | 1170 | 1405 | 959 | 427 | 811 |
| L40M | | (0.09) | (0.03) | (0.05) | (0.84) | (0.04) |
| K36G | 104 | 29766 | 58889 | 20143 | 699 | 30558 |
| | | (2.25) | (1.18) | (1.13) | (1.37) | (1.59) |
| WTCD80 | 28 | 13224 | 50101 | 17846 | 509 | 19211 |
| | | (1.00) | (1.00) | (1.00) | (1.00) | (1.00) |

TABLE 10
ICOSL variants selected against CD28 or ICOS. Molecule sequences,
binding data, and costimulatory bioactivity data.

| | | | | | | Coimmobilization | | | |
|---|---|---|---|---|---|---|---|---|---|
| | | | | | | with anti-CD3 | | MLR | |
| | | Binding | | | | IFN-gamma | | IFN-gamma | |
| | SEQ | ICOS OD | | CD28 MFI | | pg/ml | | levels pg/ml | |
| ICOSL | ID NO | (parental | | (parental | | (parental | | (parental | |
| mutation(s) | (ECD) | ratio) | | ratio) | | ratio) | | ratio) | |
| N52S | 109 | 1.33 | (1.55) | 162 | (9.00) | 1334 | (1.93) | 300 | (0.44) |
| N52H | 110 | 1.30 | (1.51) | 368 | (20.44) | 1268 | (1.83) | 39 | (0.06) |
| N52D | 111 | 1.59 | (1.85) | 130 | (7.22) | 1943 | (2.80) | 190 | (0.28) |
| N52Y/N57Y/ | 112 | 1.02 | (1.19) | 398 | (22.11) | 510* | (1.47*) | 18 | (0.03) |
| F138L/L203P | | | | | | | | | |
| N52H/N57Y/Q100P | 113 | 1.57 | (1.83) | 447 | (24.83) | 2199 | (3.18) | 25 | (0.04) |
| N52S/Y146C/ | 114 | 1.26 | (1.47) | 39 | (2.17) | 1647 | (2.38) | 152 | (0.22) |
| Y152C | | | | | | | | | |
| N52H/C198R | 115 | 1.16 | (1.35) | 363 | (20.17) | 744* | (2.15*) | ND | (ND) |
| N52H/C140D/T225A | 116 | ND | (ND) | 154 | (8.56) | 522* | (1.51*) | ND | (ND) |
| N52H/C198R/T225A | 117 | 1.41 | (1.64) | 344 | (19.11) | 778* | (2.25*) | 0 | (0) |
| N52H/K92R | 118 | 1.48 | (1.72) | 347 | (19.28) | 288* | (0.83*) | 89 | (0.13) |
| N52H/S99G | 119 | 0.09 | (0.10) | 29 | (1.61) | 184* | (0.53*) | 421 | (0.61) |
| N52Y | 120 | 0.08 | (0.09) | 18 | (1.00) | 184* | (0.53*) | 568 | (0.83) |
| N57Y | 121 | 1.40 | (1.63) | 101 | (5.61) | 580* | (1.68*) | 176 | (0.26) |
| N57Y/Q100P | 122 | 0.62 | (0.72) | 285 | (15.83) | 301* | (0.87*) | 177 | (0.26) |
| N52S/S130G/ | 123 | 0.16 | (0.19) | 24 | (1.33) | 266* | (0.77*) | 1617 | (2.35) |
| Y152C | | | | | | | | | |
| N52S/Y152C | 124 | 0.18 | (0.21) | 29 | (1.61) | 238* | (0.69*) | 363 | (0.53) |
| N52S/C198R | 125 | 1.80 | (2.09) | 82 | (4.56) | 1427 | (2.06) | 201 | (0.29) |
| N52Y/N57Y/Y152C | 126 | 0.08 | (0.09) | 56 | (3.11) | 377* | (1.09*) | 439 | (0.64) |
| N52Y/N57Y/ | 127 | ND | (ND) | 449 | (24.94) | 1192 | (1.72) | ND | (ND) |
| H129P/C198R | | | | | | | | | |
| N52H/L161P/ | 128 | 0.18 | (0.21) | 343 | (19.05) | 643* | (1.86*) | 447 | (0.65) |
| C198R | | | | | | | | | |
| N52S/T113E | 129 | 1.51 | (1.76) | 54 | (3.00) | 451* | (1.30*) | 345 | (0.50) |
| S54A | 130 | 1.62 | (1.88) | 48 | (2.67) | 386* | (1.12*) | 771 | (1.12) |
| N52D/S54P | 131 | 1.50 | (1.74) | 38 | (2.11) | 476* | (1.38*) | 227 | (0.33) |
| N52K/L208P | 132 | 1.91 | (2.22) | 291 | (16.17) | 1509 | (2.18) | 137 | (0.20) |
| N52S/Y152H | 133 | 0.85 | (0.99) | 68 | (3.78) | 2158 | (3.12) | 221 | (0.32) |
| N52D/V151A | 134 | 0.90 | (1.05) | 19 | (1.06) | 341* | (0.99*) | 450 | (0.66) |
| N52H/I143T | 135 | 1.83 | (2.13) | 350 | (19.44) | 2216 | (3.20) | 112 | (0.16) |
| N52S/L80P | 136 | 0.09 | (0.10) | 22 | (1.22) | 192* | (0.55*) | 340 | (0.49) |
| F120S/Y152H/N201S | 137 | 0.63 | (0.73) | 16 | (0.89) | 351* | (1.01*) | 712 | (1.04) |
| N52S/R75Q/L203P | 138 | 1.71 | (1.99) | 12 | (0.67) | 1996 | (2.88) | 136 | (0.20) |
| N52S/D158G | 139 | 1.33 | (1.55) | 39 | (2.17) | 325* | (0.94*) | 277 | (0.40) |
| N52D/Q133H | 140 | 1.53 | (1.78) | 104 | (5.78) | 365* | (1.05*) | 178 | (0.26) |
| WT ICOSL | 32 | 0.86 | (1.00) | 18 | (1.00) | 692/346* | (1.00) | 687 | (1.00) |

*Parental ratio calculated using 346 pg/ml IFN-gamma for WT ICOSL

### Example 7

### Ligand Binding Competition Assay

As shown in Example 6, several CD80 variant molecules exhibited improved binding to one or both of CD28 and PD-L1. To further assess the binding activity of CD80 to ligands CD28 and PD-L1, this Example describes a ligand competition assay assessing the non-competitive nature of exemplary CD80 variants to bind both CD28 and PD-L1.

An ELISA based binding assay was set up incorporating plate-bound CD80 variant A91G ECD-Fc to assess the ability of CD80 to simultaneously bind CD28 and PD-L1. Maxisorp 96 well ELISA plates (Nunc, USA) were coated overnight with 100 nM human recombinant CD80 variant A91G ECD-Fc fusion protein in PBS. The following day unbound protein was washed out, and the plate was blocked with 1% bovine serum albumin (Millipore, USA)/PBS for 1 hour at room temperature. This blocking reagent was washed out three times with PBS/0.05% Tween, which included a two minute incubation on a platform shaker for each wash.

In one arm of the competition assay, CD80 was incubated with CD28, and then CD28-bound CD80 was then assessed for competitive binding in the presence of either the other known CD80 ligand counter structures PD-L1 or CTLA-4 or negative control ligand PD-L2. Specifically, biotinylated recombinant human CD28 Fc fusion protein (rCD28.Fc; R&D Systems) was titrated into the wells starting at 10 nM, diluting out for eight points with 1:2 dilutions in 25 μl volume. Immediately after adding the biotinylated rCD28.Fc, unlabeled competitive binders, recombinant human PD-L1 monomeric his-tagged protein, recombinant human CTLA-4 monomeric his-tagged protein, or a negative control human recombinant PD-L2 Fc fusion protein (R&D Systems) were added to wells at 2000/1000/500 nM respectively in 25 μl volume for a final volume of 50 μl. These proteins were incubated together for one hour before repeating the three wash steps as described above.

After washing, 2.5 ng per well of HRP-conjugated streptavidin (Jackson Immunoresearch, USA) was diluted in 1% BSA/PBS and added to wells to detect bound biotinylated rCD28.Fc. After one hour incubation, wells were washed again three times as described above. To detect signal, 50 μl of TMB substrate (Pierce, USA) was added to wells following wash and incubated for 7 minutes, before adding 50 ul 2M sulfuric acid stop solution. Optical density was determined on an Emax Plus microplate reader (Molecular Devices, USA). Optical density values were graphed in Prism (Graphpad, USA).

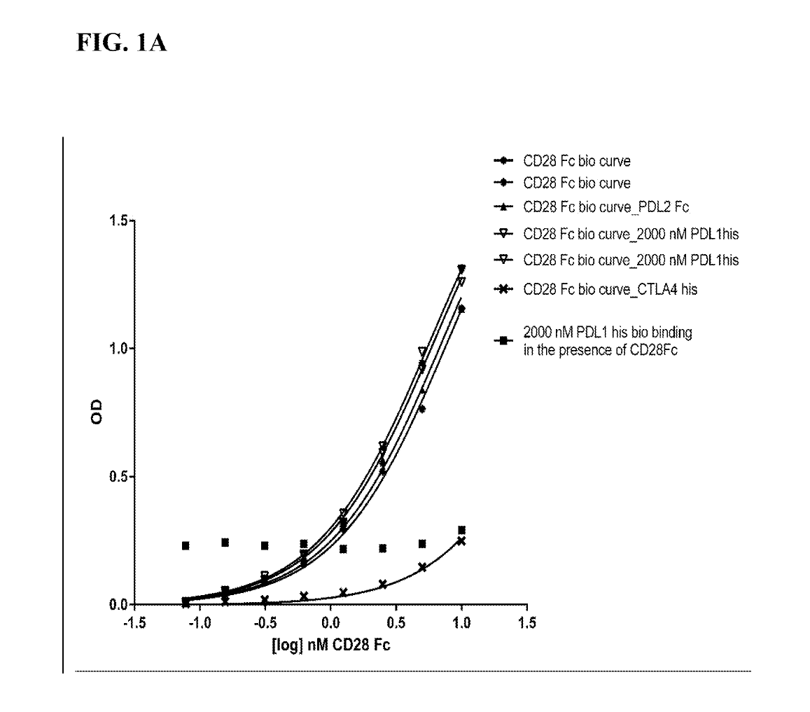

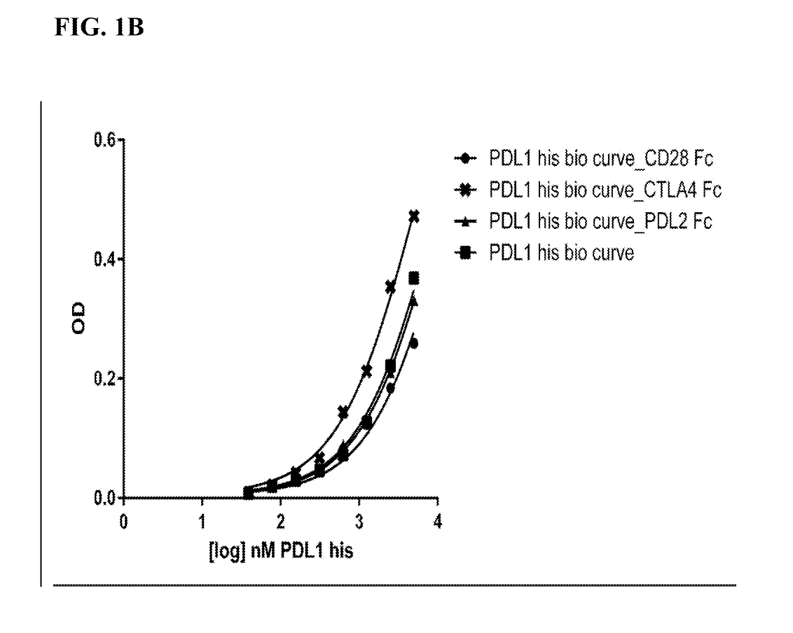

The results are set forth in FIG. 1A. The results showed decreased binding of biotinylated rCD28.Fc to the CD80 variant A91G ECD-Fc fusion protein with titration of the rCD28.Fc. When rCD28.Fc binding was performed in the presence of non-competitive control protein, rPDL2, there was no decrease in CD28 binding for CD80 (solid triangle). In contrast, a competitive control protein, rCTLA-4, when incubated with the CD28.Fc, did result in decreased CD28 binding for CD80 as expected (x line). When recombinant PD-L1 was incubated with CD28.Fc, no decrease in CD28 binding to CD80 was observed, which demonstrated that the epitopes of CD28 and PD-L1 for CD80 are non-competitive. Binding of the recombinant PD-L1 protein used in the CD28 competition assay to CD80 was confirmed by incubating the biotinylated PD-L1 in the presence of non-biotinylated rCD28.Fc (square).

The reverse competition also was set up in which CD80 was incubated with PD-L1, and then PD-L1-bound CD80 was then assessed for competitive binding in the presence of either the other known CD80 ligand counter structures CD28 or CTLA-4 or negative control ligand PD-L2. Specifically, the assay was performed by titrating biotinylated recombinant human PD-L1-his monomeric protein into wells containing the recombinant CD80 variant. Because binding is weaker with this ligand, titrations started at 5000 nM with similar 1:2 dilutions over eight points in 25 μL. When the rPD-L1-his was used to detect binding, the competitive ligands human rCD28.Fc, human rCTLA-4.Fc, or human rPD-L2.Fc control were added at 2.5 nM final concentration in 25 μl for a total volume of 50 μl. The subsequent washes, detection, and OD measurements were the same as described above.

The results are set forth in FIG. 1B. Titrated PD-L1-his binding alone confirmed that PD-L1 bound to the CD80 variant A91G ECD-Fc fusion molecule immobilized on the plate (square). When PD-L1-his binding was performed in the presence of non-competitive control protein, rPDL2, there was no decrease in PD-L1 binding for CD80 (triangle). The CD28-competitive control protein, rCTLA-4, when incubated with the PD-L1-his, did not result in decreased PD-L1 binding for CD80 (x line), even though CTLA-4 is competitive for CD28. This result further demonstrated that lack of competition between CD28 and PD-L1 for CD80 binding. Finally, when PD-L1-his was incubated with CD28.Fc, no decrease in PD-L1 binding to CD80 was observed, which demonstrated that the epitopes of CD28 and PD-L1 for CD80 are non-competitive.

Thus, the results showed that CTLA-4, but not PD-L1 or the negative control PD-L2, competed for binding of CD28 to CD80 (FIG. 1A) and that CD28, CTLA-4, and PD-L2 did not compete for binding of PD-L1 to CD80 (FIG. 1B). Thus, these results demonstrated that CD28 and PD-L1 are non-competitive binders of CD80, and that this non-competitive binding can be demonstrated independently of which ligand is being detected in the ELISA.

### Example 8

### Generation and Assessment of Stacked Molecules Containing Different Affinity-Modified Domains

Selected variant molecules described above that were affinity-modified for one or more counter structure ligand were used to generate “stack” molecule (i.e., Type II immunomodulatory protein) containing two or more affinity-modified IgSF domains. Stack constructs were obtained as geneblocks (Integrated DNA Technologies, Coralville, Iowa) that encode the stack in a format that enables its fusion to Fc by standard Gibson assembly using a Gibson assembly kit (New England Biolabs).

The encoding nucleic acid molecule of all stacks was generated to encode a protein designed as follows: Signal peptide, followed by the first variant IgV of interest, followed by a 15 amino acid linker which is composed of three GGGGS(G4S) motifs (SEQ ID NO:228), followed by the second IgV of interest, followed by two GGGGS linkers (SEQ ID NO: 229) followed by three alanines (AAA), followed by a human IgG1 Fc as described above. To maximize the chance for correct folding of the IgV domains in each stack, the first IgV was preceded by all residues that normally occur in the wild-type protein between this IgV and the signal peptide (leading sequence). Similarly, the first IgV was followed by all residues that normally connect it in the wild-type protein to either the next Ig domain (typically an IgC domain) or if such a second IgV domain is absent, the residues that connect it to the transmembrane domain (trailing sequence). The same design principle was applied to the second IgV domain except that when both IgV domains were derived from same parental protein (e.g. a CD80 IgV stacked with another CD80 IgV), the linker between both was not duplicated.

Table 11 sets forth the design for exemplary stacked constructs. The exemplary stack molecules shown in Table 11 contain the IgV domains as indicated and additionally leading or trailing sequences as described above. In the Table, the following components are present in order: signal peptide (SP; SEQ ID NO:225), IgV domain 1 (IgV1), trailing sequence 1 (TS1), linker 1 (LR1; SEQ ID NO:228), IgV domain 2(IgV2), trailing sequence 2 (TS2), linker 2 (LR2; SEQ ID NO:230) and Fc domain (SEQ ID NO:226 containing C5S/N82G amino acid substitution). In some cases, a leading sequence 1(LS1) is present between the signal peptide and IgV1 and in some cases a leading sequence 2 (LS2) is present between the linker and IgV2.

TABLE 11
Amino acid sequence (SEQ ID NO) of components of exemplary stacked constructs

| | | First domain | | | | Second domain | | | | |
|---|---|---|---|---|---|---|---|---|---|---|
| | SP | LS1 | IgV1 | TS1 | LR1 | LS2 | IgV2 | TS2 | LR2 | Fc |
| NKp30 WT | + | — | SEQ ID | SEQ ID | + | — | SEQ ID | SEQ ID | + | + |
| ICOSL WT | | | NO: 214 | NO: 235 | | | NO: 196 | NO: 233 | | |
| NKp30 | + | — | SEQ ID | SEQ ID | + | — | SEQ ID | SEQ ID | + | + |
| L30V/A60V/S64P/ | | | NO: 215 | NO: 235 | | | NO: 212 | NO: 233 | | |
| S86G | | | | | | | | | | |
| ICOSL | | | | | | | | | | |
| N52S/N57Y/H94D/ | | | | | | | | | | |
| L96F/L98F/Q100R | | | | | | | | | | |
| NKp30 | + | — | SEQ ID | SEQ ID | + | — | SEQ ID | SEQ ID | + | + |
| L30V/A60V/S64P/ | | | NO: 215 | NO: 235 | | | NO: 199 | NO: 233 | | |
| S86G) | | | | | | | | | | |
| ICOSL N52D | | | | | | | | | | |
| NKp30 | + | — | SEQ ID | SEQ ID | + | — | SEQ ID | SEQ ID | + | + |
| L30V/A60V/S64P/ | | | NO: 215 | NO: 235 | | | NO: 201 | NO: 233 | | |
| S86G | | | | | | | | | | |
| ICOSL | | | | | | | | | | |
| N52H/N57Y/Q100P | | | | | | | | | | |
| ICOSL WT | + | — | SEQ ID | SEQ ID | + | — | SEQ ID | SEQ ID | + | + |
| Nkp30 WT | | | NO: 196 | NO: 233 | | | NO: 214 | NO: 235 | | |
| ICOSL N52D | + | — | SEQ ID | SEQ ID | + | — | SEQ ID | SEQ ID | + | + |
| NKp30 | | | NO: 199 | NO: 233 | | | NO: 215 | NO: 235 | | |
| L30V/A60V/S64P/ | | | | | | | | | | |
| S86G | | | | | | | | | | |
| ICOSL | + | — | SEQ ID | SEQ ID | + | — | SEQ ID | SEQ ID | + | + |
| N52H/N57Y/Q100P | | | NO: 201 | NO: 233 | | | NO: 215 | NO: 235 | | |
| NKp30 | | | | | | | | | | |
| L30V/A60V/S64P/ | | | | | | | | | | |
| S86G | | | | | | | | | | |
| Domain 1: NKp30 | + | — | SEQ ID | SEQ ID | + | — | SEQ ID | SEQ ID | + | + |
| WT | | | NO: 214 | NO: 235 | | | NO: 152 | NO: 231 | | |
| Domain 2: CD80 | | | | | | | | | | |
| WT | | | | | | | | | | |
| Domain 1: NKp30 | + | — | SEQ ID | SEQ ID | + | SEQ | SEQ ID | SEQ ID | + | + |
| WT | | | NO: 214 | NO: 235 | | ID | NO: 220 | NO: 237 | | |
| Domain 2: CD86 | | | | | | NO: | | | | |
| WT | | | | | | 236 | | | | |
| Domain 1: NKp30 | + | — | SEQ ID | SEQ ID | + | — | SEQ ID | SEQ ID | + | + |
| L30V/A60V/S64P/ | | | NO: 215 | NO: 235 | | | NO: 192 | NO: 231 | | |
| S86G | | | | | | | | | | |
| Domain 2: CD80 | | | | | | | | | | |
| R29H/Y31H/T41G/ | | | | | | | | | | |
| Y87N/E88G/K89E/ | | | | | | | | | | |
| D90N/A91G/P109S | | | | | | | | | | |
| Domain 1: NKp30 | + | — | SEQ ID | SEQ ID | + | — | SEQ ID | SEQ ID | + | + |
| L30V/A60V/S64P/ | | | NO: 215 | NO: 235 | | | NO: 175 | NO: 231 | | |
| S86G | | | | | | | | | | |
| Domain 2: CD80 | | | | | | | | | | |
| I67T/L70Q/A91G/ | | | | | | | | | | |
| T120S | | | | | | | | | | |
| Domain 1: NKp30 | + | — | SEQ ID | SEQ ID | + | SEQ | SEQ ID | SEQ ID | + | + |
| L30V/A60V/S64P/ | | | NO: 215 | NO: 235 | | ID | NO: 221 | NO: 237 | | |
| S86G | | | | | | NO: | | | | |
| Domain 2: CD86 | | | | | | 236 | | | | |
| Q35H/H90L/Q102H | | | | | | | | | | |
| Domain 1: CD80 | + | — | SEQ ID | SEQ ID | + | — | SEQ ID | SEQ ID | + | + |
| WT | | | NO: 152 | NO: 231 | | | NO: 214 | NO: 235 | | |
| Domain 2: Nkp30 | | | | | | | | | | |
| WT | | | | | | | | | | |
| Domain 1: CD86 | + | SEQ | SEQ ID | SEQ ID | + | — | SEQ ID | SEQ ID | + | + |
| WT | | ID | NO: 220 | NO: 237 | | | NO: 214 | NO: 235 | | |
| Domain 2: Nkp30 | | NO: | | | | | | | | |
| WT | | 236 | | | | | | | | |
| Domain 1: CD80 | + | — | SEQ ID | SEQ ID | + | — | SEQ ID | SEQ ID | + | + |
| R29H/Y31H/T41G/ | | | NO: 192 | NO: 231 | | | NO: 215 | NO: 235 | | |
| Y87N/E88G/K89E/ | | | | | | | | | | |
| D90N/A91G/P109S | | | | | | | | | | |
| Domain 2: NKp30 | | | | | | | | | | |
| L30V/A60V/S64P/ | | | | | | | | | | |
| S86G | | | | | | | | | | |
| Domain 1: CD80 | + | — | SEQ ID | SEQ ID | + | — | SEQ ID | SEQ ID | + | + |
| I67T/L70Q/A91G/ | | | NO: 175 | NO: 231 | | | NO: 215 | NO: 235 | | |
| T120S | | | | | | | | | | |
| Domain 2: NKp30 | | | | | | | | | | |
| L30V/A60V/S64P/ | | | | | | | | | | |
| S86G | | | | | | | | | | |
| Domain 1: CD86 | + | SEQ | SEQ ID | SEQ ID | + | — | SEQ ID | SEQ ID | + | + |
| Q35H/H90L/Q102H | | ID | NO: 221 | NO: 237 | | | NO: 215 | NO: 235 | | |
| Domain 2: NKp30 | | NO: | | | | | | | | |
| L30V/A60V/S64P/ | | 236 | | | | | | | | |
| S86G | | | | | | | | | | |
| Domain 1: CD80 | + | — | SEQ ID | SEQ ID | + | — | SEQ ID | SEQ ID | + | + |
| WT | | | NO: 152 | NO: 231 | | | NO: 196 | NO: 233 | | |
| Domain 2: ICOSL | | | | | | | | | | |
| WT | | | | | | | | | | |
| Domain 1: CD80 | + | — | SEQ ID | SEQ ID | + | SEQ | SEQ ID | SEQ ID | + | + |
| WT | | | NO: 152 | NO: 231 | | ID | NO: 220 | NO: 237 | | |
| Domain 2: CD86 | | | | | | NO: | | | | |
| WT | | | | | | 236 | | | | |
| Domain 1: CD80 | + | — | SEQ ID | SEQ ID | + | — | SEQ ID | SEQ ID | + | + |
| WT | | | NO: 152 | NO: 231 | | | NO: 152 | NO: 231 | | |
| Domain 2: CD80 | | | | | | | | | | |
| WT | | | | | | | | | | |
| Domain 1: CD80 | + | — | SEQ ID | SEQ ID | + | — | SEQ ID | SEQ ID | + | + |
| E88D/K89R/D90K/ | | | NO: 189 | NO: 231 | | | NO: 192 | NO: 231 | | |
| A91G/F92Y/K93R | | | | | | | | | | |
| Domain 2: CD80 | | | | | | | | | | |
| R29H/Y31H/T41G/ | | | | | | | | | | |
| Y87N/E88G/K89E/ | | | | | | | | | | |
| D90N/A91G/P109S | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 192 | NO: 231 | | |
| F59L/E77K/P109S/ | | | | | | | | | | |
| I118T | | | | | | | | | | |
| Domain 2: CD80 | | | | | | | | | | |
| R29H/Y31H/T41G/ | | | | | | | | | | |
| Y87N/E88G/K89E/ | | | | | | | | | | |
| D90N/A91G/P109S | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 175 | NO: 231 | | |
| F59L/E77K/P109S/ | | | | | | | | | | |
| I118T | | | | | | | | | | |
| Domain 2: CD80 | | | | | | | | | | |
| I67T/L70Q/A91G/ | | | | | | | | | | |
| T120S | | | | | | | | | | |
| Domain 1: CD80 | + | — | SEQ ID | SEQ ID | + | SEQ | SEQ ID | SEQ ID | + | + |
| E88D/K89R/D90K/ | | | NO: 189 | NO: 231 | | ID | NO: 221 | NO: 237 | | |
| A91G/F92Y/K93R | | | | | | NO: | | | | |
| Domain 2: CD86 | | | | | | 236 | | | | |
| Q35H/H90L/Q102H | | | | | | | | | | |
| Domain 1: CD80 | + | — | SEQ ID | SEQ ID | + | SEQ | SEQ ID | SEQ ID | + | + |
| A12T/H18L/N43V/ | | | NO: 193 | NO: 231 | | ID | NO: 221 | NO: 237 | | |
| F59L/E77K/P109S/ | | | | | | NO: | | | | |
| I118T | | | | | | 236 | | | | |
| Domain 1: CD86 | | | | | | | | | | |
| Q35H/H90L/Q102H | | | | | | | | | | |
| Domain 1: CD80 | + | — | SEQ ID | SEQ ID | + | — | SEQ ID | SEQ ID | + | + |
| E88D/K89R/D90K/ | | | NO: 189 | NO: 231 | | | NO: 213 | NO: 233 | | |
| A91G/F92Y/K93R | | | | | | | | | | |
| Domain 2: ICOSL | | | | | | | | | | |
| N52S/N57Y/H94D/ | | | | | | | | | | |
| L96F/L98F/Q100R/ | | | | | | | | | | |
| G103E/ | | | | | | | | | | |
| F120S | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 213 | NO: 233 | | |
| F59L/E77K/P109S/ | | | | | | | | | | |
| I118T | | | | | | | | | | |
| Domain 2: ICOSL | | | | | | | | | | |
| N52S/N57Y/H94D/ | | | | | | | | | | |
| L96F/L98F/Q100R/ | | | | | | | | | | |
| G103E/ | | | | | | | | | | |
| F120S | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 199 | NO: 233 | | |
| F59L/E77K/P109S/ | | | | | | | | | | |
| I118T | | | | | | | | | | |
| Domain 2: ICOSL | | | | | | | | | | |
| N52D | | | | | | | | | | |
| Domain 1: CD80 | + | — | SEQ ID | SEQ ID | + | — | SEQ ID | SEQ ID | + | + |
| E88D/K89R/D90K/ | | | NO: 189 | NO: 231 | | | NO: 201 | NO: 233 | | |
| A91G/F92Y/K93R | | | | | | | | | | |
| Domain 2: ICOSL | | | | | | | | | | |
| N52H/N57Y/Q100P | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 201 | NO: 233 | | |
| F59L/E77K/P109S/ | | | | | | | | | | |
| I118T | | | | | | | | | | |
| Domain 2: ICOSL | | | | | | | | | | |
| N52H/N57Y/Q100P | | | | | | | | | | |
| Domain 1: ICOSL | + | — | SEQ ID | SEQ ID | + | — | SEQ ID | SEQ ID | + | + |
| WT | | | NO: 196 | NO: 233 | | | NO: 152 | NO: 231 | | |
| Domain 2: CD80 | | | | | | | | | | |
| WT | | | | | | | | | | |
| Domain 1: CD86 | + | SEQ | SEQ ID | SEQ ID | + | — | SEQ ID | SEQ ID | + | + |
| WT | | ID | NO: 220 | NO: 237 | | | NO: 152 | NO: 231 | | |
| Domain 2: CD80 | | NO: | | | | | | | | |
| WT | | 236 | | | | | | | | |
| Domain 1: CD80 | + | — | SEQ ID | SEQ ID | + | — | SEQ ID | SEQ ID | + | + |
| R29H/Y31H/T41G/ | | | NO: 192 | NO: 231 | | | NO: 189 | NO: 231 | | |
| Y87N/E88G/K89E/ | | | | | | | | | | |
| D90N/A91G/P109S | | | | | | | | | | |
| Domain 2: CD80 | | | | | | | | | | |
| E88D/K89R/D90K/ | | | | | | | | | | |
| A91G/F92Y/K93R | | | | | | | | | | |
| Domain 1: CD80 | + | — | SEQ ID | SEQ ID | + | — | SEQ ID | SEQ ID | + | + |
| R29H/Y31H/T41G/ | | | NO: 192 | NO: 231 | | | NO: 193 | NO: 231 | | |
| Y87N/E88G/K89E/ | | | | | | | | | | |
| D90N/A91G/P109S | | | | | | | | | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: CD80 | + | — | SEQ ID | SEQ ID | + | — | SEQ ID | SEQ ID | + | + |
| I67T/L70Q/A91G/ | | | NO: 175 | NO: 231 | | | NO: 189 | NO: 231 | | |
| T120S | | | | | | | | | | |
| Domain 2: CD80 | | | | | | | | | | |
| E88D/K89R/D90K/ | | | | | | | | | | |
| A91G/F92Y/K93R | | | | | | | | | | |
| Domain 1: CD80 | + | — | SEQ ID | SEQ ID | + | — | SEQ ID | SEQ ID | + | + |
| I67T/L70Q/A91G/ | | | NO: 175 | NO: 231 | | | NO: 193 | NO: 231 | | |
| T120S | | | | | | | | | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: CD86 | + | SEQ | SEQ ID | SEQ ID | + | — | SEQ ID | SEQ ID | + | + |
| Q35H/H90L/Q102H | | ID | NO: 221 | NO: 237 | | | NO: 189 | NO: 231 | | |
| Domain 2: CD80 | | NO: | | | | | | | | |
| E88D/K89R/D90K/ | | 236 | | | | | | | | |
| A91G/F92Y/K93R | | | | | | | | | | |
| Domain 1: CD86 | + | SEQ | SEQ ID | SEQ ID | + | — | SEQ ID | SEQ ID | + | + |
| Q35H/H90L/Q102H | | ID | NO: 221 | NO: 237 | | | NO: 193 | NO: 231 | | |
| Domain 2: CD80 | | NO: | | | | | | | | |
| A12T/H18L/N43V/ | | 236 | | | | | | | | |
| F59L/E77K/P109S/ | | | | | | | | | | |
| I118T | | | | | | | | | | |
| Domain 1: ICOSL | + | — | SEQ ID | SEQ ID | + | — | SEQ ID | SEQ ID | + | + |
| N52S/N57Y/H94D/ | | | NO: 213 | NO: 233 | | | NO: 189 | NO: 231 | | |
| L96F/L98F/Q100R/ | | | | | | | | | | |
| G103E/ | | | | | | | | | | |
| F120S | | | | | | | | | | |
| Domain 2: CD80 | | | | | | | | | | |
| E88D/K89R/D90K/ | | | | | | | | | | |
| A91G/F92Y/K93R | | | | | | | | | | |
| Domain 1: ICOSL | + | — | SEQ ID | SEQ ID | + | — | SEQ ID | SEQ ID | + | + |
| N52S/N57Y/H94D/ | | | NO: 213 | NO: 233 | | | NO: 193 | NO: 231 | | |
| L96F/L98F/Q100R/ | | | | | | | | | | |
| G103E/ | | | | | | | | | | |
| F120S | | | | | | | | | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: ICOSL | + | — | SEQ ID | SEQ ID | + | — | SEQ ID | SEQ ID | + | + |
| N52D | | | NO: 199 | NO: 233 | | | NO: 189 | NO: 231 | | |
| Domain 2: CD80 | | | | | | | | | | |
| E88D/K89R/D90K/ | | | | | | | | | | |
| A91G/F92Y/K93R | | | | | | | | | | |
| Domain 1: ICOSL | + | — | SEQ ID | SEQ ID | + | — | SEQ ID | SEQ ID | + | + |
| N52D | | | NO: 199 | NO: 233 | | | NO: 193 | NO: 231 | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: ICOSL | + | — | SEQ ID | SEQ ID | + | — | SEQ ID | SEQ ID | + | + |
| N52H/N57Y/Q100P | | | NO: 201 | NO: 233 | | | NO: 189 | NO: 231 | | |
| Domain 2: CD80 | | | | | | | | | | |
| E88D/K89R/D90K/ | | | | | | | | | | |
| A91G/F92Y/K93R | | | | | | | | | | |
| Domain 1: ICOSL | + | — | SEQ ID | SEQ ID | + | — | SEQ ID | SEQ ID | + | + |
| N52H/N57Y/Q100P | | | NO: 201 | NO: 233 | | | NO: 193 | NO: 231 | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: CD80 | + | — | SEQ ID | SEQ ID | + | — | SEQ ID | SEQ ID | + | + |
| V68M/L70P/L72P/ | | | NO: 195 | NO: 231 | | | NO: 189 | NO: 231 | | |
| K86E | | | | | | | | | | |
| Domain 2: CD80 | | | | | | | | | | |
| E88D/K89R/D90K/ | | | | | | | | | | |
| A91G/F92Y/K93R | | | | | | | | | | |
| Domain 1: CD80 | + | — | SEQ ID | SEQ ID | + | — | SEQ ID | SEQ ID | + | + |
| R29V/Y31F/K36G/ | | | NO: 194 | NO: 231 | | | NO: 189 | NO: 231 | | |
| M38L/N43Q/E81R/ | | | | | | | | | | |
| V83I/L85I/K89R/ | | | | | | | | | | |
| D90L/A91E/F92N/ | | | | | | | | | | |
| K93Q/R94G | | | | | | | | | | |
| Domain 2: CD80 | | | | | | | | | | |
| E88D/K89R/D90K/ | | | | | | | | | | |
| A91G/F92Y/K93R | | | | | | | | | | |
| Domain 1: CD80 | + | — | SEQ ID | SEQ ID | + | — | SEQ ID | SEQ ID | + | + |
| V68M/L70P/L72P/ | | | NO: 195 | NO: 231 | | | NO: 193 | NO: 231 | | |
| K86E | | | | | | | | | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: CD80 | + | — | SEQ ID | SEQ ID | + | — | SEQ ID | SEQ ID | + | + |
| R29V/Y31F/K36G/ | | | NO: 194 | NO: 231 | | | NO: 193 | NO: 231 | | |
| M38L/N43Q/E81R/ | | | | | | | | | | |
| V83I/L85I/K89R/ | | | | | | | | | | |
| D90L/A91E/F92N/ | | | | | | | | | | |
| K93Q/R94G | | | | | | | | | | |
| Domain 2: CD80 | | | | | | | | | | |
| A12T/H18L/N43V/ | | | | | | | | | | |
| F59L/E77K/P109S/ | | | | | | | | | | |
| I118T | | | | | | | | | | |
| Domain 1: CD80 | + | — | SEQ ID | SEQ ID | + | — | SEQ ID | SEQ ID | + | + |
| E88D/K89R/D90K/ | | | NO: 189 | NO: 231 | | | NO: 195 | NO: 231 | | |
| A91G/F92Y/K93R | | | | | | | | | | |
| Domain 2: CD80 | | | | | | | | | | |
| V68M/L70P/L72P/ | | | | | | | | | | |
| K86E | | | | | | | | | | |
| Domain 1: CD80 | + | — | SEQ ID | SEQ ID | + | — | SEQ ID | SEQ ID | + | + |
| E88D/K89R/D90K/ | | | NO: 189 | NO: 231 | | | NO: 194 | NO: 231 | | |
| A91G/F92Y/K93R | | | | | | | | | | |
| Domain 2: CD80 | | | | | | | | | | |
| R29V/Y31F/K36G/ | | | | | | | | | | |
| M38L/N43Q/E81R/ | | | | | | | | | | |
| V83I/L85I/K89R/ | | | | | | | | | | |
| D90L/A91E/F92N/ | | | | | | | | | | |
| K93Q/R94G | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 195 | NO: 231 | | |
| F59L/E77K/P109S/ | | | | | | | | | | |
| I118T | | | | | | | | | | |
| Domain 2: CD80 | | | | | | | | | | |
| V68M/L70P/L72P/ | | | | | | | | | | |
| K86E | | | | | | | | | | |
| Domain 1: CD80 | + | — | SEQ ID | SEQ ID | + | — | SEQ ID | SEQ ID | + | + |
| A12T/H18L/N43V/ | | | NO: 193 | NO: 231 | | | NO: 194 | NO: 231 | | |
| F59L/E77K/P109S/ | | | | | | | | | | |
| I118T | | | | | | | | | | |
| Domain 2: CD80 | | | | | | | | | | |
| R29V/Y31F/K36G/ | | | | | | | | | | |
| M38L/N43Q/E81R/ | | | | | | | | | | |
| V83I/ | | | | | | | | | | |
| L85I/K89R/D90L/ | | | | | | | | | | |
| A91E/F92N/K93Q/ | | | | | | | | | | |
| R94G | | | | | | | | | | |
| Domain 1: CD86 | + | SEQ | SEQ ID | SEQ ID | + | — | SEQ ID | SEQ ID | + | + |
| WT | | ID | NO: 220 | NO: 237 | | | NO: 196 | NO: 233 | | |
| Domain 2: ICOSL | | NO: | | | | | | | | |
| WT | | 236 | | | | | | | | |
| Domain 1: CD80 | + | — | SEQ ID | SEQ ID | + | — | SEQ ID | SEQ ID | + | + |
| R29H/Y31H/T41G/ | | | NO: 192 | NO: 231 | | | NO: 213 | NO: 233 | | |
| Y87N/E88G/K89E/ | | | | | | | | | | |
| D90N/A91G/P109S | | | | | | | | | | |
| Domain 2: ICOSL | | | | | | | | | | |
| N52S/N57Y/H94D/ | | | | | | | | | | |
| L96F/L98F/Q100R/ | | | | | | | | | | |
| G103E/ | | | | | | | | | | |
| F120S | | | | | | | | | | |
| Domain 1: CD80 | + | — | SEQ ID | SEQ ID | + | — | SEQ ID | SEQ ID | + | + |
| I67T/L70Q/A91G/ | | | NO: 175 | NO: 231 | | | NO: 213 | NO: 233 | | |
| T120S | | | | | | | | | | |
| Domain 2: ICOSL | | | | | | | | | | |
| N52S/N57Y/H94D/ | | | | | | | | | | |
| L96F/L98F/Q100R/ | | | | | | | | | | |
| G103E/ | | | | | | | | | | |
| F120S | | | | | | | | | | |
| Domain 1: CD80 | + | — | SEQ ID | SEQ ID | + | — | SEQ ID | SEQ ID | + | + |
| R29H/Y31H/T41G/ | | | NO: 192 | NO: 231 | | | NO: 199 | NO: 233 | | |
| Y87N/E88G/K89E/ | | | | | | | | | | |
| D90N/A91G/P109S | | | | | | | | | | |
| Domain 2: ICOSL | | | | | | | | | | |
| N52D | | | | | | | | | | |
| Domain 1: CD80 | + | — | SEQ ID | SEQ ID | + | — | SEQ ID | SEQ ID | + | + |
| I67T/L70Q/A91G/ | | | NO: 175 | NO: 231 | | | NO: 199 | NO: 233 | | |
| T120S | | | | | | | | | | |
| Domain 2: ICOSL | | | | | | | | | | |
| N52D | | | | | | | | | | |
| Domain 1: CD80 | + | — | SEQ ID | SEQ ID | + | — | SEQ ID | SEQ ID | + | + |
| R29H/Y31H/T41G/ | | | NO: 192 | NO: 231 | | | NO: 201 | NO: 233 | | |
| Y87N/E88G/K89E/ | | | | | | | | | | |
| D90N/A91G/P109S | | | | | | | | | | |
| Domain 2: ICOSL | | | | | | | | | | |
| N52H/N57Y/Q100P | | | | | | | | | | |
| Domain 1: CD80 | + | — | SEQ ID | SEQ ID | + | — | SEQ ID | SEQ ID | + | + |
| I67T/L70Q/A91G/ | | | NO: 175 | NO: 231 | | | NO: 201 | NO: 233 | | |
| T190S | | | | | | | | | | |
| Domain 2: ICOSL | | | | | | | | | | |
| N52H/N57Y/Q100P | | | | | | | | | | |
| Domain 1: CD86 | + | SEQ | SEQ ID | SEQ ID | + | — | SEQ ID | SEQ ID | + | + |
| Q35H/H90L/Q102H | | ID | NO: 221 | NO: 237 | | | NO: 213 | NO: 233 | | |
| Domain 2: ICOSL | | NO: | | | | | | | | |
| N52S/N57Y/H94D/ | | 236 | | | | | | | | |
| L96F/L98F/Q100R/ | | | | | | | | | | |
| G103E/ | | | | | | | | | | |
| F120S | | | | | | | | | | |
| Domain 1: CD86 | + | SEQ | SEQ ID | SEQ ID | + | — | SEQ ID | SEQ ID | + | + |
| Q35H/H90L/Q102H | | ID | NO: 221 | NO: 237 | | | NO: 199 | NO: 233 | | |
| Domain 2: ICOSL | | NO: | | | | | | | | |
| N52D | | 236 | | | | | | | | |
| Domain 1: CD86 | + | SEQ | SEQ ID | SEQ ID | + | — | SEQ ID | SEQ ID | + | + |
| Q35H/H90L/Q102H | | ID | NO: 221 | NO: 237 | | | NO: 201 | NO: 233 | | |
| Domain 2: ICOSL | | NO: | | | | | | | | |
| N52H/N57Y/Q100P | | 236 | | | | | | | | |
| Domain 1: ICOSL | + | — | SEQ ID | SEQ ID | + | SEQ | SEQ ID | SEQ ID | + | + |
| WT | | | NO: 196 | NO: 233 | | ID | NO: 220 | NO: 237 | | |
| Domain 2: CD86 | | | | | | NO: | | | | |
| WT | | | | | | 236 | | | | |
| Domain 1: ICOSL | + | — | SEQ ID | SEQ ID | + | — | SEQ ID | SEQ ID | + | + |
| N52S/N57Y/H94D/ | | | NO: 213 | NO: 233 | | | NO: 192 | NO: 231 | | |
| L96F/L98F/Q100R/ | | | | | | | | | | |
| G103E/ | | | | | | | | | | |
| F120S | | | | | | | | | | |
| Domain 2: CD80 | | | | | | | | | | |
| R29H/Y31H/T41G/ | | | | | | | | | | |
| Y87N/E88G/K89E/ | | | | | | | | | | |
| D90N/A91G/P109S | | | | | | | | | | |
| Domain 1: ICOSL | + | — | SEQ ID | SEQ ID | + | — | SEQ ID | SEQ ID | + | + |
| N52S/N57Y/H94D/ | | | NO: 213 | NO: 233 | | | NO: 175 | NO: 231 | + | |
| L96F/L98F/Q100R/ | | | | | | | | | | |
| G103E/ | | | | | | | | | | |
| F120S | | | | | | | | | | |
| Domain 2: CD80 | | | | | | | | | | |
| I67T/L70Q/A91G/ | | | | | | | | | | |
| T120S | | | | | | | | | | |
| Domain 1: ICOSL | + | — | SEQ ID | SEQ ID | + | — | SEQ ID | SEQ ID | + | + |
| N52D | | | NO: 199 | NO: 233 | | | NO: 192 | NO: 231 | | |
| Domain 2: CD80 | | | | | | | | | | |
| R29H/Y31H/T41G/ | | | | | | | | | | |
| Y87N/E88G/K89E/ | | | | | | | | | | |
| D90N/A91G/P109S | | | | | | | | | | |
| Domain 1: ICOSL | + | — | SEQ ID | SEQ ID | + | — | SEQ ID | SEQ ID | + | + |
| N52D | | | NO: 199 | NO: 233 | | | NO: 175 | NO: 231 | | |
| Domain 2: CD80 | | | | | | | | | | |
| I67T/L70Q/A91G/ | | | | | | | | | | |
| T120S | | | | | | | | | | |
| Domain 1: ICOSL | + | — | SEQ ID | SEQ ID | + | — | SEQ ID | SEQ ID | + | + |
| N52H/N57Y/Q100P | | | NO: 201 | NO: 233 | | | NO: 192 | NO: 231 | | |
| Domain 2: CD80 | | | | | | | | | | |
| R29H/Y31H/T41G/ | | | | | | | | | | |
| Y87N/E88G/K89E/ | | | | | | | | | | |
| D90N/A91G/P109S | | | | | | | | | | |
| Domain 1: ICOSL | + | — | SEQ ID | SEQ ID | + | SEQ | SEQ ID | SEQ ID | + | + |
| N52S/N57Y/H94D/ | | | NO: 213 | NO: 233 | | ID | NO: 221 | NO: 237 | | |
| L96F/L98F/Q100R/ | | | | | | NO: | | | | |
| G103E/ | | | | | | 236 | | | | |
| F120S | | | | | | | | | | |
| Domain 2: CD86 | | | | | | | | | | |
| Q35H/H90L/Q102H | | | | | | | | | | |
| Domain 1: ICOSL | + | — | SEQ ID | SEQ ID | + | SEQ | SEQ ID | SEQ ID | + | + |
| N52D | | | NO: 199 | NO: 233 | | ID | NO: 221 | NO: 237 | | |
| Domain 2: CD86 | | | | | | NO: | | | | |
| Q35H/H90L/Q102H | | | | | | 236 | | | | |
| Domain 1: ICOSL | + | — | SEQ ID | SEQ ID | + | SEQ | SEQ ID | SEQ ID | + | + |
| N52H/N57Y/Q100P | | | NO: 201 | NO: 233 | | ID | NO: 221 | NO: 237 | | |
| Domain 2: CD86 | | | | | | NO: | | | | |
| Q35H/H90L/Q102H | | | | | | 236 | | | | |

High throughput expression and purification of the variant IgV-stacked-Fc fusion molecules containing various combinations of variant IgV domains from CD80, CD86, ICOSL or Nkp30 containing at least one affinity-modified IgV domain were generated as described in Example 5. Binding of the variant IgV-stacked-Fc fusion molecules to respective counter structures and functional activity by anti-CD3 coimmobilization assay also were assessed as described in Example 6. For example, costimulatory bioactivity of the stacked IgSF Fc fusion proteins was determined in a similar immobilized anti-CD3 assay as above. In this case, 4 nM of anti-CD3 (OKT3, Biolegend, USA) was coimmobilized with 4 nM to 120 nM of human rB7-H6.Fc (R&D Systems, USA) or human rPD-L1.Fc (R&D Systems, USA) overnight on tissue-culture treated 96 well plates (Corning, USA). The following day unbound protein was washed off with PBS and 100,000 purified pan T cells were added to each well in 100 ul Ex-Vivo 15 media (Lonza, Switzerland). The stacked IgSF domains were subsequently added at concentrations ranging from 8 nM to 40 nM in a volume of 100 ul for 200 ul volume total. Cells were cultured 3 days before harvesting culture supernatants and measuring human IFN-gamma levels with Duoset ELISA kit (R&D Systems, USA) as mentioned above.

The results are set forth in Tables 12-16. Specifically, Table 12 sets forth binding and functional activity results for variant IgV-stacked-Fc fusion molecules containing an NKp30 IgV domain and an ICOSL IgV domain. Table 13 sets forth binding and functional activity results for variant IgV-stacked-Fv fusion molecules containing an Nkp30 IgV domain and a CD80 or CD86 IgV domain. Table 14 sets forth binding and functional activity results for variant IgV-stacked-Fc fusion molecules containing a variant CD80 IgV domain and a CD80, CD86 or ICOSL IgV domain. Table 15 sets forth binding and functional activity results for variant IgV-stacked-Fc fusion molecules containing two variant CD80 IgV domains. Table 16 sets forth results for variant IgV-stacked Fc fusion molecules containing a variant CD80 or CD86 IgV domain and a variant ICOSL IgV domain.

For each of Tables 12-16, Column 1 indicates the structural organization and orientation of the stacked, affinity modified or wild-type (WT) domains beginning with the amino terminal (N terminal) domain, followed by the middle WT or affinity modified domain located before the C terminal human IgG1 Fc domains. Column 2 sets forth the SEQ ID NO identifier for the sequence of each IgV domain contained in a respective “stack” molecule. Column 3 shows the binding partners which the indicated affinity modified stacked domains from column 1 were selected against.

Also shown is the binding activity as measured by the Mean Fluorescence Intensity (MFI) value for binding of each stack molecule to cells engineered to express various counter structure ligands and the ratio of the MFI compared to the binding of the corresponding stack molecule containing unmodified IgV domains not containing the amino acid substitution(s) to the same cell-expressed counter structure ligand. The functional activity of the variant stack molecules to modulate the activity of T cells also is shown based on the calculated levels of IFN-gamma in culture supernatants (pg/ml) generated with the indicated variant stack molecule in solution and the appropriate ligand coimmoblized with anti-CD3 as described in Example 6. The Tables also depict the ratio of IFN-gamma produced by each variant stack molecule compared to the corresponding unmodified stack molecule in the coimmobilization assay.

As shown, the results showed that it was possible to generate stack molecules containing at least one variant IgSF domains that exhibited affinity-modified activity of increased binding for at least one cognate counter structure ligand compared to a corresponding stack molecule containing the respective unmodified (e.g. wild-type) IgV domain. In some cases, the stack molecule, either from one or a combination of both variant IgSF domains in the molecule, exhibited increased binding for more than one cognate counter structure ligand. The results also showed that the order of the IgV domains in the stacked molecules could, in some cases, alter the degree of improved binding activity. In some cases, functional T cell activity also was altered when assessed in the targeted coimmobilization assay.

TABLE 12
Stacked variant IgV Fc fusion proteins containing an NKp30 IgV domain and an ICOSL IgV domain

| | | | | | | Anti-CD3 |
|---|---|---|---|---|---|---|
| | | | | | | coimmobilization |
| | | | Binding Activity | | | assay |
| | | Counter | B7H6 MFI | ICOS MFI | CD28 MFI | pg/ml IFN- |
| Domain Structure | SEQ ID | structure | (WT | (WT | (WT | gamma |
| N terminal to C terminal: | NO | selected | parental | parental | parental | (WT parental IFN- |
| domain 1/domain 2/Fc | (IgV) | against | MFI ratio) | MFI ratio) | MFI ratio) | gamma ratio) |
| Domain 1: NKp30 WT | 214 | — | 64538 | 26235 | 6337 | 235 |
| Domain 2: ICOSL WT | 196 | | (1.00) | (1.00) | (1.00) | (1.00) |
| Domain 1: NKp30 (L30V | 215 | B7-H6 | 59684 | 12762 | 9775 | 214 |
| A60V S64P S86G) | | | (0.92) | (0.49) | (1.54) | (0.91) |
| Domain 2: ICOSL (N52S | 212 | ICOS- | | | | |
| N57Y H94D L96F L98F | | CD28 | | | | |
| Q100R) | | | | | | |
| Domain 1: NKp30 (L30V | 215 | B7-H6 | 65470 | 30272 | 9505 | 219 |
| A60V S64P S86G) | | | (1.01) | (1.15) | (1.50) | (0.93) |
| Domain 2: ICOSL (N52D) | 199 | ICOS- | | | | |
| | | CD28 | | | | |
| Domain 1: NKp30 (L30V | 215 | B7-H6 | 38153 | 27903 | 11300 | 189 |
| A60V S64P S86G)/ | | | (0.59) | (1.06) | (1.78) | (0.80) |
| Domain 2: ICOSL (N52H | 201 | ICOS- | | | | |
| N57Y Q100P) | | CD28 | | | | |
| Domain 1: ICOSL WT | 196 | — | 117853 | 70320 | 7916 | 231 |
| Domain 2: Nkp30 WT | 214 | | (1.0) | (1.0) | (1.0) | (1.0) |
| Domain 1: ICOSL (N52D) | 199 | ICOS- | 100396 | 83912 | 20778 | 228 |
| | | CD28 | (0.85) | (1.19) | (2.62) | (0.98) |
| Domain 2: NKp30 (L30V | 215 | B7-H6 | | | | |
| A60V S64P S86G) | | | | | | |
| Domain 1: ICOSL (N52H | 201 | ICOS- | 82792 | 68874 | 72269 | 561 |
| N57Y Q100P) | | CD28 | (0.70) | (0.98) | (9.12) | (2.43) |
| Domain 2: NKp30 (L30V | 215 | B7-H6 | | | | |
| A60V S64P S86G) | | | | | | |

TABLE 13
Stacked variant IgV Fc fusion proteins containing an NKp30 IgV domain and a CD80 or CD86 IgV domain

| | | | | | | | Anti-CD3 | |
|---|---|---|---|---|---|---|---|---|
| | | | | | | | coimmobilization | |
| | | Counter | Binding Activity | | | | assay | |
| Domain Structure | SEQ ID | structure | B7H6 MFI | | CD28 MFI | | pg/ml IFN-gamma | |
| N terminal to C terminal: | NO | selected | (WT parental | | (WT parental | | (WT parental IFN- | |
| domain 1/domain 2/Fc | (IgV) | against | MFI ratio) | | MFI ratio) | | gamma ratio) | |
| Domain 1: NKp30 WT | 214 | — | 88823 | (1.00) | 7022 | (1.00) | 68 | (1.00) |
| Domain 2: CD80 WT | 152 | | | | | | | |
| Domain 1: NKp30 WT | 214 | — | 14052 | (1.00) | 1690 | (1.00) | 92 | (1.00) |
| Domain 2: CD86 WT | 220 | | | | | | | |
| Domain 1: NKp30 (L30V | 215 | B7-H6 | 53279 | (0.60) | 9027 | (1.29) | 94 | (1.38) |
| A60V S64P S86G) | | | | | | | | |
| Domain 2: CD80 | 192 | CD28 | | | | | | |
| R29H/Y31H/T41G/Y87N/E88G/ | | | | | | | | |
| K89E/D90N/A91G/P109S | | | | | | | | |
| Domain 1: NKp30 (L30V | 215 | B7-H6 | 41370 | (0.47) | 11240 | (1.60) | 60 | (0.88) |
| A60V S64P S86G) | | | | | | | | |
| Domain 2: CD80 | 175 | CD28 | | | | | | |
| I67T/L70Q/A91G/T120S | | | | | | | | |
| Domain 1: NKp30 (L30V | 215 | B7-H6 | 68480 | (4.87) | 9115 | (5.39) | 110 | (1.19) |
| A60V S64P S86G)/ | | | | | | | | |
| Domain 2: CD86 | 221 | CD28 | | | | | | |
| Q35H/H90L/Q102H | | | | | | | | |
| Domain 1: CD80 WT | 152 | — | 110461 | (1.00) | 13654 | (1.00) | 288 | (1.00) |
| Domain 2: Nkp30 WT | 214 | | | | | | | |
| Domain 1: CD86 WT | 220 | CD28 | 128899 | (1.00) | 26467 | (1.00) | 213 | (1.00) |
| Domain 2: Nkp30 WT | 214 | B7-H6 | | | | | | |
| Domain 1: CD80 | 192 | CD28 | 55727 | (0.50) | 4342 | (0.32) | 100 | (0.35) |
| R29H/Y31H/T41G/Y87N/E88G/ | | | | | | | | |
| K89E/D90N/A91G/P109S | | | | | | | | |
| Domain 2: NKp30 (L30V | 215 | B7-H6 | | | | | | |
| A60V S64P S86G) | | | | | | | | |
| Domain 1: CD80 | 175 | CD28 | 40412 | (0.37) | 7094 | (0.52) | 84 | (0.29) |
| I67T/L70Q/A91G/T120S | | | | | | | | |
| Domain 2: NKp30 (L30V | 215 | B7-H6 | | | | | | |
| A60V S64P S86G) | | | | | | | | |
| Domain 1: CD86 | 221 | CD28 | 220836 | (1.71) | 11590 | (0.44) | 113 | (0.53) |
| Q35H/H90L/Q102H | | | | | | | | |
| Domain 2: NKp30 (L30V | 215 | B7-H6 | | | | | | |
| A60V S64P S86G) | | | | | | | | |

TABLE 14
Stacked variant IgV Fc fusion proteins containing a CD80 IgV domain and a CD80, CD86, or ICOSL IgV domain

| | | | | | | Anti-CD3 |
|---|---|---|---|---|---|---|
| | | | Binding Activity | | | coimmobilization |
| | | | | PD-L1 | | assay |
| | | Counter | CD28 MFI | MFI | ICOS MFI | pg/ml IFN- |
| Domain Structure | SEQ ID | structure | (WT | (WT | (WT | gamma |
| N terminal to C terminal: | NO | selected | parental | parental | parental | (WT parental IFN- |
| domain 1/domain 2/Fc | (IgV) | against | MFI ratio) | MFI ratio) | MFI ratio) | gamma ratio) |
| Domain 1: CD80 WT | 152 | | 1230 | 2657 | 11122 | 69 |
| Domain 2: ICOSL WT | 196 | | (1.00) | (1.00) | (1.00) | (1.00) |
| Domain 1: CD80 WT | 152 | | 60278 | 2085 | | 59 |
| Domain 2: CD86 WT | 220 | | (1.00) | (1.00) | | (1.00) |
| Domain 1: CD80 WT | 152 | | 1634 | 6297 | | 98 |
| Domain 2: CD80 WT | 152 | | (1.00) | (1.00) | | (1.00) |
| Domain 1: CD80 | 189 | PD-L1 | 4308 | 4234 | | 214 |
| E88D/K89R/D90K/A91G/F92Y/ | | | (2.64) | (0.67) | | (2.18) |
| K93R | | | | | | |
| Domain 2: CD80 | 192 | CD28 | | | | |
| R29H/Y31H/T41G/Y87N/E88G/ | | | | | | |
| K89E/D90N/A91G/P109S | | | | | | |
| Domain 1: CD80 | 193 | PD-L1 | 7613 | 2030 | | 137 |
| A12T/H18L/N43V/F59L/E77K/ | | | (4.66) | (0.32) | | (1.40) |
| P109S/I118T | | | | | | |
| Domain 2: CD80 | 192 | CD28 | | | | |
| R29H/Y31H/T41G/Y87N/E88G/ | | | | | | |
| K89E/D90N/A91G/P109S | | | | | | |
| Domain 1: CD80 | 193 | PD-L1 | 3851 | 3657 | | 81 |
| A12T/H18L/N43V/F59L/E77K/ | | | (2.36) | (0.58) | | (0.83) |
| P109S/I118T | | | | | | |
| Domain 2: CD80 | 175 | CD28 | | | | |
| I67T/L70Q/A91G/T120S | | | | | | |
| Domain 1: CD80 | 189 | PD-L1 | 4117 | 2914 | | 96 |
| E88D/K89R/D90K/A91G/F92Y/ | | | (0.07) | (1.40) | | (1.63) |
| K93R | | | | | | |
| Domain 2: CD86 | 221 | CD28 | | | | |
| Q35H/H90L/Q102H | | | | | | |
| Domain 1: CD80 | 193 | PD-L1 | 2868 | 3611 | | 94 |
| A12T/H18L/N43V/F59L/E77K/ | | | (0.05) | (1.73) | | (1.60) |
| P109S/I118T | | | | | | |
| Domain 2: CD86 | 221 | CD28 | | | | |
| Q35H/H90L/Q102H | | | | | | |
| Domain 1: CD80 | 189 | PD-L1 | 3383 | 4515 | 5158 | 90 |
| E88D/K89R/D90K/A91G/F92Y/ | | | (2.75) | (1.70) | (0.46) | (1.30) |
| K93R | | | | | | |
| Domain 2: ICOSL | 213 | ICOS/CD28 | | | | |
| N52S/N57Y/H94D/L96F/L98F/ | | | | | | |
| Q100R/G103E/ | | | | | | |
| F120S | | | | | | |
| Domain 1: CD80 | 193 | PD-L1 | 2230 | 2148 | 3860 | 112 |
| A12T/H18L/N43V/F59L/E77K/ | | | (1.81) | (0.81) | (0.35) | (1.62) |
| P109S/I118T | | | | | | |
| Domain 2: ICOSL | 213 | ICOS/CD28 | | | | |
| N52S/N57Y/H94D/L96F/L98F/ | | | | | | |
| Q100R/G103E/ | | | | | | |
| F120S | | | | | | |
| Domain 1: CD80 | 193 | PD-L1 | 5665 | 6446 | 15730 | 126 |
| A12T/H18L/N43V/F59L/E77K/ | | | (4.61) | (2.43) | (1.41) | (1.83) |
| P109S/I118T | | | | | | |
| Domain 2: ICOSL | 199 | ICOS/CD28 | | | | |
| N52D | | | | | | |
| Domain 1: CD80 | 189 | PD-L1 | 6260 | 4543 | 11995 | 269 |
| E88D/K89R/D90K/A91G/F92Y/ | | | (5.09) | (1.71) | (1.08) | (3.90) |
| K93R | | | | | | |
| Domain 2: ICOSL | 201 | ICOS/CD28 | | | | |
| N52H/N57Y/Q100P | | | | | | |
| Domain 1: CD80 | 193 | PD-L1 | 3359 | 3874 | 8541 | 97 |
| A12T/H18L/N43V/F59L/E77K/ | | | (2.73) | (1.46) | (0.77) | (1.41) |
| P109S/I118T | | | | | | |
| Domain 2: ICOSL | 201 | ICOS/CD28 | | | | |
| N52H/N57Y/Q100P | | | | | | |
| Domain 1: ICOSL WT | 196 | | 3000 | 2966 | 14366 | 101 |
| Domain 2: CD80 WT | 152 | | (1.00) | (1.00) | (1.00) | (1.00) |
| Domain 1: CD86 WT | 220 | | 4946 | 1517 | | 125 |
| Domain 2: CD80 WT | 152 | | (1.00) | (1.00) | | (1.00) |
| Domain 1: CD80 | 192 | CD28 | 2832 | 3672 | | 114 |
| R29H/Y31H/T41G/Y87N/E88G/ | | | (1.73) | (0.58) | | (1.16) |
| K89E/D90N/A91G/P109S | | | | | | |
| Domain 2: CD80 | 189 | PD-L1 | | | | |
| E88D/K89R/D90K/A91G/F92Y/ | | | | | | |
| K93R | | | | | | |
| Domain 1: CD80 | 192 | CD28 | 4542 | 2878 | | 142 |
| R29H/Y31H/T41G/Y87N/E88G/ | | | (2.78) | (0.45) | | (1.45) |
| K89E/D90N/A91G/P109S | | | | | | |
| Domain 2: CD80 | 193 | PD-L1 | | | | |
| A12T/H18L/N43V/F59L/E77K/ | | | | | | |
| P109S/I118T | | | | | | |
| Domain 1: CD80 | 175 | CD28 | 938 | 995 | | 102 |
| I67T/L70Q/A91G/T120S | | | (0.57) | (0.16) | | (1.04) |
| Domain 2: CD80 | 189 | PD-L1 | | | | |
| E88D/K89R/D90K/A91G/F92Y/ | | | | | | |
| K93R | | | | | | |
| Domain 1: CD80 | 175 | CD28 | 4153 | 2827 | | 108 |
| I67T/L70Q/A91G/T120S | | | (2.54) | (0.45) | | (1.10) |
| Domain 2: CD80 | 193 | PD-L1 | | | | |
| A12T/H18L/N43V/F59L/E77K/ | | | | | | |
| P109S/I118T | | | | | | |
| Domain 1: CD86 | 221 | CD28 | 14608 | 2535 | | 257 |
| Q35H/H90L/Q102H | | | (2.95) | (1.67) | | (2.06) |
| Domain 2: CD80 | 189 | PD-L1 | | | | |
| E88D/K89R/D90K/A91G/F92Y/ | | | | | | |
| K93R | | | | | | |
| Domain 1: CD86 | 221 | CD28 | 2088 | 2110 | | 101 |
| Q35H/H90L/Q102H | | | (0.42) | (1.39) | | (0.81) |
| Domain 2: CD80 | 193 | PD-L1 | | | | |
| A12T/H18L/N43V/F59L/E77K/ | | | | | | |
| P109S/I118T | | | | | | |
| Domain 1: ICOSL | 213 | ICOS/CD28 | 3634 | 4893 | 6403 | 123 |
| N52S/N57Y/H94D/L96F/L98F/ | | | (1.21) | (1.65) | (0.45) | (1.22) |
| Q100R/G103E/ | | | | | | |
| F120S | | | | | | |
| Domain 2: CD80 | 189 | PD-L1 | | | | |
| E88D/K89R/D90K/A91G/F92Y/ | | | | | | |
| K93R | | | | | | |
| Domain 1: ICOSL | 213 | ICOS/CD28 | 1095 | 5929 | 7923 | 127 |
| N52S/N57Y/H94D/L96F/L98F/ | | | (0.37) | (2.0) | (0.55) | (1.26) |
| Q100R/G103E/ | | | | | | |
| F120S | | | | | | |
| Domain 2: CD80 | 193 | PD-L1 | | | | |
| A12T/H18L/N43V/F59L/E77K/ | | | | | | |
| P109S/I118T | | | | | | |
| Domain 1: ICOSL | 199 | ICOSL/CD28 | 2023 | 5093 | 16987 | 125 |
| N52D | | | (0.67) | (1.72) | (1.18) | (1.24) |
| Domain 2: CD80 | 189 | PD-L1 | | | | |
| E88D/K89R/D90K/A91G/F92Y/ | | | | | | |
| K93R | | | | | | |
| Domain 1: ICOSL | 199 | ICOS/CD28 | 3441 | 3414 | 20889 | 165 |
| N52D | | | (1.15) | (1.15) | (1.45) | (1.63) |
| Domain 2: CD80 | 193 | PD-L1 | | | | |
| A12T/H18L/N43V/F59L/E77K/ | | | | | | |
| P109S/I118T | | | | | | |
| Domain 1: ICOSL | 201 | ICOS/CD28 | 7835 | 6634 | 20779 | 95 |
| N52H/N57Y/Q100P | | | (2.61) | (2.24) | (1.45) | (0.94) |
| Domain 2: CD80 | 189 | PD-L1 | | | | |
| E88D/K89R/D90K/A91G/F92Y/ | | | | | | |
| K93R | | | | | | |
| Domain 1: ICOSL | 201 | ICOS/CD28 | 8472 | 3789 | 13974 | 106 |
| N52H/N57Y/Q100P | | | (2.82) | (1.28) | (0.97) | (1.05) |
| Domain 2: CD80 | 193 | PD-L1 | | | | |
| A12T/H18L/N43V/F59L/E77K/ | | | | | | |
| P109S/I118T | | | | | | |

TABLE 15
Stacked variant IgV Fc fusion proteins containing two CD80 IgV domains

| | | Counter | Binding Activity | | | | Functional | |
|---|---|---|---|---|---|---|---|---|
| Domain Structure | SEQ ID | structure | PD-L1 MFI | | CTLA-4 MFI | | Activity MLR | |
| N terminal to C terminal: | NO | selected | (WT parental | | (WT parental | | IFN-gamma | |
| domain 1/domain 2/Fc | (IgV) | against | MFI ratio) | | MFI ratio) | | pg/ml | |
| Domain 1: CD80 WT | 152 | | 6297 | (1.00) | 4698 | (1.00) | 35166 | (1.00) |
| Domain 2: CD80 WT | 152 | | | | | | | |
| Domain 1: CD80 | 195 | CTLA-4 | 2464 | (0.39) | 4955 | (1.05) | 5705 | (0.16) |
| V68M/L70P/L72P/K86E | | | | | | | | |
| Domain 2: CD80 | 189 | PD-L1 | | | | | | |
| E88D/K89R/D90K/A91G/ | | | | | | | | |
| F92Y/K93R | | | | | | | | |
| Domain 1: CD80 | 194 | CTLA-4 | 1928 | (0.31) | 1992 | (0.42) | 1560 | (0.04) |
| R29V/Y31F/K36G/M38L/N43Q/ | | | | | | | | |
| E81R/V83I/L85I/K89R/D90L/ | | | | | | | | |
| A91E/F92N/K93Q/R94G | | | | | | | | |
| Domain 2: CD80 | 189 | PD-L1 | | | | | | |
| E88D/K89R/D90K/A91G/ | | | | | | | | |
| F92Y/K93R | | | | | | | | |
| Domain 1: CD80 | 195 | CTLA-4 | 1215 | (0.19) | 1382 | (0.29) | 2171 | (0.06) |
| V68M/L70P/L72P/K86E | | | | | | | | |
| Domain 2: CD80 | 193 | PD-L1 | | | | | | |
| A12T/H18L/N43V/F59L/ | | | | | | | | |
| E77K/P109S/I118T | | | | | | | | |
| Domain 1: CD80 | 194 | CTLA-4 | 1592 | (0.25) | 1962 | (0.42) | 1512 | (0.04) |
| R29V/Y31F/K36G/M38L/N43Q/ | | | | | | | | |
| E81R/V83I/L85I/K89R/D90L/ | | | | | | | | |
| A91E/F92N/K93Q/R94G | | | | | | | | |
| Domain 2: CD80 | 193 | PD-L1 | | | | | | |
| A12T/H18L/N43V/F59L/E77K/ | | | | | | | | |
| P109S/I118T | | | | | | | | |
| Domain 1: CD80 | 189 | PD-L1 | 1747 | (0.28) | 2057 | (0.44) | 9739 | (0.28) |
| E88D/K89R/D90K/A91G/ | | | | | | | | |
| F92Y/K93R | | | | | | | | |
| Domain 2: CD80 | 195 | CTLA-4 | | | | | | |
| V68M/L70P/L72P/K86E | | | | | | | | |
| Domain 1: CD80 | 189 | PD-L1 | 1752 | (0.28) | 1772 | (0.38) | 5412 | (0.15) |
| E88D/K89R/D90K/A91G/ | | | | | | | | |
| F92Y/K93R | | | | | | | | |
| Domain 2: CD80 | 194 | CTLA-4 | | | | | | |
| R29V/Y31F/K36G/M38L/N43Q/ | | | | | | | | |
| E81R/V83I/L85I/K89R/D90L/ | | | | | | | | |
| A91E/F92N/K93Q/R94G | | | | | | | | |
| Domain 1: CD80 | 193 | PD-L1 | 1636 | (0.26) | 1887 | (0.40) | 7608 | (0.22) |
| A12T/H18L/N43V/F59L/E77K/ | | | | | | | | |
| P109S/I118T | | | | | | | | |
| Domain 2: CD80 | 195 | CTLA-4 | | | | | | |
| V68M/L70P/L72P/K86E | | | | | | | | |
| Domain 1: CD80 | 193 | PD-L1 | 2037 | (0.32) | 4822 | (1.03) | 11158 | (0.32) |
| A12T/H18L/N43V/F59L/E77K/ | | | | | | | | |
| P109S/I118T | | | | | | | | |
| Domain 2: CD80 | 194 | CTLA-4 | | | | | | |
| R29V/Y31F/K36G/M38L/N43Q/ | | | | | | | | |
| E81R/V83I/L85I/K89R/D90L/ | | | | | | | | |
| A91E/F92N/K93Q/R94G | | | | | | | | |

TABLE 16
Stacked variant IgV Fc fusion proteins containing a CD80 or CD86 IgV domain and an ICOSL IgV domain

| | | Counter | Binding Activity | | | | Functional | |
|---|---|---|---|---|---|---|---|---|
| Domain Structure | SEQ ID | structure | PD-L1 MFI | | CTLA-4 MFI | | Activity MLR | |
| N terminal to C terminal: | NO | selected | (WT parental | | (WT parental | | IFN-gamma | |
| domain 1/domain 2/Fc | (IgV) | against | MFI ratio) | | MFI ratio) | | pg/ml | |
| Domain 1: CD80 WT | 152 | | 1230 | (1.00) | 11122 | (1.00) | 1756 | (1.00) |
| Domain 2: ICOSL WT | 196 | | | | | | | |
| Domain 1: CD86 WT | 220 | | 29343 | (1.00) | 55193 | (1.00) | 6305 | (1.00) |
| Domain 2: ICOSL WT | 196 | | | | | | | |
| Domain 1: CD80 | 192 | CD28 | 2280 | (1.85) | 3181 | (0.29) | 2281 | (1.30) |
| R29H/Y31H/T41G/Y87N/E88G/ | | | | | | | | |
| K89E/D90N/A91G/P109S | | | | | | | | |
| Domain 2: ICOSL | 213 | ICOS/CD28 | | | | | | |
| N52S/N57Y/H94D/L96F/L98F/ | | | | | | | | |
| Q100R/G103E/F120S | | | | | | | | |
| Domain 1: CD80 | 175 | CD28 | 2309 | (1.88) | 26982 | (2.43) | 1561 | (0.89) |
| I67T/L70Q/A91G/T120S | | | | | | | | |
| Domain 2: ICOSL | 213 | ICOS/CD28 | | | | | | |
| N52S/N57Y/H94D/L96F/L98F/ | | | | | | | | |
| Q100R/G103E/F120S | | | | | | | | |
| Domain 1: CD80 | 192 | CD28 | 4285 | (3.48) | 22744 | (2.04) | 1612 | (0.92) |
| R29H/Y31H/T41G/Y87N/E88G/ | | | | | | | | |
| K89E/D90N/A91G/P109S | | | | | | | | |
| Domain 2: ICOSL | 199 | ICOS/CD28 | | | | | | |
| N52D | | | | | | | | |
| Domain 1: CD80 | 175 | CD28 | 3024 | (2.46) | 16916 | (1.52) | 3857 | (2.20) |
| I67T/L70Q/A91G/T120S | | | | | | | | |
| Domain 2: ICOSL | 199 | ICOS/CD28 | | | | | | |
| N52D | | | | | | | | |
| Domain 1: CD80 | 192 | CD28 | 6503 | (5.29) | 7240 | (0.65) | 6886 | (3.92) |
| R29H/Y31H/T41G/Y87N/E88G/ | | | | | | | | |
| K89E/D90N/A91G/P109S | | | | | | | | |
| Domain 2: ICOSL | 201 | ICOS/CD28 | | | | | | |
| N52H/N57Y/Q100P | | | | | | | | |
| Domain 1: CD80 | 175 | CD28 | 3110 | (2.53) | 4848 | (0.44) | 3393 | (1.93) |
| I67T/L70Q/A91G/T120S | | | | | | | | |
| Domain 2: ICOSL | 201 | ICOS/CD28 | | | | | | |
| N52H/N57Y/Q100P | | | | | | | | |
| Domain 1: CD86 | 221 | CD28 | | | | | | |
| Q35H/H90L/Q102H | | | | | | | | |
| Domain 2: ICOSL | 213 | ICOS/CD28 | 11662 | (0.40) | 21165 | (0.38) | 880 | (0.14) |
| N52S/N57Y/H94D/L96F/L98F/ | | | | | | | | |
| Q100R/G103E/F120S | | | | | | | | |
| Domain 1: CD86 | 221 | CD28 | 24230 | (0.83) | 73287 | (1.33) | 1110 | (0.18) |
| Q35H/H90L/Q102H | | | | | | | | |
| Domain 2: ICOSL | 199 | ICOS/CD28 | | | | | | |
| N52D | | | | | | | | |
| Domain 1: CD86 | 221 | CD28 | 1962 | (0.07) | 1630 | (0.03) | 587 | (0.09) |
| Q35H/H90L/Q102H | | ICOS/CD28 | | | | | | |
| Domain 2: ICOSL | 201 | | | | | | | |
| N52H/N57Y/Q100P | | | | | | | | |
| Domain 1: ICOSL WT | 196 | | 3000 | (1.00) | 14366 | (1.00) | 4113 | (1.00) |
| Domain 2: CD80 WT | 152 | | | | | | | |
| Domain 1: ICOSL WT | 196 | | 18005 | (1.00) | 53602 | (1.00) | 18393 | (1.00) |
| Domain 2: CD86 WT | 220 | | | | | | | |
| Domain 1: ICOSL | 213 | ICOSL/CD28 | 10426 | (3.48) | 51286 | (3.57) | 18680 | (4.54) |
| N52S/N57Y/H94D/L96F/L98F/ | | | | | | | | |
| Q100R/G103E/F120S | | | | | | | | |
| Domain 2: CD80 | 192 | CD28 | | | | | | |
| R29H/Y31H/T41G/Y87N/E88G/ | | | | | | | | |
| K89E/D90N/A91G/P109S | | | | | | | | |
| Domain 1: ICOSL | 213 | ICOS/CD28 | 17751 | (5.92) | 29790 | (2.07) | 10637 | (2.59) |
| N52S/N57Y/H94D/L96F/L98F/ | | | | | | | | |
| Q100R/G103E/F120S | | | | | | | | |
| Domain 2: CD80 | 175 | CD28 | | | | | | |
| I67T/L70Q/A91G/T120S | | | | | | | | |
| Domain 1: ICOSL | 199 | ICOS/CD28 | 2788 | (0.93) | 25870 | (1.80) | 6205 | (1.51) |
| N52D | | | | | | | | |
| Domain 2: CD80 | 192 | CD28 | | | | | | |
| R29H/Y31H/T41G/Y87N/E88G/ | | | | | | | | |
| K89E/D90N/A91G/P109S | | | | | | | | |
| Domain 1: ICOSL | 199 | ICOS/CD28 | 2522 | (0.84) | 13569 | (0.94) | 5447 | (1.32) |
| N52D | | | | | | | | |
| Domain 2: CD80 | 175 | CD28 | | | | | | |
| I67T/L70Q/A91G/T120S | | | | | | | | |
| Domain 1: ICOSL | 201 | ICOS/CD28 | | | | | | |
| N52H/N57Y/Q100P | | | | | | | | |
| Domain 2: CD80 | 192 | CD28 | 9701 | (3.23) | 9187 | (0.64) | 5690 | (1.38) |
| R29H/Y31H/T41G/Y87N/E88G/ | | | | | | | | |
| K89E/D90N/A91G/P109S | | | | | | | | |
| Domain 1: ICOSL | 213 | ICOS/CD28 | 27050 | (1.50) | 21257 | (0.40) | 8131 | (0.44) |
| N52S/N57Y/H94D/L96F/L98F/ | | | | | | | | |
| Q100R/G103E/F120S | | | | | | | | |
| Domain 2: CD86 | 221 | CD28 | | | | | | |
| Q35H/H90L/Q102H | | | | | | | | |
| Domain 1: ICOSL | 199 | ICOS/CD28 | 34803 | (1.93) | 80210 | (1.50) | 6747 | (0.37) |
| N52D | | | | | | | | |
| Domain 2: CD86 | 221 | CD28 | | | | | | |
| Q35H/H90L/Q102H | | | | | | | | |
| Domain 1: ICOSL | 201 | ICOS/CD28 | 5948 | (0.33) | 4268 | (0.08) | 26219 | (1.43) |
| N52H/N57Y/Q100P | | | | | | | | |
| Domain 2: CD86 | 221 | CD28 | | | | | | |
| Q35H/H90L/Q102H | | | | | | | | |

### Example 9: Generation, Selection and Screening of Affinity-Modified IgSF Domain Variants of CD155

Affinity-modified IgSF domain variants of CD155 were generated substantially as described in Examples 1-6 above with some slight modifications. For example, for the generation of CD155 variants, only the IgV domain, and not the other two domains of the ECD, was included in the generated proteins.

To generate a library targeting specific residues of CD155 by complete or partial randomization with degenerate codons, the coding DNA for the immunoglobulin-like V-type (IgV) domain of human CD155 (SEQ ID NO:241) was ordered from Integrated DNA Technologies (Coralville, Iowa) as a set of overlapping oligonucleotides of up to 80 base pairs (bp) in length. In general, to generate a library of diverse variants of the IgV domain, the oligonucleotides contained desired degenerate codons at desired amino acid positions. Degenerate codons were generated using an algorithm at the URL: rosettadesign.med.unc.edu/SwiftLib/. In general, positions to mutate and degenerate codons were chosen from crystal structure information (PDB: 3UDW) or homology models built from this structure containing the target-ligand pairs of interest to identify ligand contact residues as well as residues that are at the protein interaction interface. For example, a crystal structure of CD155 bound to TIGIT is publicly available at the URL: www.rcsb.org/pdb/explore/explore.do?structureId=3UDW for Protein Data Base code 3UDW. This analysis was performed using a structure viewer available at the URL: spdbv.vital-it.ch).

The oligonucleotides were used for PCR amplification to generate library DNA inserts for insertion into the modified yeast display version of vector pBYDS03 substantially as described in Example 1. Alternatively, Ultramers (Integrated DNA Technologies) of up to 200 bp in length were used in conjunction with megaprimer PCR (URL: http://www.ncbi.nlm.nih.gov/pmc/articles/PMC146891/pdf/253371.pdf) to generate larger stretches of degenerate codons that could not be as easily incorporated using multiple small overlapping primers. Following the generation of full length product using megaprimer PCR, the mutant IgV domain library was PCR amplified again using DNA primers containing 40 bp overlap region with the modified pBYDS03 cloning variant for homologous recombination into yeast. The library of DNA inserts were prepared for library insertion substantially as descried in Example 1 and electroporation-ready DNA was prepared.

As alternative approaches, either sublibraries generated by site-directed mutagenesis to target specific residues of the IgV domain of CD155 or random libraries of the IgV domain of CD155 were generated to further identify variants of the IgV domain of CD155 substantially as described in Example 1.

The degenerate or random library DNA was inserted into yeast substantially as described in Example 2. Yeast expressing affinity modified variants of CD155 were selected using the method substantially described in Example 3. For the selection, the following target ligand proteins were employed: human rTIGIT.Fc (i.e., recombinant TIGIT-Fc fusion protein) and rCD226.Fc. Magnetic Protein A beads were obtained from New England Biolabs, USA. For two-color, flow cytometric sorting, a Bio-Rad S3e sorter was used. CD155 display levels were monitored with an anti-hemagglutinin antibody labeled with Alexafluor 488 (Life Technologies, USA). Ligand binding of Fc fusion proteins, rTIGIT.Fc or rCD226.Fc, were detected with PE conjugated human Ig specific goat Fab (Jackson ImmunoResearch, USA). Doublet yeast were gated out using forward scatter (FSC)/side scatter (SSC) parameters, and sort gates were based upon higher ligand binding detected in FL2 that possessed more limited tag expression binding in FL1.

Second sort outputs (F2) were obtained substantially as described in Example 3 by expanding and re-inducing expression of sort outputs that had been assayed for higher specific binding affinity and were used to assess binding compared to the parental, wild-type yeast strain. For CD155, the second FACS outputs (F2) were compared to parental CD155 yeast for binding rTIGIT.Fc or rCD226.Fc by double staining each population with anti-HA (hemagglutinin) tag expression and the anti-human Fc secondary to detect ligand binding.

Selected outputs were reformatted as immunomodulatory proteins containing an affinity modified (variant) immunoglobulin-like V-type (IgV) domain of CD155 fused to an Fc molecule (variant IgV domain-Fc fusion molecules) substantially as described in Example 4, except including only the IgV domain and not the full ECD domain. In some alternative methods for the CD155 outputs, DNA from the outputs were PCR amplified with primers containing 40 bp overlap regions on either end with an Fc fusion vector to carry out in vitro recombination using Gibson Assembly Mastermix (New England Biolabs), which was subsequently used in heat shock transformation into E. Coli strain NEB5alpha. Exemplary of an Fc fusion vector is pFUSE-hIgG1-Fc2 (Invivogen, USA).

After transformation, samples were prepared for DNA sequencing substantially as described in Example 4. The sequences were then manually curated as described in Example 4, except that they were manually curated so that they start at the beginning of the IgV coding region. The curated sequences were batch-translated and aligned as described in Example 4. Clones of interest were identified using the criteria as described in Example 4. Table 17 sets forth the identified variant CD155 affinity-modified molecules, including the amino acid substitutions contained in each variant.

The methods generated immunomodulatory proteins containing an affinity-modified IgV domain of CD155 in which the encoding DNA was generated to encode a protein as follows: signal peptide followed by variant (mutant) IgV domain followed by a linker of three alanines (AAA) followed by a human IgG1 Fc containing the mutation N297G (N82G with reference to wild-type human IgG1 Fc set forth in SEQ ID NO: 226). The human IgG1 Fc also contained the mutations R292C and V302C (corresponding to R77C and V87C with reference to wild-type human IgG1 Fc set forth in SEQ ID NO: 226). Since the construct does not include any antibody light chains that can form a covalent bond with a cysteine, the human IgG1 Fc also contained replacement of the cysteine residues to a serine residue at position 5 (C5S) compared to the wild-type or unmodified Fc set forth in SEQ ID NO: 226.

Recombinant variant CD155 Fc fusion proteins were expressed and purified substantially as described in Example 5. Binding and activity of the affinity-modified variant CD155 Fc fusion proteins was assessed substantially as described in Example 6, except that cells expressing full length human CD226 and TIGIT cognate binding partners were generated. Cells were stained by flow cytometry with CD155 Fc variant and mean fluorescence intensity (MFI) was determined as described in Example 5. For bioactivity characterization, recombinant CD155 Fc variants were assessed in an anti-CD3 coimmobilization assay substantially as described in Example 5.

The results for the binding and activity studies are set forth in Table 17. The Table indicates exemplary IgSF domain amino acid substitutions (replacements) in the IgV domain of CD155 selected in the screen for affinity-maturation against the respective cognate structures TIGIT and CD226. The exemplary amino acid substitutions are designated by amino acid position number corresponding to position of the unmodified sequence set forth in SEQ ID NO: 241 (IgV). The amino acid position is indicated in the middle, with the corresponding unmodified (e.g. wild-type) amino acid listed before the number and the identified variant amino acid substitution listed after the number. Column 2 sets forth the SEQ ID NO identifier for the variant for each variant ECD-Fc fusion molecule.

Also shown is the binding activity as measured by the Mean Fluorescence Intensity (MFI) value for binding of each variant Fc-fusion molecule to cells engineered to express the cognate counter structure ligand as the ratio of the MFI compared to the binding of the corresponding unmodified ECD-Fc fusion molecule not containing the amino acid substitution(s) to the same cell-expressed counter structure ligand. The functional activity of the variant Fc-fusion molecules to modulate the activity of T cells also is shown based on the calculated levels of IFN-gamma in culture supernatants (pg/ml) generated with the indicated variant Fc fusion molecule coimmoblized with anti-CD3 as a ratio of IFN-gamma produced by each variant CD155 IgV-Fc compared to the corresponding unmodified CD155 IgV-Fc in both functional assays.

TABLE 17
CD155 variants selected against cognate binding partners. Molecule sequences, binding data,
and costimulatory bioactivity data.

| | | | | | | Anti-CD3 |
|---|---|---|---|---|---|---|
| | | CD226 | | CD96 | Mock | IFN-gamma |
| | | tfxn MFI | TIGIT tfxn | MFI | Expi293 | (pg/ml) |
| | | (CD226 | MFI | (CD96 | MFI | (Anti-CD3 |
| | SEQ | MFI | (TIGIT MFI | MFI | (Mock MFI | IFN-gamma |
| | ID NO | parental | parental | parental | parental | parental |
| CD155 mutations | (IgV) | ratio) | ratio) | ratio) | ratio) | ratio) |
| P18S, P64S, F91S | 264 | 497825 | 247219 | 140065 | 3528 | 270.1 |
| | | (133.7) | (91.1) | (45.4) | (1.2) | (0.7) |
| P18S, F91S, L104P | 265 | 26210 | 75176 | 10867 | 2130 | 364.2 |
| | | (7.0) | (27.7) | (3.5) | (0.7) | (0.9) |
| L44P | 266 | 581289 | 261931 | 152252 | 3414 | 277.6 |
| | | (156.1) | (96.5) | (49.4) | (1.2) | (0.7) |
| A56V | 267 | 455297 | 280265 | 161162 | 2601 | 548.2 |
| | | (122.3) | (103.2) | (52.2) | (0.9) | (1.4) |
| P18L, L79V, F91S | 268 | 5135 | 4073 | 3279 | 2719 | 1241.5 |
| | | (1.4) | (1.5) | (1.1) | (0.9) | (3.2) |
| P18S, F91S | 269 | 408623 | 284190 | 147463 | 3348 | 760.6 |
| | | (109.8) | (104.7) | (47.8) | (1.1) | (2.0) |
| P18T, F91S | 270 | 401283 | 223985 | 157644 | 3065 | 814.7 |
| | | (107.8) | (82.5) | (51.1) | (1.1) | (2.1) |
| P18T, S42P, F91S | 271 | 554105 | 223887 | 135395 | 3796 | 539.7 |
| | | (148.8) | (82.5) | (43.9) | (1.3) | (1.4) |
| G7E, P18T, Y30C, F91S | 272 | 12903 | 12984 | 7906 | 2671 | 275.9 |
| | | (3.5) | (4.8) | (2.6) | (0.9) | (0.7) |
| P18T, F91S, G111D | 273 | 438327 | 287315 | 167583 | 4012 | 307.2 |
| | | (117.7) | (105.8) | (54.3) | (1.4) | (0.8) |
| P18S, F91P | 274 | 4154 | 3220 | 2678 | 2816 | 365.7 |
| | | (1.1) | (1.2) | (0.9) | (1.0) | (0.9) |
| P18T, F91S, F108L | 275 | 394546 | 298680 | 193122 | 2926 | 775.4 |
| | | (106.0) | (110.0) | (62.6) | (1.0) | (2.0) |
| P18T, T45A, F91S | 277 | 435847 | 222044 | 191026 | 2948 | 1546.8 |
| | | (117.1) | (81.8) | (61.9) | (1.0) | (4.0) |
| P18T, F91S, R94H | 278 | 3589 | 2942 | 2509 | 2390 | 1273.2 |
| | | (1.0) | (1.1) | (0.8) | (0.8) | (3.3) |
| P18S, Y30C, F91S | 279 | 382352 | 276358 | 56934 | 3540 | 426.5 |
| | | (102.7) | (101.8) | (18.5) | (1.2) | (1.1) |
| A81V, L83P | 280 | 4169 | 2912 | 2616 | 2993 | 339.7 |
| | | (1.1) | (1.1) | (0.8) | (1.0) | (0.9) |
| L88P | 281 | 65120 | 74845 | 35280 | 2140 | 969.2 |
| | | (17.5) | (27.6) | (11.4) | (0.7) | (2.5) |
| Wild type | 241 | 3723 | 2715 | 3085 | 2913 | 389.6 |
| | | (1.0) | (1.0) | (1.0) | (1.0) | (1.0) |
| R94H | 282 | 18905 | 104013 | 11727 | 1663 | 372.6 |
| | | (5.1) | (38.3) | (3.8) | (0.6) | (1.0) |
| A13E, P18S, A56V, | 283 | 357808 | 179060 | 118570 | 2844 | 349.2 |
| F91S | | (96.1) | (66.0) | (38.4) | (1.0) | (0.9) |
| P18T, F91S, V115A | 284 | 38487 | 46313 | 22718 | 2070 | 1574.5 |
| | | (10.3) | (17.1) | (7.4) | (0.7) | (4.0) |
| P18T, Q60K | 285 | 238266 | 173730 | 154448 | 4778 | 427.2 |
| | | (64.0) | (64.0) | (50.1) | (1.6) | (1.1) |

### Example 10: Generation, Selection and Screening of Affinity-Modified IgSF Domain Variants of CD112

Affinity-modified IgSF domain variants of CD112 were generated substantially as described in Examples 1-6 above with some slight modifications. For example, for the generation of CD112 variants, only the IgV domain, and not the other two domains of the ECD, was included in the generated proteins.

To generate a library targeting specific residues of CD155 by complete or partial randomization with degenerate codons, the coding DNA for the immunoglobulin-like V-type (IgV) domain of human CD112 (SEQ ID NO:286) was ordered from Integrated DNA Technologies (Coralville, Iowa) as a set of overlapping oligonucleotides of up to 80 base pairs (bp) in length. In general, to generate a library of diverse variants of the IgV domain, the oligonucleotides contained desired degenerate codons at desired amino acid positions. Degenerate codons were generated using an algorithm at the URL: rosettadesign.med.unc.edu/SwiftLib/. In general, positions to mutate and degenerate codons were chosen from crystal structure information (PDB: 3UDW) or homology models built from this structure containing the target-ligand pairs of interest to identify ligand contact residues as well as residues that are at the protein interaction interface.

The oligonucleotides were used for PCR amplification to generate library DNA inserts for insertion into the modified yeast display version of vector pBYDS03 substantially as described in Example 1. Alternatively, Ultramers (Integrated DNA Technologies) of up to 200 bp in length were used in conjunction with megaprimer PCR (URL: http://www.ncbi.nlm.nih.gov/pmc/articles/PMC146891/pdf/253371.pdf) to generate larger stretches of degenerate codons that could not be as easily incorporated using multiple small overlapping primers. Following the generation of full length product using megaprimer PCR, the mutant IgV domain library was PCR amplified again using DNA primers containing 40 bp overlap region with the modified pBYDS03 cloning variant for homologous recombination into yeast. The library of DNA inserts were prepared for library insertion substantially as descried in Example 1 and electroporation-ready DNA was prepared.

As alternative approaches, either sublibraries generated by site-directed mutagenesis to target specific residues of the IgV domain of CD112 or random libraries of the IgV domain of CD112 were generated to further identify variants of the IgV domain of CD112 substantially as described in Example 1.

The degenerate or random library DNA was inserted into yeast substantially as described in Example 2. Yeast expressing affinity modified variants of CD112 were selected using the method substantially described in Example 3. For the selection, the following target ligand proteins were employed: human rTIGIT.Fc (i.e., recombinant TIGIT-Fc fusion protein), rCD226.Fc and rCD112R.Fc. Magnetic Protein A beads were obtained from New England Biolabs, USA. For two-color, flow cytometric sorting, a Bio-Rad S3e sorter was used. CD112 display levels were monitored with an anti-hemagglutinin antibody labeled with Alexafluor 488 (Life Technologies, USA). Ligand binding of Fc fusion proteins, rTIGIT.Fc, rCD226.Fc, or rCD112R.Fc, were detected with PE conjugated human Ig specific goat Fab (Jackson ImmunoResearch, USA). Doublet yeast were gated out using forward scatter (FSC)/side scatter (SSC) parameters, and sort gates were based upon higher ligand binding detected in FL2 that possessed more limited tag expression binding in FL1.

Second sort outputs (F2) were obtained substantially as described in Example 3 by expanding and re-inducing expression of sort outputs that had been assayed for higher specific binding affinity and were used to assess binding compared to the parental, wild-type yeast strain. For CD112, the second FACS outputs (F2) were compared to parental CD112 yeast for binding of each rTIGIT.Fc, rCD226.Fc, and rCD112R by double staining each population with anti-HA (hemagglutinin) tag expression and the anti-human Fc secondary to detect ligand binding.

Selected outputs were reformatted as immunomodulatory proteins containing an affinity modified (variant) immunoglobulin-like V-type (IgV) domain of CD112 fused to an Fc molecule (variant IgV domain-Fc fusion molecules) substantially as described in Example 4, except including only the IgV domain and not the full ECD domain. In some alternative methods for the CD112 outputs, DNA from the outputs were PCR amplified with primers containing 40 bp overlap regions on either end with an Fc fusion vector to carry out in vitro recombination using Gibson Assembly Mastermix (New England Biolabs), which was subsequently used in heat shock transformation into E. Coli strain NEB5alpha. Exemplary of an Fc fusion vector is pFUSE-hIgG1-Fc2 (Invivogen, USA).

After transformation, samples were prepared for DNA sequencing substantially as described in Example 4. The sequences were then manually curated as described in Example 4, except that they were manually curated so that they start at the beginning of the IgV coding region. The curated sequences were batch-translated and aligned as described in Example 4. Clones of interest were identified using the criteria as described in Example 4. Table 18 sets forth the identified variant CD112 affinity-modified molecules, including the amino acid substitutions contained in each variant.

The methods generated immunomodulatory proteins containing an affinity-modified IgV domain of CD112 in which the encoding DNA was generated to encode a protein as follows: signal peptide followed by variant (mutant) IgV domain followed by a linker of three alanines (AAA) followed by a human IgG1 Fc containing the mutation N297G (N82G with reference to wild-type human IgG1 Fc set forth in SEQ ID NO: 226). The human IgG1 Fc also contained the mutations R292C and V302C (corresponding to R77C and V87C with reference to wild-type human IgG1 Fc set forth in SEQ ID NO: 226). Since the construct does not include any antibody light chains that can form a covalent bond with a cysteine, the human IgG1 Fc also contained replacement of the cysteine residues to a serine residue at position 5 (C5S) compared to the wild-type or unmodified Fc set forth in SEQ ID NO: 226.

Recombinant variant CD112 Fc fusion proteins were expressed and purified substantially as described in Example 5. Binding and activity of the affinity-modified variant CD112 Fc fusion proteins was assessed substantially as described in Example 6, except that cells expressing full length human CD226, TIGIT and CD112R cognate binding partners were generated. Cells were stained by flow cytometry with CD112 Fc variant and mean fluorescence intensity (MFI) was determined as described in Example 5. For bioactivity characterization, recombinant CD112 Fc variants were assessed in an anti-CD3 coimmobilization assay substantially as described in Example 5.

The results for the binding and activity studies are set forth in Table 18. The Table indicates exemplary IgSF domain amino acid substitutions (replacements) in the IgV domain of CD112 selected in the screen for affinity-maturation against the respective cognate structures TIGIT, CD226 and CD112R. The exemplary amino acid substitutions are designated by amino acid position number corresponding to position of the unmodified sequence set forth in SEQ ID NO: 286. The amino acid position is indicated in the middle, with the corresponding unmodified (e.g. wild-type) amino acid listed before the number and the identified variant amino acid substitution listed after the number. Column 2 sets forth the SEQ ID NO identifier for the variant ECD for each variant ECD-Fc fusion molecule.

Also shown is the binding activity as measured by the Mean Fluorescence Intensity (MFI) value for binding of each variant Fc-fusion molecule to cells engineered to express the cognate counter structure ligand as the ratio of the MFI compared to the binding of the corresponding unmodified ECD-Fc fusion molecule not containing the amino acid substitution(s) to the same cell-expressed counter structure ligand. The functional activity of the variant Fc-fusion molecules to modulate the activity of T cells also is shown based on the calculated levels of IFN-gamma in culture supernatants (pg/ml) generated with the indicated variant Fc fusion molecule coimmoblized with anti-CD3 as a ratio of IFN-gamma produced by each variant CD112 IgV-Fc compared to the corresponding unmodified CD112 IgV-Fc in both functional assays.

TABLE 18
CD112 variants selected against cognate binding partners. Molecule sequences, binding data,
and costimulatory bioactivity data.

| | | | | | | Anti-CD3 |
|---|---|---|---|---|---|---|
| | | TIGIT | CD112R | CD226 | | IFN-gamma |
| | | tfxn MFI | tfxn MFI | MFI | Mock Expi293 | (pg/ml) |
| | | (TIGIT | (CD112R | (CD226 | MFI | (Anti-CD3 |
| | SEQ | MFI | MFI | MFI | (Mock MFI | IFN-gamma |
| | ID NO | parental | parental | parental | parental | parental |
| CD112 mutations | (IgV) | ratio) | ratio) | ratio) | ratio) | ratio) |
| WT CD112 | 286 | 210829 | 1452 | 265392 | 1112 | 676.6 |
| | | (1.00) | (1.00) | (1.00) | (1.00) | (1.00) |
| Y33H, A112V, G117D | 334 | 12948 | 1552 | 1368 | 1241 | 164.8 |
| | | (0.06) | (1.07) | (0.01) | (1.12) | (0.24) |
| V19A, Y33H, S64G, S80G, | 335 | 48356 | 1709 | 2831 | 1098 | |
| G98S, N106Y, A112V | | (0.23) | (1.18) | (0.01) | (0.99) | |
| L32P, A112V | 336 | 191432 | 1557 | 11095 | 1259 | 390.4 |
| | | (0.91) | (1.07) | (0.04) | (1.13) | (0.58) |
| A95V, A112I | 337 | 238418 | 1706 | 51944 | 1215 | 282.5 |
| | | (1.13) | (1.17) | (0.20) | (1.09) | (0.42) |
| P28S, A112V | 338 | 251116 | 1985 | 153382 | 1189 | 503.4 |
| | | (1.19) | (1.37) | (0.58) | (1.07) | (0.74) |
| P27A, T38N, V101A, | 339 | 255803 | 2138 | 222822 | 1399 | 240.7 |
| A112V | | (1.21) | (1.47) | (0.84) | (1.26) | (0.36) |
| S118F | 340 | 11356 | 5857 | 6938 | 1270 | 271.7 |
| | | (0.05) | (4.03) | (0.03) | (1.14) | (0.40) |
| R12W, H48Y, F54S, S118F | 341 | 10940 | 3474 | 5161 | 1069 | |
| | | (0.05) | (2.39) | (0.02) | (0.96) | |
| R12W, Q79R, S118F | 342 | 2339 | 7370 | 1880 | 1338 | 447.4 |
| | | (0.01) | (5.08) | (0.01) | (1.20) | (0.66) |
| T113S, S118Y | 343 | 6212 | 6823 | 1554 | 1214 | 225.1 |
| | | (0.03) | (4.70) | (0.01) | (1.09) | (0.33) |
| S118Y | 344 | 2921 | 6535 | 2003 | 1463 | 190.4 |
| | | (0.01) | (4.50) | (0.01) | (1.32) | (0.28) |
| N106I, S118Y | 345 | 2750 | 7729 | 1815 | 1222 | 265.8 |
| | | (0.01) | (5.32) | (0.01) | (1.10) | (0.39) |
| N106I, S118F | 346 | 1841 | 9944 | 1529 | 1308 | 437.9 |
| | | (0.01) | (6.85) | (0.01) | (1.18) | (0.65) |
| A95T, L96P, S118Y | 347 | 2352 | 4493 | 1412 | 1329 | 292.4 |
| | | (0.01) | (3.09) | (0.01) | (1.19) | (0.43) |
| Y33H, P67S, N106Y, | 348 | 225015 | 3259 | 204434 | 1296 | 618.8 |
| A112V | | (1.07) | (2.24) | (0.77) | (1.17) | (0.91) |
| N106Y, A112V | 349 | 6036 | 1974 | 15334 | 1108 | 409.9 |
| | | (0.03) | (1.36) | (0.06) | (1.00) | (0.61) |
| T18S, Y33H, A112V | 350 | 252647 | 1347 | 183181 | 1412 | 601.8 |
| | | (1.20) | (0.93) | (0.69) | (1.27) | (0.89) |
| P9S, Y33H, N47S, A112V | 351 | 240467 | 1418 | 203608 | 1361 | 449.1 |
| | | (1.14) | (0.98) | (0.77) | (1.22) | (0.66) |
| P42S, P67H, A112V | 352 | 204484 | 1610 | 188647 | 1174 | 530.6 |
| | | (0.97) | (1.11) | (0.71) | (1.06) | (0.78) |
| P27L, L32P, P42S, A112V | 353 | 219883 | 1963 | 84319 | 1900 | 251.6 |
| | | (1.04) | (1.35) | (0.32) | (1.71) | (0.37) |
| G98D, A112V | 354 | 4879 | 2369 | 6100 | 1729 | 387.0 |
| | | (0.02) | (1.63) | (0.02) | (1.55) | (0.57) |
| Y33H, S35P, N106Y, | 355 | 250724 | 1715 | 94373 | 1495 | 516.2 |
| A112V | | (1.19) | (1.18) | (0.36) | (1.34) | (0.76) |
| L32P, P42S, T100A, | 356 | 242675 | 1742 | 202567 | 1748 | 435.3 |
| A112V | | (1.15) | (1.20) | (0.76) | (1.57) | (0.64) |
| P27S, P45S, N106I, A112V | 357 | 223557 | 1799 | 84836 | 1574 | 277.5 |
| | | (1.06) | (1.24) | (0.32) | (1.42) | (0.41) |
| Y33H, N47K, A112V | 358 | 251339 | 1525 | 199601 | 1325 | 483.2 |
| | | (1.19) | (1.05) | (0.75) | (1.19) | (0.71) |
| Y33H, N106Y, A112V | 359 | 297169 | 1782 | 258315 | 1440 | 485.4 |
| | | (1.41) | (1.23) | (0.97) | (1.30) | (0.72) |
| K78R, D84G, A112V, | 360 | 236662 | 1638 | 24850 | 1345 | 142.5 |
| F114S | | (1.12) | (1.13) | (0.09) | (1.21) | (0.21) |
| Y33H, N47K, F54L, A112V | 361 | 14483 | 1617 | 2371 | 1353 | 352.8 |
| | | (0.07) | (1.11) | (0.01) | (1.22) | (0.52) |
| Y33H, A112V | 362 | 98954 | 1216 | 1726 | 1298 | |
| | | (0.47) | (0.84) | (0.01) | (1.17) | |
| A95V, A112V | 363 | 168521 | 2021 | 200789 | 1459 | 412.9 |
| | | (0.80) | (1.39) | (0.76) | (1.31) | (0.61) |
| R12W, A112V | 364 | 135635 | 1582 | 23378 | 1412 | 165.8 |
| | | (0.64) | (1.09) | (0.09) | (1.27) | (0.24) |
| A112V | 370 | 213576 | 1986 | 151900 | 1409 | 211.4 |
| | | (1.01) | (1.37) | (0.57) | (1.27) | (0.31) |
| Y33H, A112V | 362 | 250667 | 1628 | 230578 | 1216 | 612.7 |
| | | (1.19) | (1.12) | (0.87) | (1.09) | (0.91) |
| R12W, P27S, A112V | 365 | 3653 | 1308 | 9105 | 1051 | |
| | | (0.02) | (0.90) | (0.03) | (0.94) | |
| Y33H, V51M, A112V | 366 | 218698 | 1384 | 195450 | 1170 | 709.4 |
| | | (1.04) | (0.95) | (0.74) | (1.05) | (1.05) |
| Y33H, A112V, S118T | 367 | 219384 | 1566 | 192645 | 1313 | 396.3 |
| | | (1.04) | (1.08) | (0.73) | (1.18) | (0.59) |
| Y33H, V101A, A112V, | 368 | 5605 | 1582 | 5079 | 1197 | |
| P115S | | (0.03) | (1.09) | (0.02) | (1.08) | |
| H24R, T38N, D43G, | 369 | 227095 | 1537 | 229311 | 1336 | 858.6 |
| A112V | | (1.08) | (1.06) | (0.86) | (1.20) | (1.27) |
| A112V | 370 | 4056 | 1356 | 10365 | 986 | |
| | | (0.02) | (0.93) | (0.04) | (0.89) | |
| P27A, A112V | 371 | 193537 | 1531 | 230708 | 3084 | 355.1 |
| | | (0.92) | (1.05) | (0.87) | (2.77) | (0.52) |
| A112V, S118T | 372 | 233173 | 1659 | 121817 | 845 | 533.3 |
| | | (1.11) | (1.14) | (0.46) | (0.76) | (0.79) |
| R12W, A112V, M122I | 373 | 235935 | 1463 | 217748 | 1350 | 528.0 |
| | | (1.12) | (1.01) | (0.82) | (1.21) | (0.78) |
| Q83K, N106Y, A112V | 374 | 205948 | 2042 | 234958 | 1551 | 481.4 |
| | | (0.98) | (1.41) | (0.89) | (1.39) | (0.71) |
| R12W, P27S, A112V, | 375 | 11985 | 2667 | 12756 | 1257 | 334.4 |
| S118T | | (0.06) | (1.84) | (0.05) | (1.13) | (0.49) |
| P28S, Y33H, A112V | 376 | 4711 | 1412 | 3968 | 955 | |
| | | (0.02) | (0.97) | (0.01) | (0.86) | |
| P27S, Q90R, A112V | 377 | 3295 | 1338 | 6755 | 1048 | |
| | | (0.02) | (0.92) | (0.03) | (0.94) | |
| L15V, P27A, A112V, | 378 | 209888 | 1489 | 84224 | 1251 | 512.3 |
| S118T | | (1.00) | (1.03) | (0.32) | (1.13) | 0.76) |

### Example 11: Generation and Assessment of Engineered Cells Expressing a Transmembrane Immunomodulatory Protein

Engineered T cells were generated in which a transmembrane immunomodulatory protein (TIP) containing an extracellular domain (ECD) containing either a variant CD80 or ICOSL affinity-modified IgSF domain as described above was co-expressed with a chimeric antigen receptor (CAR). The TIP also contained a transmembrane domain and a cytoplasmic domain of the corresponding wild-type CD80 or ICOSL transmembrane protein sequence. The immunomodulatory activity of the engineered cells was compared to cells that only expressed the CAR or cells that co-expressed the corresponding wild-type CD80 or ICOSL transmembrane protein with the CAR.

The exemplary CD80-TIP was a variant CD80 having an affinity-modified IgSF domain containing amino acid mutations in the IgV and IgC domains corresponding to I67T/L70Q/A91G/T120S with reference to positions in the CD80 extracellular domain set forth in SEQ ID NO:28 and a transmembrane and cytoplasmic domain corresponding to residues 243-288 of SEQ ID NO:1. The amino acid sequence of the exemplary CD80-TIP is set forth in SEQ ID NO:381 and is encoded by the sequence of nucleotides set forth in SEQ ID NO:382. The corresponding wild-type CD80 transmembrane protein had the sequence of amino acids set forth as amino acid residues 35-288 of SEQ ID NO:1 and encoded by the sequence of amino acids set forth in SEQ ID NO: 391.

The exemplary ICOSL-TIP was a variant ICOSL having an affinity-modified IgSF domain containing amino acid mutations in the IgV domain corresponding to N52H/I143T with reference to positions in the ICOSL extracellular domain set forth in SEQ ID NO:32 and a transmembrane and cytoplasmic domain corresponding to residues 257-302 of SEQ ID NO:5. The amino acid sequence of the exemplary ICOSL-TIP is set forth in SEQ ID NO:383 and is encoded by the sequence of nucleotides set forth in SEQ ID NO:384. The corresponding wild-type ICOSL transmembrane protein had the sequence of amino acids set forth as amino acid residues 19-302 of SEQ ID NO:5 and encoded by the sequence of amino acids set forth in SEQ ID NO: 392.

The TIP containing the affinity-modified domain or the wild-type transmembrane protein containing a corresponding non-affinity modified IgSF domain were co-expressed in T cells with a 1^{st} generation chimeric antigen receptor (CAR) containing a CD3zeta intracellular signaling domain. The 1^{st} generation CAR included an ScFv specific for CD19 (SEQ ID NO:385), a hinge and transmembrane domain derived from CD8 (SEQ ID NO:386) and an intracellular signaling domain derived from CD3zeta (set forth in SEQ ID NO:387). The nucleotide sequence encoding the CD19 scFv—CD3zeta CAR is set forth in SEQ ID NO:388.

Nucleic acid molecules encoding the CAR alone or also encoding one of the exemplary TIPs or wild-type transmembrane proteins separated from the CAR by a self-cleaving T2A sequence (SEQ ID NO:390 and encoded by the sequence of nucleotides set forth in SEQ ID NO:389) were generated. Exemplary constructs contained nucleic acid sequences set forth in Table 19. As a control, a nucleic acid construct encoding a 2^{nd} generation CAR additionally containing a CD28 costimulatory domain also was generated (CD19 scFv—CD28—CD3zeta).

TABLE 19
Nucleic Acid Constructs

| | CAR | T2A Linker | TIP |
|---|---|---|---|
| | (SEQ ID | (SEQ ID | (SEQ ID |
| | NO) | NO) | NO) |
| CD19 scFv - CD3zeta | + | — | — |
| | (386) | | |
| CD19 scFv - CD3zeta - | + | + | Wildtype CD80 |
| T2A - B7-1 | (386) | (389) | (391) |
| CD19 scFv - CD3zeta - | + | + | CD80 TIP |
| T2A - B7-1_TIP | (386) | (389) | (382) |
| CD19 scFv - CD3zeta - | + | + | Wildtype |
| T2A - ICOSL | (386) | (389) | ICOSL |
| | | | (392) |
| CD19 scFv - CD3zeta - | + | + | ICOSL TIP |
| T2A - ICOSL_TIP | (386) | (389) | (384) |

The nucleic acid molecules were individually cloned into a lentiviral vector, which was used to transduce T cells isolated from human PBMC samples obtained from three different healthy donors. Lentivirus particles containing the nucleic acid sequences were produced after co-transfection of HEK293 cells with the vectors and lentivirus packaging constructs. The lentivirus particles were collected from the culture medium by ultracentrifugation and titered by qRT-PCR. Human peripheral blood mononuclear cells (PBMC) were isolated from three normal blood donors using density sedimentation. The PBMC were cultured overnight with anti-CD3 and anti-CD28 antibodies and IL-2, then transduced with the lentivirus preparations at a multiplicity of infection of 5:1. The lentiviral vectors encoding the control 2^{nd} generation CAR was only used to transduce cells from one donor.

After two weeks (14 days) of culture, the cells were analyzed for cytotoxicity following co-culture with target antigen-expressing cells using the Acea Real-Time Cell Analyzer (RTCA), which measures the impedance variations in the culture media of a 96-well microelectronic plate (E-plate), and shows the changes in cell number and morphology in a real-time plot. CD19-expressing HeLa target cells (HeLa-CD19) were seeded into a 96-well E-plate and the impedance of each monolayer was monitored for 24 hours using the RTCA system. The engineered T cells were added to the wells at an effector:target ratio of 10:1 and the wells were monitored for another 48 hours. The results were displayed and recorded as Cell Index (CI) value derived from the change in measured electrical impedance and were then ratio transformed by dividing the CI readouts of all wells at all time points over the CI value of individual wells at a same time (base-time) to obtain a normalized cell index value representing the percentage of the value at the base-time (see Zhang et al. “Introduction to the Data Analysis of the Roche xCELLigence® System with RTCA Package.” Bioconductor. May 3, 2016, bioconductor.org/packages/devel/bioc/vignettes/RTCA/inst/doc/aboutRTCA.pdf. Accessed Sep. 9, 2016). In this assay, a decrease in the impedance of a monolayer reflects killing of the target cells by the transduced cells.

The results showed that decreased impedance was observed in cells expressing the 1^{st} generation CAR compared to non-transduced T cells, although the degree of decreased impedance for cells expressing the 1^{st} generation CAR was less than cells expressing the 2^{nd} generation CAR. The decreased impedance in cells expressing the 1^{st} generation CAR continued generally for up to the first 8 hours of the assay, while only the 2^{nd} generation CAR-expressing cells continued to decrease the impedance thereafter.

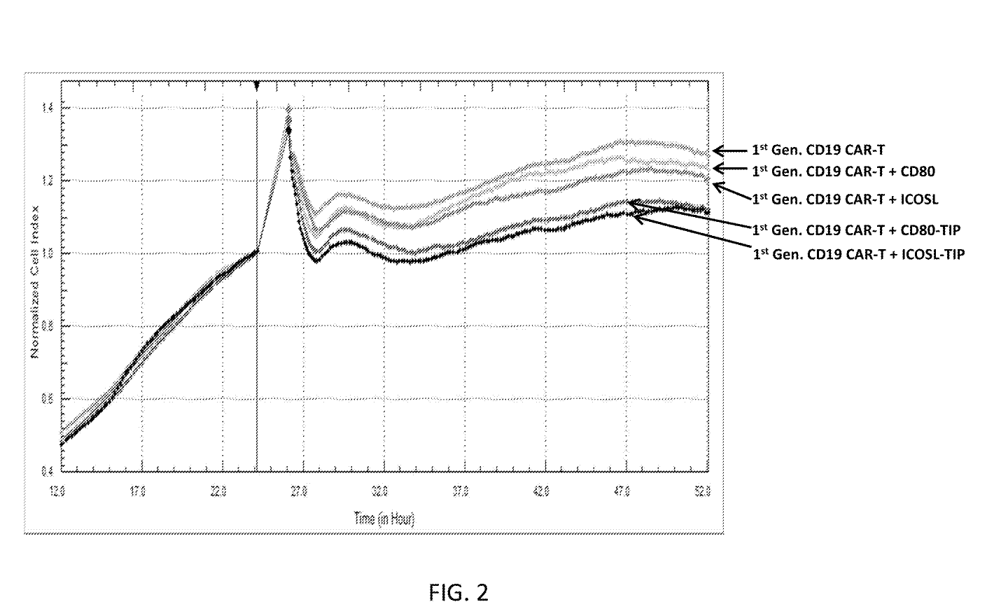

As shown in FIG. 2, in one donor, each of the cells co-expressing the TIP or corresponding wild-type transmembrane protein with the 1^{st} generation CAR exhibited a greater decrease in impedance, indicating greater cytotoxic activity, compared to cells only expressing the 1^{st} generation CAR. Further, the results showed that the cytotoxic activity was greater in CAR-expressing cells that co-expressed the CD80-TIP or ICOSL-TIP relative to CAR-expressing cells that co-expressed the corresponding wild-type CD80 or ICOSL transmembrane proteins containing a non-affinity modified IgSF domain. The observed results of these TIP-engineered cells showed that cytotoxic activity in cells co-expressing the CD80-TIP or ICOSL-TIP with the CAR exhibit increased activity to modulate the cytotoxic immune response of antigen-specific T cells, such as the CAR-expressing T cells.

In the other two donors, the cells expressing the CD80-TIP did not result in a greater decreased impedance compared to cells expressing the corresponding wild-type CD80 transmembrane protein. In one donor, there were not enough cells to transduce with the wild-type transmembrane protein construct, although in this donor the ICOS-L TIP gave the best cytotoxicity compared to the other constructs tested. In the other donor, the cells expressing the ICOS-L-TIP did not result in a greater decreased impedance compared to cells expressing the corresponding wild-type ICOS-L transmembrane protein. In the tested cells, all cells co-expressing either a CD80-TIP, ICOSL-TIP or corresponding wild type transmembrane protein with the CAR exhibited greater cytotoxic activity than cells only expressing the 1st generation CAR. The differences in the results observed among donors may be related to the differences in the T cells among the donors, differences in expression levels of the various engineered proteins on the surface of the cells, the particular conditions used in this exemplary assay for assessing killing in cells (e.g. assessing Day 14 transduced cells, assessing a single effector:target cell ratio) or other factors.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A transmembrane immunomodulatory protein (TIP) comprising:
(i) an ectodomain comprising at least one non-immunoglobulin affinity-modified immunoglobulin superfamily (IgSF) domain comprising one or more amino acid substitution(s) in a wild-type IgSF domain, wherein the at least one affinity-modified IgSF domain specifically binds at least one cell surface cognate binding partner of the wild-type IgSF domain; and
(ii) a transmembrane domain.

2. The transmembrane immunomodulatory protein of claim 1, wherein the at least one cell surface cognate binding partner is expressed on a mammalian cell.

3. The transmembrane immunomodulatory protein of claim 2, wherein the mammalian cell is an antigen presenting cell (APC), a tumor cell, or a lymphocyte, which optionally is a T-cell.

4. The transmembrane immunomodulatory protein of any of claims 1-3, wherein the mammalian cell is a mouse, rat, cynomolgus monkey, or human cell.

5. The transmembrane immunomodulatory protein of any of claims 1-4, wherein the at least one affinity modified IgSF domain has increased binding affinity to the at least one cell surface cognate binding partner compared with the reference wild-type IgSF domain.

6. The transmembrane immunomodulatory protein of any of claims 2-5, wherein specific binding of the transmembrane immunomodulatory protein comprising the at least one affinity-modified IgSF domain modulates immunological activity of the mammalian cell compared with the reference transmembrane domain comprising the wild-type IgSF domain.

7. The transmembrane immunomodulatory protein of any of claims 2-6, wherein specific binding of the transmembrane immunomodulatory protein comprising the at least one affinity-modified IgSF domain increases immunological activity of the mammalian cell compared with the reference transmembrane domain comprising the wild-type IgSF domain.

8. The transmembrane immunomodulatory protein of any of claims 2-6, wherein specific binding of the transmembrane immunomodulatory protein attenuates immunological activity of the mammalian cell compared with the reference transmembrane domain comprising the wild-type IgSF domain.

9. The transmembrane protein of any of claims 1-8, wherein the wild-type IgSF domain is from an IgSF family member of a family selected from Signal-Regulatory Protein (SIRP) Family, Triggering Receptor Expressed On Myeloid Cells Like (TREML) Family, Carcinoembryonic Antigen-related Cell Adhesion Molecule (CEACAM) Family, Sialic Acid Binding Ig-Like Lectin (SIGLEC) Family, Butyrophilin Family, B7 family, CD28 family, V-set and Immunoglobulin Domain Containing (VSIG) family, V-set transmembrane Domain (VSTM) family, Major Histocompatibility Complex (MHC) family, Signaling lymphocytic activation molecule (SLAM) family, Leukocyte immunoglobulin-like receptor (LIR), Nectin (Nec) family, Nectin-like (NECL) family, Poliovirus receptor related (PVR) family, Natural cytotoxicity triggering receptor (NCR) family, T cell immunoglobulin and mucin (TIM) family or Killer-cell immunoglobulin-like receptors (KIR) family.

10. The transmembrane immunomodulatory protein of any of claims 1-9, wherein the wild-type IgSF domain is from an IgSF member selected from CD80, CD86, PD-L1, PD-L2, ICOS Ligand, B7-H3, B7-H4, CD28, CTLA4, PD-1, ICOS, BTLA, CD4, CD8-alpha, CD8-beta, LAG3, TIM-3, CEACAM1, TIGIT, PVR, PVRL2, CD226, CD2, CD160, CD200, CD200R or Nkp30.

11. The transmembrane immunomodulatory protein of any of claims 1-10, wherein the wild-type IgSF domain is a human IgSF member.

12. The transmembrane immunomodulatory protein of any of claims 1-11, wherein the at least one affinity modified IgSF domain has at least 90% sequence identity to a wild-type IgSF domain or a specific binding fragment thereof contained in the sequence of amino acids set forth in any of SEQ ID NOS: 1-27.

13. The transmembrane immunomodulatory protein of any of claims 1-12, wherein the transmembrane immunomodulatory protein has at least 90% sequence identity to the amino acid sequence selected from any of SEQ ID NOS: 393-419.

14. The transmembrane immunomodulatory protein of any of claims 1-13, wherein the at least one cell surface cognate binding partner is a stimulatory receptor expressed on a T-cell and the at least one affinity-modified IgSF domain has increased binding affinity to the stimulatory receptor compared to the affinity of the wild-type IgSF domain.

15. The transmembrane immunomodulatory protein of claim 14, wherein binding of the affinity-modified IgSF domain to the stimulatory receptor increases immunological activity of the T-cell.

16. The transmembrane immunomodulatory protein of claim 14 or claim 15, wherein the stimulatory receptor is CD28, ICOS or CD226.

17. The transmembrane immunomodulatory protein of any one of claims 14-16, wherein the at least one affinity-modified IgSF domain is an affinity modified B7-1 IgSF domain and the stimulatory receptor is CD28.

18. The transmembrane immunomodulatory protein of any one of claims 14-16, wherein the at least one affinity-modified IgSF domain is an affinity modified ICOSL IgSF domain and the stimulatory receptor is ICOS.

19. The transmembrane immunomodulatory protein of any one of claims 14-16, wherein the affinity-modified IgSF domain is an affinity modified ICOSL IgSF domain and the stimulatory receptor is CD28.

20. The transmembrane immunomodulatory protein of any one of claims 14-16, 18 and 19, wherein the at least one affinity-modified IgSF domain is an affinity-modified ICOSL IgSF domain that has increased binding affinity to at least one of: ICOS and CD28.

21. The transmembrane immunomodulatory protein of any one of claims 14-16- and 18-20, wherein the affinity modified IgSF domain is an affinity modified ICOSL IgV IgSF domain with increased binding affinity to both ICOS and CD28.

22. The transmembrane immunomodulatory protein of any one of claims 17-21, wherein the affinity-modified IgSF domain does not substantially specifically bind to CTLA-4 or exhibits decreased binding affinity to CTLA-4 compared to the wild-type IgSF domain.

23. The transmembrane immunomodulatory protein of any of claims 1-22, wherein the at least one affinity-modified IgSF domain specifically binds to no more than one cell surface cognate binding partner.

24. The transmembrane immunomodulatory protein of any of claims 1-23, wherein the transmembrane immunomodulatory protein specifically binds to no more than one cell surface cognate binding partner.

25. The transmembrane immunomodulatory protein of any of claims 1-22, wherein the at least one affinity-modified domain specifically binds to at least two cell surface cognate binding partners.

26. The transmembrane immunomodulatory protein of claim 25, wherein:
the first cell surface cognate binding partner is a stimulatory receptor expressed on a T cell; and
the second cell surface cognate binding partner is an inhibitory ligand of an inhibitory receptor, wherein the inhibitory receptor is expressed on a T-cell.

27. The transmembrane immunomodulatory protein of claim 26, wherein binding of the affinity-modified domain to the inhibitory ligand competitively inhibits binding of the inhibitory ligand to the inhibitory receptor.

28. The transmembrane immunomodulatory protein of claim 26 or claim 27, wherein:
the inhibitory receptor is PD-1, CTLA-4, LAG-3, TIGIT, CD96, CD112R, BTLA, CD160 or TIM-3; or
the ligand of the inhibitory receptor is PD-L1, PD-L2, B7-1, B7-2, HVEM, MHC class II, PVR, CEACAM-1 or GAL9.

29. The transmembrane immunomodulatory protein of any one of claims 26-28, wherein the affinity modified IgSF domain is an affinity modified B7-1 domain and the stimulatory receptor is CD28.

30. The transmembrane immunomodulatory protein of claim 29, wherein the inhibitory ligand is PD-L1 and the inhibitory receptor is PD-1.

31. The transmembrane immunomodulatory protein of claim 29 or claim 30, wherein the affinity-modified IgSF domain exhibits decreased binding affinity to CTLA-4 compared to the wild-type IgSF domain for CTLA-4.

32. The transmembrane immunomodulatory protein of any one of claims 29-31, wherein the affinity-modified IgSF domain does not substantially specifically bind to CTLA-4.

33. The transmembrane immunomodulatory protein of any of claims 1-13, wherein the affinity modified IgSF domain is an affinity modified CD155 IgSF domain or an affinity modified CD112 IgSF domain and the stimulatory receptor is CD226.

34. The transmembrane immunomodulatory protein of claim 33, wherein the affinity-modified IgSF domain exhibits decreased binding affinity to TIGIT (T-cell immunoreceptor with Ig and ITIM domains) compared to the affinity of the wild-type IgSF domain.

35. The transmembrane immunomodulatory protein of any of claims 1-13, wherein the at least one affinity-modified IgSF domain specifically binds to a cell surface cognate binding partner that is a tumor specific antigen.

36. The transmembrane immunomodulatory protein of claim 35, wherein the tumor specific antigen is B7-H6.

37. The transmembrane immunomodulatory protein of claim 35 or claim 36, wherein the affinity-modified IgSF domain is an affinity modified Nkp30 IgSF domain.

38. The transmembrane immunomodulatory protein of any one of claims 1-37, wherein the at least one affinity-modified IgSF domain is a first affinity-modified IgSF domain and the ectodomain comprises a second affinity-modified IgSF domain.

39. The transmembrane immunomodulatory protein of claim 38, wherein the first and second affinity-modified IgSF domain are different.

40. The transmembrane immunomodulatory protein of claim 38 or claim 39, wherein the first affinity-modified IgSF domain and the second affinity-modified IgSF domain each comprise one or more amino acid different substitutions in the same wild-type IgSF domain.

41. The transmembrane immunomodulatory protein of claim 38 or claim 39, wherein the first affinity-modified IgSF domain and the second affinity-modified IgSF domain each comprise one or more amino acid substitutions in a different wild-type IgSF domain.

42. The transmembrane immunomodulatory protein of any of claims 1-41, wherein the transmembrane immunomodulatory protein further comprises an endodomain or cytoplasmic signaling domains.

43. The transmembrane immunomodulatory protein of claim 42, wherein the endodomain is the endodomain from the wild-type IgSF member comprising the wild-type IgSF domain or is a functionally active portion thereof.

44. The transmembrane immunomodulatory protein of claim 42, wherein the transmembrane immunomodulatory protein is a chimeric receptor, wherein the endodomain is not the endodomain from the wild-type IgSF member comprising the wild-type IgSF domain.

45. The transmembrane immunomodulatory protein of claim 42 or claim 44, wherein the endodomain comprises at least one ITAM (immunoreceptor tyrosine-based activation motif)-containing signaling domain.

46. The transmembrane immunomodulatory protein of any of claims 42, 44 and 45, wherein the endodomain comprises a CD3-zeta signaling domain.

47. The transmembrane immunomodulatory protein of claim 45 or claim 46, wherein the endodomain further comprises at least one of: a CD28 costimulatory domain, an ICOS signaling domain, an OX40 signaling domain, and a 41BB signaling domain.

48. The transmembrane immunomodulatory protein of any of claims 1-13, wherein the wild-type IgSF domain is from an IgSF member that is an inhibitory receptor comprising an ITIM signaling domain.

49. The transmembrane immunomodulatory protein of claim 48, wherein the inhibitory receptor is PD-1, CTLA-4, LAG3, TIGIT, TIM-3, or BTLA and the at least one affinity-modified IgSF domain is an affinity-modified IgSF domain of PD-1, CTLA-4, LAG3, TIGIT, TIM-3, or BTLA, respectively.

50. The transmembrane immunomodulatory protein of claim 48 or claim 49, wherein the inhibitory receptor is PD-1 and the at least one affinity-modified IgSF domain is an affinity-modified IgSF of PD-1.

51. The transmembrane immunomodulatory protein of any of claims 48-50, wherein the affinity-modified IgSF domain has increased binding affinity for a trans surface cognate binding partner compared to the wildtype IgSF domain, whereby the increased binding affinity competitively inhibits binding of the trans surface cognate binding partner to the inhibitory receptor.

52. The transmembrane immunomodulatory protein of any of claims 48-51, wherein the transmembrane immunomodulatory protein does not comprise an endodomain, ITIM or cytoplasmic signaling domains.

53. The transmembrane immunomodulatory protein of any of claims 1-52, wherein the affinity modified IgSF domain differs by no more than ten amino acid substitutions from the wildtype IgSF domain.

54. The transmembrane immunomodulatory protein of any of claims 1-53, wherein the affinity modified IgSF domain differs by no more than five amino acid substitutions from the wildtype IgSF domain.

55. The transmembrane immunomodulatory protein of any of claims 1-54, wherein the affinity-modified IgSF domain is or comprises an affinity modified IgV domain, affinity modified IgC1 domain or an affinity modified IgC2 domain or is a specific binding fragment thereof comprising the one or more amino acid substitutions.

56. The transmembrane immunomodulatory protein of any of claims 1-55, wherein the ectodomain further comprises one or more non-affinity modified IgSF domains.

57. The transmembrane immunomodulatory protein of claim 56, wherein the one or more non-affinity modified IgSF domains is from a wild-type IgSF member comprising the wild-type IgSF domain.

58. The transmembrane immunomodulatory protein of any of claims 1-57, wherein the transmembrane domain is the native transmembrane domain from the corresponding wild-type IgSF member.

59. The transmembrane immunomodulatory protein of any of claims 1-57, wherein the transmembrane domain is not the native transmembrane domain from the corresponding wild-type IgSF member.

60. The transmembrane immunomodulatory protein of claim 59, wherein the transmembrane protein is a transmembrane protein derived from CD8.

61. A recombinant nucleic acid encoding a transmembrane immunomodulatory proteins of any of claims 1-60.

62. A recombinant expression vector comprising the nucleic acid of claim 61.

63. A recombinant host cell comprising the expression vector of claim 62.

64. A recombinant host cell comprising the nucleic acid of claim 61.

65. The recombinant host cell of claim 63 or claim 64, wherein the host cell is a mammalian host cell.

66. The recombinant host cell of any of claims 63-65, wherein the mammalian host cell is a human host cell.

67. An engineered cell comprising the transmembrane immunomodulatory protein of any of claims 1-60.

68. The engineered cell of claim 67, wherein the cell is an immune cell.

69. The engineered cell of claim 67 or claim 68, wherein the cell is a lymphocyte.

70. The engineered cell of claim 69, wherein the lymphocyte is a T cell, a B cell or an NK cell.

71. The engineered cell of any of claims 67-70, wherein the cell is a T cell.

72. The engineered cell of claim 71, wherein the T cells is CD4+ or CD8+.

73. The engineered cell of claim 67 or claim 68, wherein the cell is an antigen presenting cell.

74. The engineered cell of any of claims 67-73, further comprising a chimeric antigen receptor (CAR) or an engineered T-cell receptor (TCR).

75. A pharmaceutical composition comprising the cell of any of claims 67-74 and a pharmaceutically acceptable carrier.

76. The pharmaceutical composition of claim 75 that is sterile.

77. A method of modulating an immune response in a mammalian subject, comprising administering a cell of any of claims 67-74 or a pharmaceutical composition of claim 75 or claim 76 to the subject.

78. The method of claim 76 or claim 77, wherein modulating the immune response treats a disease or disorder in the subject.

79. The method of any of claims 77-78, wherein the modulated immune response is increased.

80. The method of claim 78 or claim 79, wherein the disease or disorder is a tumor.

81. The method of any of claims 78-80, wherein the disease or disorder is a cancer.

82. The method of any of claims 78-81, wherein the disease or disorder is melanoma, lung cancer, bladder cancer, or a hematological malignancy.

83. The method of any of claims 77-78, wherein the modulated immune response is decreased.

84. The method of claim 78 or claim 83, wherein the disease or disorder is an inflammatory disease or condition.

85. The method of any of claims 78, 83 and 84, wherein the disease or condition is Crohn's disease, ulcerative colitis, multiple sclerosis, asthma, rheumatoid arthritis, or psoriasis.

86. The method of any of claims 77-85, wherein the subject is human.

87. The method of any of claims 77-86, wherein the cell is autologous to the subject.

88. The method of any of claims 77-87, wherein the cell is allogenic to the subject.
